(19) United States
(12) Patent Application Publication
(10) Pub. No.: US 2003/0133936 A1
(43) Pub. Date: Jul. 17, 2003

(54) CD25markers and uses thereof

(76) Inventors: Michael Chapman Byrne, Brookline, MA (US); Ethan Menahem Shevach, Rockville, MD (US); Mary Collins, Natick, MA (US); Rebecca Suzanne McHugh, Silver Spring, MD (US); Matthew James Whitters, Hudson, MA (US); Deborah Ann Young, Melrose, MA (US)

Correspondence Address: FITZPATRICK CELLA HARPER & SCINTO, 30 ROCKEFELLER PLAZA, NEW YORK, NY, 10112, US

(21) Appl. No.: 10/194,754

(22) Filed: Jul. 12, 2002

Related U.S. Application Data
(60) Provisional application No. 60/304,827, filed on Jul. 12, 2001.

(51) Int. Cl. A61K 39/395; G01N 33/567; A61K 31/00

(52) U.S. Cl. 424/146.1; 514/1; 435/7.21

(57) ABSTRACT

The present invention is directed to methods and compositions for the identification of novel targets for diagnosis, prognosis, therapeutic intervention and prevention of autoimmune disorders, transplant rejection and cancer. In particular, the present invention is directed to the identification of novel targets which are CD25⁺ differential markers. The present invention is further directed to methods of high-throughput screening for test compounds capable of modulating the activity of proteins encoded by the novel targets. Moreover, the present invention is also directed to methods that can be used to assess the efficacy of test compounds and therapies for the ability to inhibit an autoimmune disorder or transplant rejection. Methods for determining the long term prognosis in a subject are also provided.

This invention claims the benefit of U.S. Provisional Patent Application No. 60/304,827, filed Jul. 12, 2001.

This invention was made with Government support under NIH Intramural Research Project #Z01-AI-00224. The Government has certain rights in the invention.

### BACKGROUND OF THE INVENTION

### Field of the Invention

The present invention is directed to novel methods for diagnosis, treatment and prognosis of autoimmune disorders using CD25⁺ differentially expressed genes. The present invention is further directed to novel therapeutics and therapeutic targets and to methods of screening and assessing test compounds for the intervention and prevention of autoimmune disorders as well as transplant immunosuppression methods. The invention is also directed to novel cancer treatments related to CD25⁺ differential markers.

Generally, T lymphocytes are responsible for cell-mediated immunity and play a regulatory role by enhancing or suppressing the responses of other white blood cells.

T lymphocytes have also long been thought to play a role in suppression of the immune response. See e.g., Gershon et al., Immunology (1970) 18:723-35. However, the target antigens for these suppressor or regulatory cells are still poorly defined.

One population of regulatory T cells, which is generated in the thymus is distinguishable from effector T cells by the expression of unique membrane antigens. These regulatory T cells make up a sub-population of CD4⁺ T cells which co-express the CD25 (also known as the IL-2R α-chain) antigen. Cotransfer of or reconstitution by CD25⁺ T cells is associated with prevention of inflammatory lesions and autoimmunity. See E. M. Shevach, Regulatory T cells in Autoimmunity, Ann. Rev. Immunol. (2000) 18:423-449, and references therein. CD4⁺CD25⁺ T cells have also been associated with inhibition of T cell activation in vitro, and adoptive suppression of CD4⁺CD25⁻ T cells in coculture. Id.

More than two decades ago it was demonstrated that some self-reactive T cells escape mechanisms of central tolerance and exist in the periphery under the control of thymic-derived regulatory T cells. Sakaguchi and colleagues, in 1995, demonstrated that a small population of CD4⁺T cells that naturally express the alpha chain of the IL-2R (CD25) relates to the control of organ-specific autoreactive T cells. See Sakaguchi, S., Sakaguchi, N., Asano, M., Itoh, M., and Toda, M. (1995). J. Immunology 155, 1151-1164. Since then, many attempts have been made to define the activation of and suppression by these CD4⁺CD25⁺ T cells. Several in vitro studies have revealed that these cells suppress proliferation of CD4⁺T cells to both mitogens and antigen by turning off transcription of IL-2. See Takahashi, T. et al International Immunology 10, 1969-1980 (1998); Thornton, A. M., and Shevach, E. M.., J. Exp. Med. 188, 287-296 (1998). In vivo, co-transfer of CD4⁺CD25⁺ cells with autoreactive CD4⁺T cells is sufficient to suppress both the induction and effector phase of organ-specific autoimmunity. Suri-Payer, E. et al, European J. Immunology 29, 669-677 (1999); Suri-Payer, E. et al,. J. Immunology 160, 1212-1218 (1998). Other properties of the CD4⁺CD25⁺ T cells include hypo-responsiveness to T Cell Receptor (TCR) stimulation in the absence of exogenous IL-2, suppression via cell-cell interaction, and the requirement for TCR signaling to exert their suppression, but once they have been activated, their suppressive function is independent of antigenic stimulus. It has also been demonstrated that the mere acquisition of CD25 expression, as can be achieved by stimulation of CD4⁺CD25⁻ T cells, does not induce the suppressive phenotype. Further, these cells are known to exist in humans. See Shevach E. M, J. Exp. Med.193:F1-F6 (2001).

Recently, one study demonstrated that altered thymic selection was required for generation of regulatory CD4⁺CD25⁺ T cells. Jordan, M. S.,et al, Nature Immunology 2, 301-306 (2001). In addition, studies with various knockout mice demonstrate that molecules involved in IL-2 synthesis and responsiveness are required for generation of these cells. IL-2−/−, IL-2Rb−/−, B7-1/2 double−/− and CD28⁻/− all have severe reduction in CD4⁺CD25⁺ cells, with resulting lymphadenopathy and hyperproliferation in the periphery of some of these mice. See Papiernik, M., et al, Intl. Immunology 10, 371-378 (1998); Salomon, B. et al, Immunity 12, 431-440 (2000); Kumanogoh, A. et al. J. Immunology 166, 353-360 (2001).

Despite all of these efforts, however, the art has failed to determine the antigen specificity, molecules involved in acquisition of suppression, and the cell surface molecules or short acting cytokines involved in the effector phase of suppression. Further, the molecular targets of CD25⁺ T cells in modulating autoimmunity remain largely unknown. Accordingly, there is a need in the art for molecular targets involved in CD25⁺ T cell suppression.

The present invention fills this void by providing CD25⁺ differential markers that serve as targets for therapeutic intervention for autoimmune disorders and transplant rejection as well as markers for diagnostic and prognostic methods. The invention also provides compositions and methods for screening test compounds useful for treating, diagnosing or preventing autoimmune disorders, transplant rejection. The invention further provides novel cancer treatment and screening methods related to CD25⁺ differential markers.

### SUMMARY OF THE INVENTION

In one embodiment, the invention provides a method of assessing the efficacy of a test compound for inhibiting an autoimmune disorder or transplant rejection in a subject, the method comprising the step of comparing: a) expression of Glucocorticoid Induced TNF Receptor (“GITR”) in a first sample obtained from the subject, wherein the first sample is exposed to the test compound, and b) expression of GITR in a second sample obtained from the subject, wherein the second sample is not exposed to the test compound, wherein a substantially modulated level of expression GITR in the first sample, relative to the second sample, is an indication that the test compound is efficacious for inhibiting the autoimmune disorder or transplant rejection in the subject.

In another embodiment, the invention provides a method of assessing the efficacy of a therapy for inhibiting an autoimmune disorder or transplant rejection in a subject, the method comprising the steps of comparing: a) expression of GITR in a first sample obtained from the subject prior to providing at least a portion of the therapy to the subject, and b) expression of GITR in a second sample following provision of the portion of the therapy, wherein a substantially modulated level of expression of GITR in the second sample, relative to the first sample, is an indication that the therapy is efficacious for inhibiting the autoimmune disorder or transplant rejection in the subject.

In yet another embodiment, the invention provides a method of high-throughput screening for test compounds capable of modulating the activity of a GITR protein, the method comprising: a) contacting the GITR protein with a plurality of test compounds; b) detecting binding of one of the test compounds to the GITR protein, relative to other test compounds; and c) correlating the amount of binding of the test compound to the GITR protein with the ability of the test compound to modulate the activity of the GITR protein, wherein binding indicates that the test compound is capable of modulating the activity of the GITR protein.

In yet another embodiment, the invention provides a method of high-throughput screening for test compounds capable of inhibiting an autoimmune disorder or transplant rejection in a subject, the method comprising: a) combining a GITR protein, a specific factor which binds to a GITR protein, and a test compound; b) selecting one of the test compounds which modulates the binding of GITR and the specific factor as compared to other test compounds; and c) correlating the amount of modulation of binding with the ability of the test compound to inhibit the autoimmune disorder or transplant rejection, wherein modulation of binding of GITR protein and the specific factor indicates that the test compound is capable of inhibiting the autoimmune disorder or transplant rejection.

In yet another embodiment, the invention provides a method of screening for a test compound capable of interfering with the binding of a GITR protein and a specific factor which binds to the protein, the method comprising a) combining the GITR protein, a test compound and the specific factor which binds to the GITR protein; and b) determining the binding of the GITR protein and the specific factor; and c) correlating the amount of binding with the ability of the test compound to interfere with binding, wherein a decrease in binding of the GITR protein and the specific factor in the presence of the test compound as compared to the absence of the test compound indicates that the test compound is capable of inhibiting binding.

In another embodiment, the invention provides a method of screening test compounds for inhibitors of an autoimmune disorder or transplant rejection in a subject, the method comprising the steps of: a) obtaining a sample comprising cells; b) contacting an aliquots of the sample with one of a plurality of test compounds; c) comparing the expression levels GITR in each of the aliquots; and d) selecting one of the test compounds which substantially modulates the level of expression of a GITR expression in the aliquot containing that test compound, relative to other test compounds.

In another embodiment, the invention provides a method of determining the severity of an autoimmune disorder or transplant rejection in a subject, the method comprising the step of comparing: a) a level of expression of GITR in a sample from the subject; and b) a normal level of expression of GITR in a control sample, wherein an abnormal level of expression of GITR in the sample from the subject relative to the normal levels is an indication that the subject is suffering from a severe autoimmune disorder or transplant rejection.

In another embodiment, the invention provides a method of treating a subject diagnosed with an autoimmune disorder or transplant rejection in a subject, the method comprising administering a composition comprising a GITR polypeptide and a pharmaceutically acceptable carrier.

In another embodiment, the invention provides a method of treating a subject diagnosed with an autoimmune disorder or transplant rejection in a subject, the method comprising administering a composition comprising a GITR polynucleotide, a delivery vehicle and a pharmaceutically acceptable carrier.

In yet another embodiment, the invention provides a method of modulating a level of expression of GITR, the method comprising providing to cells of a subject an antisense oligonucleotide complementary to a GITR polynucleotide.

In another embodiment, the invention provides a method of modulating a level of expression of GITR, the method comprising providing to cells of a subject a protein corresponding to GITR.

In another embodiment, the invention provides a method of modulating activity of GITR the method comprising providing to cells of a subject an antibody which specifically binds to the GITR protein.

In yet another embodiment, the invention provides a method of localizing a therapeutic moiety to tissue having an autoimmune disorder or transplant rejection comprising: 1) linking the therapeutic moiety to a GITR binding partner selected from the group consisting of an antibody which is specific to a GITR protein and a GITR ligand; and 2) administering, to a subject in need of treatment, the therapeutic moiety linked to the binding partner.

In another embodiment, the invention provides a biochip comprising a GITR marker and at least 5 or more CD25⁺ differential markers (listed in Table I, or a homolog thereof), or homologs thereof, wherein the biochip is utilized in high-throughput screening assays for inhibition an autoimmune disorder or transplant rejection.

In another embodiment, the invention provides a composition capable of inhibiting an autoimmune disorder or transplant rejection in a subject, the composition comprising a GITR polypeptide and a pharmaceutically acceptable carrier.

In another embodiment, the invention provides a composition capable of inhibiting an autoimmune disorder or transplant rejection in a subject, the composition comprising a GITR polynucleotide, a delivery vehicle and a pharmaceutically acceptable carrier.

In another embodiment, the invention provides a therapeutic target for the inhibition of an autoimmune disorder or transplant rejection, wherein the therapeutic target comprises a GITR marker gene.

In another embodiment, the invention provides a therapeutic target for the inhibition of an autoimmune disorder or transplant rejection, wherein the therapeutic target comprises a protein encoded by a GITR marker gene.

In another embodiment, the invention provides a kit for determining the long term prognosis in a subject having an autoimmune disorder or transplant rejection, the kit comprising a polynucleotide probe wherein the probe specifically binds to a transcribed GITR polynucleotide.

In yet another embodiment, the invention provides a kit for assessing the suitability of each of a plurality of compounds for inhibiting an autoimmune disorder or transplant rejection in a subject, the kit comprising: a) the plurality of compounds; and b) a reagent for assessing expression of GITR

In another embodiment, the invention provides a kit for determining the long term prognosis in a subject having an autoimmune disorder or transplant rejection, wherein the kit comprises an antibody which specifically binds with a GITR protein.

In another embodiment, the invention provides a kit comprising a biochip and a computer readable medium, wherein the biochip comprises a GITR marker and at least 5 CD⁺differential markers (listed in Table I, or a homolog thereof) and wherein the computer readable medium contains the same CD25⁺ differential markers in computer readable form.

In another embodiment, the invention provides a method of assessing the efficacy of a test compound for inhibiting a cancer or proliferative disorder in a subject, the method comprising the step of comparing: a) expression of GITR in a first sample obtained from the subject, wherein the first sample is exposed to the test compound, and b) expression of GITR in a second sample obtained from the subject, wherein the second sample is not exposed to the test compound, wherein a substantially modulated level of expression GITR in the first sample, relative to the second sample, is an indication that the test compound is efficacious for inhibiting the cancer in the subject.

In another embodiment, the invention provides a method of assessing the efficacy of a therapy for inhibiting a cancer or proliferative disorder in a subject, the method comprising the steps of comparing: a) expression of GITR in a first sample obtained from the subject prior to providing at least a portion of the therapy to the subject, and b) expression of GITR in a second sample following provision of the portion of the therapy, wherein a substantially modulated level of expression of GITR in the second sample, relative to the first sample, is an indication that the therapy is efficacious for inhibiting the autoimmune disorder or transplant rejection in the subject.

In another embodiment, the invention provides a method of high-throughput screening for test compounds capable of a cancer or proliferative disorder in a subject, the method comprising: a) combining a GITR protein, a specific factor which binds to a GITR protein, and a test compound; b) selecting one of the test compounds which modulates the binding GITR and the specific factor as compared to other test compounds; and c) correlating the amount of modulation of binding with the ability of the test compound to inhibit the cancer or proliferative disorder, wherein modulation of binding of GITR protein and the specific factor indicates that the test compound is capable of inhibiting the cancer or proliferative disorder.

In another embodiment, the invention provides a method of screening test compounds for inhibitors of a cancer or proliferative disorder in a subject, the method comprising the steps of: a) obtaining a sample comprising cells; b) contacting an aliquots of the sample with one of a plurality of test compounds; c) comparing the expression levels GITR in each of the aliquots; and d) selecting one of the test compounds which substantially modulated level of expression of a GITR expression in the aliquot containing that test compound, relative to other test compounds.

In another embodiment, the invention provides a method of treating a subject diagnosed with a cancer or proliferative disorder comprising administering a composition comprising an antagonist of a GITR polypeptide or a GITR polynucleotide, and a pharmaceutically acceptable carrier.

In another embodiment, the invention provides a method of treating a subject diagnosed with a cancer or proliferative disorder comprising administering a composition comprising an agonist of a GITR polypeptide or a GITR polynucleotide, and a pharmaceutically acceptable carrier.

In another embodiment, the invention provides a therapeutic target for the inhibition of a cancer or proliferative disorder, wherein the therapeutic target comprises a GITR marker gene.

In another embodiment, the invention provides a therapeutic target for the inhibition of a cancer or proliferative disorder, wherein the therapeutic target comprises a protein encoded by a GITR marker gene.

In another embodiment, the invention provides a kit for assessing the suitability of each of a plurality of compounds for inhibiting cancer or a proliferative disorder in a subject, the kit comprising: a) the plurality of compounds; and b) a reagent for assessing expression of GITR.

In another embodiment, the invention provides a kit for determining the long term prognosis in a subject having a cancer or proliferative disorder, wherein the kit comprises an antibody which specifically binds with a GITR protein.

In another embodiment, the invention provides a method of assessing the efficacy of a test compound for inhibiting an autoimmune disorder or transplant rejection in a subject, the method comprising the step of comparing: a) expression of a CD25⁺ differential marker in a first sample obtained from the subject, wherein the first sample is exposed to the test compound and wherein the CD25⁺ differential marker is a Cluster Type C or a Cluster Type D CD25⁺ differential marker (listed in Table I, or a homolog thereof), and b) expression of the same CD25⁺ differential marker in a second sample obtained from the subject, wherein the second sample is not exposed to the test compound, wherein a substantially increased level of expression of the CD25⁺ differential marker in the first sample, relative to the second sample, is an indication that the test compound is efficacious for inhibiting the autoimmune disorder or transplant rejection in the subject.

In a preferred embodiment, the invention provides the method wherein the CD25⁺ differential marker is a Cluster Type C CD25⁺ differential marker (listed in Table I, or a homolog thereof). wherein the CD25⁺ differential marker is a Cluster Type D CD25⁺ differential marker (listed in Table I, or a homolog thereof). In another preferred embodiment, the invention provides the method wherein the CD25⁺ differential marker is a Surface Receptor (listed in Table I, or a homolog thereof). In another preferred embodiment, the invention provides the method wherein the CD25⁺ differential marker is a Secreted marker (listed in Table I, or a homolog thereof). In another preferred embodiment, the invention provides the method wherein the first and second samples are portions of a single sample obtained from the subject. In another preferred embodiment, the invention provides the method wherein the level of expression in the first sample approximates the level of expression in a control sample.

In another preferred embodiment, the invention provides the method wherein the autoimmune disorder is selected from the group consisting of Multiple Sclerosis, Insulin-Dependent Diabetes Mellitus (Type I Diabetes), Inflammatory Bowel Disease Including Ulcerative Colitis, Crohns Disease (Regional Enteritis), Systemic Lupus Erythematosis, Vasculitis, Giant cell Arteritis, Polyarteritis Nodosa, Kawasaki's Disease, Allergic Granulomatosis, Aguitis, Psoriasis, Pemphigus Vulgaris, Pemphigus Foliaceus, Bullous Pemphigoid, Cicatricial Penphigoid, Dermatitis Herpetiformis, Acute Inflammatory Demylinating Polyradiculoneuropathy (Guillain-Barre Syndrome), Chronic Inflammatory Demyleinating Polyradiculoneuropathy, Peripheral Nerve Vasculitis, Lambert-Eaton Myasthenic Syndrome, Transverse Myelitis, Optic Neuritis, Neuromyelitis Optica, Autoimmune Gastritis, Hypophysitis, Polyglandular Autoimmune Endocrine Disease, Autoimmune Thyroiditis (Graves Disease, Hashimotos Thyroiditis), Autoimmune Disease of the Adrenal, Hypoparathyroidism, Insulin Autoimmune Syndrome, Autoimmune Uveitis, Episcleritis, Scleritis, Sjorgrens Syndrome, Behcets Syndrome, Retinal Vasculitis, Myasthenia Gravis, Idiopathic Inflammatory Myopathy, Polymyositis, Dermatomyositis, Autoimmune Myocardits, Dilated Cardiomyopathy, Autoimmune Diseases of the Reproductive Glands including Oophoritis Orchitis, Premature Ovarian Failure, Aplastic Anemia, Myelodysplastic Syndromes, Paroxysmal Nocturnal Hemoglobinuria, Red Cell Aplasia, Chronic Neutropenia, Autoimmune Thrombocytopenia, Autoimmune Hemolytic Anemia, Antiphospholipid Antibody Syndromes, Pernicious Anemia, Spontaneous Acquired Inhibitors of Coagulant Factors, Autoimmune Hepatitis, Primary Biliary Cirrhosis, Hepatitis C Associated Autoimmunity, Wegeners Granulomatosis, Sarcoidosis, Scleroderma, Asthma, Allergic Rhinitis, Metal Allergy, Contact Hypersensitivity, Drug Induced Autoimmunity, Immunoglobulin a Nephropathy, Membranous Nephropathy, Idiopathic Nephritic Syndrome, Mesangiocapillary Glomerulonephritis, Poststreptococcal Glomerulonephritis, Tubulointerstitial Nephritis, Goodpastures Syndrome, and Interstitial Cystitis.

In a highly preferred embodiment, the invention provides the method wherein the autoimmune disorder is selected from the group consisting of rheumatoid arthritis; systemic lupus erythematosis; psoriasis; multiple sclerosis; insulin-dependent diabetes mellitus (type I diabetes); inflammatory bowel disease including ulcerative colitis, Crohn's disease (regional enteritis); asthma; and allergic rhinitis. In another preferred embodiment, the invention provides the method wherein the samples are collected from blood.

In another embodiment, the invention provides a method of assessing the efficacy of a test compound for inhibiting an autoimmune disorder or transplant rejection in a subject, the method comprising the step of comparing: a) expression of a CD25⁺ differential marker in a first sample obtained from the subject, wherein the first sample is exposed to the test compound, wherein the CD25⁺ differential marker is a Cluster Type A or a Cluster Type B CD25⁺ differential marker (listed in Table I, or a homolog thereof), and b) expression of the same CD25⁺ differential marker in a second sample obtained from the subject, wherein the second sample is not exposed to the test compound, wherein a substantially decreased level of expression of the CD25⁺ differential marker in the first sample, relative to the second sample, is an indication that the test compound is efficacious for inhibiting an autoimmune disorder or transplant rejection in the subject.

In a preferred embodiment, the invention provides the method wherein the CD25⁺ differential marker is a Cluster Type B CD25⁺ differential marker (listed in Table I, or a homolog thereof). In another preferred embodiment, the invention provides the method wherein the CD25⁺ differential marker is a Cluster Type A CD25⁺ differential (listed in Table I, or a homolog thereof). In another preferred embodiment, the invention provides the method wherein the CD25⁺ differential marker is a Surface Receptor (listed in Table I, or a homolog thereof). In another preferred embodiment, the invention provides the method wherein the level of expression in the first sample approximates the level of expression in a control sample. In another preferred embodiment, the invention provides the method wherein the samples are collected from blood.

In another embodiment, the invention provides a method of assessing the efficacy of a therapy for inhibiting an autoimmune disorder or transplant rejection in a subject, the method comprising the steps of comparing: a) expression of a CD25⁺ differential marker in a first sample obtained from the subject prior to providing at least a portion of the therapy to the subject, wherein the CD25⁺ differential marker is a Cluster Type C or a Cluster Type D CD25⁺ differential marker (listed in Table I, or a homolog thereof), and b) expression of the CD25⁺ differential marker in a second sample following provision of the portion of the therapy, wherein a substantially increased level of expression of the CD25⁺ differential marker in the second sample, relative to the first sample, is an indication that the therapy is efficacious for inhibiting the autoimmune disorder or transplant rejection in the subject. In a preferred embodiment, the invention provides the method wherein the level of expression of the CD25⁺ differential marker in the second sample approximates the level of expression of the CD25⁺ differential marker in a control sample substantially free of said disorder.

In another embodiment, the invention provides a method of assessing the efficacy of a therapy for inhibiting an autoimmune disorder or transplant rejection in a subject, the method comprising the steps of comparing: a) expression of a CD25⁺ differential marker in a first sample obtained from the subject prior to providing at least a portion of the therapy to the subject, wherein the CD25⁺ differential marker is a Cluster Type A or a Cluster Type B CD25⁺ differential marker (listed in Table I, or a homolog thereof), and b) expression of the CD25⁺ differential marker in a second sample following provision of the portion of the therapy, wherein a substantially decreased level of expression of the CD25⁺ differential marker in the second sample, relative to the first sample, is an indication that the therapy is efficacious for inhibiting the autoimmune disorder or transplant rejection in the subject. In a preferred embodiment, the invention provides the method wherein the level of expression of the CD25⁺ differential marker in the second sample approximates the level of expression of the CD25⁺ differential marker in a control sample substantially free of said disorder.

In another embodiment, the invention provides a method of high-throughput screening for test compounds capable of modulating the activity of a panel of marker proteins encoded from a CD25⁺ differential marker (listed in Table I, or a homolog thereof), the method comprising: a) contacting the panel of proteins with a plurality of test compounds; b) detecting binding of one of the test compounds to the panel of proteins, relative to other test compounds; and c) correlating the amount of binding of the test compound to the panel of proteins with the ability of the test compound to modulate the activity of the protein, wherein binding indicates that the test compound is capable of modulating the activity of the protein.

In a preferred embodiment, the invention provides the method wherein the selected test compound prevents binding of the protein with a bioactive agent selected from the group consisting of naturally-occurring compounds, biomolecules, proteins, peptides, oligopeptides, polysaccharides, nucleotides and polynucleotides. In another preferred embodiment, the invention provides the method wherein the step of detecting binding is conducted by utilizing surface plasmon resonance. In another preferred embodiment, the invention provides the method wherein the test compounds are bioactive agents selected from the group consisting of naturally-occurring compounds, biomolecules, proteins, peptides, oligopeptides, polysaccharides, nucleotides and polynucleotides. In another preferred embodiment, the invention provides the method wherein the test compounds are small molecules. In yet another preferred embodiment, the invention provides the method wherein the CD25⁺ differential marker is a Cluster Type A or a Cluster Type B CD25⁺ differential marker (listed in Table I, or a homolog thereof). In yet another preferred embodiment, the invention provides the method wherein the CD25⁺ differential marker is a Cluster Type C or a Cluster Type D CD25⁺ differential marker (listed in Table I, or a homolog thereof). In another preferred embodiment, the invention provides the method wherein the marker is a Surface Receptor (listed in Table I, or a homolog thereof).

In another embodiment, the invention provides a method of high-throughput screening for test compounds capable of inhibiting an autoimmune disorder or transplant rejection, the method comprising: a) combining a CD25⁺ differential marker protein (listed in Table I, or a homolog thereof), a specific factor which binds to a protein, and a test compound; b) selecting one of the test compounds which modulates the binding of the CD25⁺ differential marker protein and the specific factor as compared to other test compounds; and c) correlating the amount of modulation of binding with the ability of the test compound to inhibit the autoimmune disorder or transplant rejection, wherein modulation of binding of the CD25⁺ differential marker protein and the specific factor indicates that the test compound is capable of inhibiting the autoimmune disorder or transplant rejection.

In a preferred embodiment, the invention provides the method wherein the step of selecting comprises detecting binding of one of the test compounds to the CD25⁺ differential marker protein. In another preferred embodiment, the invention provides the method wherein the step of selecting comprises detecting binding of one of the test compounds to the specific factor. In another preferred embodiment, the invention provides the method wherein the test compounds are small molecules. In another preferred embodiment, the invention provides the method wherein the test compounds are from a library selected from a group of libraries consisting of spatially addressable parallel solid phase or solution phase libraries or synthetic libraries made from deconvolution, ‘one-bead one-compound’ methods or by affinity chromatography selection. In another preferred embodiment, the invention provides the method wherein the test compounds are bioactive agents selected from the group consisting of naturally-occurring compounds, biomolecules, proteins, peptides, oligopeptides, polysaccharides, nucleotides and polynucleotides. In another preferred embodiment, the invention provides the method wherein the step of selecting comprises utilizing surface plasmon resonance. In another preferred embodiment, the invention provides the method wherein the CD25⁺ differential marker is a Cluster Type A or a Cluster Type B marker (listed in Table I, or a homolog thereof). In another preferred embodiment, the invention provides the method wherein the CD25⁺ differential marker is a Cluster Type C or a Cluster Type D marker (listed in Table I, or a homolog thereof).

In another embodiment, the invention provides a method of screening for a test compound capable of interfering with the binding of a protein encoded from a CD25⁺ differential marker (listed in Table I, or a homolog thereof), and a specific factor which binds to the protein, the method comprising: a) combining the protein, a test compound and the specific factor which binds to the protein; and b) determining the binding of the protein and the specific factor; and c) correlating the amount of binding with the ability of the test compound to interfere with binding, wherein a decrease in binding of the protein and the specific factor in the presence of the test compound as compared to the absence of the test compound indicates that the test compound is capable of inhibiting binding.

In a preferred embodiment, the invention provides the method wherein the specific factor is a substrate for the protein. In another preferred embodiment, the invention provides the method wherein the specific factor is a ligand for the protein. In another preferred embodiment, the invention provides the method wherein the specific factor is a polynucleotide. In another preferred embodiment, the invention provides the method wherein the protein is a Surface Receptor (listed in Table I, or a homolog thereof). In another preferred embodiment, the invention provides the method wherein the test compound is a small molecule. In another preferred embodiment, the invention provides the method wherein the test compound is selected from the group of libraries consisting of spatially addressable parallel solid phase or solution phase libraries or synthetic libraries made from deconvolution, ‘one-bead one-compound’ methods or by affinity chromatography selection. In another preferred embodiment, the invention provides the method wherein the test compound is also a bioactive agent selected from the group consisting of naturally-occurring compounds, biomolecules, proteins, peptides, oligopeptides, polysaccharides, nucleotides and polynucleotides. In another preferred embodiment, the invention provides the method wherein the test compound is a protein. In another preferred embodiment, the invention provides the method wherein the CD25⁺ differential marker is a Cluster Type A or Cluster Type B marker (listed in Table I, or a homolog thereof). In another preferred embodiment, the invention provides the method wherein the CD25⁺ differential marker is a Cluster Type C or Cluster Type C marker (listed in Table I, or a homolog thereof).

In another embodiment, the invention provides a method of screening test compounds for inhibitors of an autoimmune disorder or transplant rejection, the method comprising the steps of: a) obtaining a sample comprising cells; b) contacting an aliquots of the sample with one of a plurality of test compounds; c) comparing the expression levels of a CD25⁺ differential marker in each of the aliquots, wherein the CD25⁺ differential marker is selected from the group consisting of CD25⁺ differential markers (listed in Table I, or a homolog thereof); and d) selecting one of the test compounds which substantially decreased the level of expression of a Cluster Type A or Cluster Type B CD25⁺ differential marker or which substantially increased level of expression of a Cluster Type C or Cluster Type D CD25⁺ differential marker, in the aliquot containing that test compound, relative to other test compounds.

In a preferred embodiment, the invention provides the method wherein the test compounds are small molecules selected from the group of libraries consisting of spatially addressable parallel solid phase or solution phase libraries or synthetic libraries made from deconvolution, ‘one-bead one-compound’ methods or by affinity chromatography selection. In another preferred embodiment, the invention provides the method wherein the test compounds are bioactive agents selected from the group consisting of proteins, oligopeptides, polysaccharides and polynucleotides. In another preferred embodiment, the invention provides the method wherein the test compounds are proteins. In another preferred embodiment, the invention provides the method wherein the selected test compound induces an expression level in the CD25⁺ differential marker that approximates a normal level of expression in a sample substantially free of an autoimmune disorder. In another preferred embodiment, the invention provides the method wherein the sample is collected from a subject with an autoimmune disorder.

In another embodiment, the invention provides a method of determining the severity of an autoimmune disorder or transplant rejection in a subject, the method comprising the step of comparing: a) a level of expression of one or more CD25⁺ differential markers (listed in Table I, or a homolog thereof), in a sample from the subject; and b) a normal level of expression of the CD25⁺ differential marker in a control sample, wherein an abnormal level of expression of the one or more CD25⁺ differential markers in the sample from the subject relative to the normal levels is an indication that the subject is suffering from a severe autoimmune disorder or transplant rejection.

In a preferred embodiment, the invention provides the method wherein the CD25⁺ differential marker corresponds to a transcribed polynucleotide or a portion thereof. In another preferred embodiment, the invention provides the method wherein the sample is collected from blood. In another preferred embodiment, the invention provides the method wherein the control sample is collected from tissue substantially free of the autoimmune disorder and the abnormal increase is a factor of at least about 2. In another preferred embodiment, the invention provides the method wherein the presence of the protein is detected using a antibody or fragments thereof which specifically binds to the protein. In another preferred embodiment, the invention provides the method wherein the level of expression of the CD25⁺ differential marker in the sample is assessed by detecting the presence in the sample of a transcribed polynucleotide or portion thereof, wherein the transcribed polynucleotide comprises the CD25⁺ differential marker. In another preferred embodiment, the invention provides the method wherein the transcribed polynucleotide is a mRNA. In another preferred embodiment, the invention provides the method wherein the transcribed polynucleotide is a cDNA. In another preferred embodiment, the invention provides the method wherein the level of expression of the CD25⁺ differential marker in the sample is assessed by detecting the presence in the sample of a transcribed polynucleotide or a portion thereof which hybridizes with a labeled probe under stringent conditions, wherein the transcribed polynucleotide comprises the CD25⁺ differential marker.

In another embodiment, the invention provides a method of treating a subject diagnosed with an autoimmune disorder or transplant rejection comprising administering a therapeutically acceptable amount of a composition comprising a CD25⁺ differential marker polypeptide and a pharmaceutically acceptable carrier.

In another embodiment, the invention provides a method of treating a subject diagnosed with an autoimmune disorder or transplant rejection comprising administering a therapeutically acceptable amount of a composition comprising a CD25⁺ differential marker polynucleotide, a delivery vehicle and a pharmaceutically acceptable carrier.

In another embodiment, the invention provides a method of modulating a level of expression of a CD25⁺ differential marker (listed in Table I, or a homolog thereof), the method comprising providing to cells of a subject an antisense oligonucleotide complementary to a polynucleotide corresponding to the CD25⁺ differential marker.

In another embodiment, the invention provides a method of modulating a level of expression of a CD25⁺ differential marker (listed in Table I, or a homolog thereof), the method comprising providing to cells of a subject a protein corresponding to the CD25⁺ differential marker.

In a preferred embodiment, the invention provides the method wherein the protein is provided to the cells by providing a vector comprising a polynucleotide encoding the CD25⁺ differential marker protein.

In another embodiment, the invention provides a method of modulating a level of expression of a CD25⁺ differential marker (listed in Table I, or a homolog thereof), the method comprising providing to cells of a subject an antibody which specifically binds to the CD25⁺ differential marker protein (listed in Table I, or a homolog thereof). In a preferred embodiment, the invention provides the method wherein the method further comprises a therapeutic moiety conjugated to the antibody.

In another embodiment, the invention provides a method of localizing a therapeutic moiety to tissue having an autoimmune disorder or transplant rejection comprising: 1) linking a therapeutic agent to a binding partner of a CD25⁺ differential marker; and 2) administering to a subject in need of treatment, the therapeutic moiety linked to the binding partner.

In another embodiment, the invention provides a method of localizing a therapeutic moiety to tissue having an autoimmune disorder or transplant rejection comprising exposing the tissue to an antibody which is specific to a protein encoded from a CD25⁺ differential marker which is a Surface Receptor (listed in Table I, or a homolog thereof).

In another embodiment, the invention provides a method of localizing a therapeutic moiety to a tissue having an autoimmune disorder or transplant rejection comprising exposing the tissue to a plurality of antibodies which are each specific to a protein encoded from a CD25⁺ differential marker which is a Surface Receptor (listed in Table I, or a homolog thereof).

In another embodiment, the invention provides a biochip comprising at least 5 or more CD25⁺ differential markers (listed in Table I, or a homolog thereof), wherein the biochip is utilized in high-throughput screening assays for inhibition an autoimmune disorder or transplant rejection. In a preferred embodiment, the invention provides the method wherein biochip of claim 105, wherein the CD25⁺ differential markers are selected for subjects suspected of having rheumatoid arthritis. In a preferred embodiment, the invention provides the method wherein the CD25⁺ differential markers are selected for subjects having been diagnosed with an autoimmune disorder.

In another embodiment, the invention provides a composition capable of modulating an autoimmune disorder in a subject, the composition comprising one or more proteins encoded from a CD25⁺ differential marker (listed in Table I, or a homolog thereof) and a pharmaceutically acceptable carrier.

In another embodiment, the invention provides a composition capable of inhibiting a transplant rejection in a subject, the composition comprising one or more proteins encoded from a CD25⁺ differential marker (listed in Table I, or a homolog thereof) and a pharmaceutically acceptable carrier.

In another embodiment, the invention provides a therapeutic target for the inhibition of an autoimmune disorder or transplant rejection, wherein the therapeutic target comprises a CD25⁺ differential marker gene (listed in Table I, or a homolog thereof).

In another embodiment, the invention provides a therapeutic target for the inhibition of an autoimmune disorder or transplant rejection, wherein the therapeutic target comprises a protein encoded by a CD25⁺ differential marker (listed in Table I, or a homolog thereof). In a preferred embodiment, the invention provides the target the CD25⁺ differential marker is a Cluster Type A or Cluster Type B marker (listed in Table I, or a homolog thereof). In a preferred embodiment, the invention provides the target the CD25⁺ differential marker is a Cluster Type C or Cluster Type B marker (listed in Table I, or a homolog thereof).

In another embodiment, the invention provides a kit for determining the long term prognosis in a subject having an autoimmune disorder or transplant rejection, the kit comprising a polynucleotide probe wherein the probe specifically binds to a transcribed polynucleotide corresponding to a CD25⁺ differential marker (listed in Table I, or a homolog thereof). In a preferred embodiment, the invention provides the kit wherein the CD25⁺ differential marker is a Cluster Type A or a Cluster Type B marker (listed in Table I, or a homolog thereof). In another preferred embodiment, the invention provides the kit wherein the CD25⁺ differential marker is a Cluster Type C or a Cluster Type D marker (listed in Table I, or a homolog thereof).

In another embodiment, the invention provides a kit for assessing the suitability of each of a plurality of compounds for inhibiting an autoimmune disorder or transplant rejection in a subject, the kit comprising: a) the plurality of compounds; and b) a reagent for assessing expression of a CD25⁺ differential marker (listed in Table I, or a homolog thereof).

In another embodiment, the invention provides a kit for determining the long term prognosis in a subject having an autoimmune disorder or transplant rejection, wherein the kit comprises an antibody which specifically binds with a protein corresponding to a CD25⁺ differential marker (listed in Table I, or a homolog thereof). In a preferred embodiment, the invention provides the kit wherein the CD25⁺ differential marker is a Cluster Type C or a Cluster Type D marker (listed in Table I, or a homolog thereof). In another preferred embodiment, the invention provides the kit wherein CD25⁺ differential marker is a Surface Receptor (listed in Table I, or a homolog thereof).

In another embodiment, the invention provides a kit comprising a biochip and a computer readable medium, wherein the biochip comprises at least 5 CD25⁺ differential markers (listed in Table I, or a homolog thereof) and wherein the computer readable medium contains the same CD25⁺ differential markers in computer readable form.

In another embodiment, the invention provides a method of assessing the efficacy of a test compound for inhibiting a cancer or proliferative disorder in a subject, the method comprising the step of comparing: a) expression of one or more CD25⁺ differential marker (listed in Table I, or a homolog thereof) in a first sample obtained from the subject, wherein the first sample is exposed to the test compound, and b) expression of the same CD25⁺ differential marker (listed in Table I, or a homolog thereof) in a second sample obtained from the subject, wherein the second sample is not exposed to the test compound, wherein a substantially modulated level of expression of the CD25⁺ differential marker in the first sample, relative to the second sample, is an indication that the test compound is efficacious for inhibiting the cancer in the subject.

In another embodiment, the invention provides a method of assessing the efficacy of a therapy for inhibiting a cancer or proliferative disorder in a subject, the method comprising the steps of comparing: a) expression of one or more CD25⁺ differential marker (listed in Table I, or a homolog thereof) in a first sample obtained from the subject prior to providing at least a portion of the therapy to the subject, and b) expression of the same CD25⁺ differential marker(s) in a second sample following provision of the portion of the therapy, wherein a substantially modulated level of expression of the CD25⁺ differential marker in the second sample, relative to the first sample, is an indication that the therapy is efficacious for inhibiting the autoimmune disorder or transplant rejection in the subject.

In another embodiment, the invention provides a method of high-throughput screening for test compounds capable of a cancer or proliferative disorder in a subject, the method comprising: a) combining a D25⁺differential marker protein (listed in Table I, or a homolog thereof), a specific factor which binds to theCD25⁺ differential marker protein, and a test compound; b) selecting one of the test compounds which modulates the binding CD25⁺ differential marker protein and the specific factor as compared to other test compounds; and c) correlating the amount of modulation of binding with the ability of the test compound to inhibit the cancer or proliferative disorder, wherein modulation of binding of the CD25⁺ differential marker protein and the specific factor indicates that the test compound is capable of inhibiting the cancer or proliferative disorder.

In another embodiment, the invention provides a method of screening test compounds for inhibitors of a cancer or proliferative disorder in a subject, the method comprising the steps of: a) obtaining a sample comprising cells; b) contacting an aliquots of the sample with one of a plurality of test compounds; c) comparing the expression levels,one or more CD25⁺ differential marker (listed in Table I, or a homolog thereof) in each of the aliquots; and d) selecting one of the test compounds which substantially modulated level of expression of the CD25⁺ differential marker expression in the aliquot containing that test compound, relative to other test compounds.

In another embodiment, the invention provides a method of treating a subject diagnosed with a cancer or proliferative disorder comprising administering a composition comprising an antagonist of a CD25⁺ differential marker (listed in Table I, or a homolog thereof) polypeptide and a pharmaceutically acceptable carrier.

In another embodiment, the invention provides a method of treating a subject diagnosed with a cancer or proliferative disorder comprising administering a composition comprising an antagonist of a CD25⁺ differential marker (listed in Table I, or a homolog thereof) polynucleotide and a pharmaceutically acceptable carrier.

In another embodiment, the invention provides a method of treating a subject diagnosed with a cancer or proliferative disorder comprising administering a composition comprising an agonist of a CD25⁺ differential marker (listed in Table I, or a homolog thereof) polypeptide and a pharmaceutically acceptable carrier.

In another embodiment, the invention provides a method of treating a subject diagnosed with a cancer or proliferative disorder comprising administering a composition comprising an agonist of a CD25⁺ differential marker (listed in Table I, or a homolog thereof) polynucleotide and a pharmaceutically acceptable carrier.

In another embodiment, the invention provides a therapeutic target for the inhibition of a cancer or proliferative disorder, wherein the therapeutic target comprises a CD25⁺ differential marker gene (listed in Table I, or a homolog thereof).

In another embodiment, the invention provides a therapeutic target for the inhibition of a cancer or proliferative disorder, wherein the therapeutic target comprises a protein encoded by a CD25⁺ differential marker gene (listed in Table I, or a homolog thereof).

In another embodiment, the invention provides a kit for assessing the suitability of each of a plurality of compounds for inhibiting cancer or a proliferative disorder in a subject, the kit comprising: a) the plurality of compounds; and b) a reagent for assessing expression of CD25⁺ differential marker (listed in Table I, or a homolog thereof).

In another embodiment, the invention provides a kit for determining the long term prognosis in a subject having a cancer or proliferative disorder, wherein the kit comprises an antibody which specifically binds with a CD25⁺ differential marker (listed in Table I, or a homolog thereof) protein.

### BRIEF DESCRIPTION OF THE DRAWINGS

FIG. 1: Differential Expression in Resting CD25⁺ vs. CD25⁻ T cells. FIG. 1 illustrates genes which are differentially expressed between resting CD25⁺ and CD25⁻ T cells. Closed symbols are CD25⁺ and open symbols are CD25⁻. Squares represent values from the first of two replicate experiments and triangles represent values from the second of two replicate experiments. The x-axis displays mRNA frequency, expressed as number of mRNA molecules per million. The Affymetrix identifier is above each gene graph; GenBank accession number and common name are inside each gene graph.

FIGS. 2A-2B: Identification of Cell Surface Receptors Whose mRNA Expression is Elevated in CD25⁺ T cells. FIG. 2A illustrates gene expression values for the cell surface receptors GITR, OX-40, SCA-2 and CD103 in resting and induced cells. The top panel displays values in resting CD25⁺ and CD25⁻ T cells. The lighter shading represents CD25⁻ cells and the darker shading CD25⁺ cells. Triangles represent values for the first of two replicates and squares represent values for the second of two replicates. A filled black symbol at 0 value represents that the mRNA value was below the limit of detection. This was only true for the both reps of the CD25− cells for GITR. The x-axis represents mRNA frequency, in number of mRNA molecules per million. The lower panel displays mRNA expression values at 0, 12 or 48 hours after induction by anti-CD3 antibody. Filled symbols represent CD25⁺ cells and open symbols represent CD25⁻ cells. The x-axis is hours of anti-CD3 induction and the y-axis is mRNA frequency, expressed as number of mRNA molecules per million. The Affymetrix identifier is above each gene graph; the part of the identifier before the first underscore represents GenBank accession number. FIG. 2B illustrates gene expression values for the cell surface marker CTLA-4 in resting and induced cells. The top panel displays values in resting CD25⁺ and CD25− T cells. The unfilled symbols represent CD25− cells and the filled symbols CD25⁺ cells. Squares represent values for the first of two replicates and triangles represent values for the second of two replicates. The x-axis represents mRNA frequency, in number of mRNA molecules per million. The lower panel displays mRNA expression values at 0, 12 or 48 hours after induction by anti-CD3 antibody. Solid lines represent CD25⁺ cells and dashed lines represent CD25− cells. The x line marker indicates the first of two replicate experiments and the triangle line marker represents the second of two replicate experiments. The x-axis is hours of anti-CD3 induction and the y-axis is mRNA frequency, expressed as number of mRNA molecules per million. The Affymetrix identifier is above each gene graph; the part of the identifier before the first underscore represents GenBank accession number.

FIG. 3: Reversal of Suppression by anti-TNFRSF18 (GITR). The x-axis represents the number of CD4⁺CD25⁺ cells put into each assay well, and the y-axis represents cpm of ³H-thymidine incorporation into DNA, a measure of cellular proliferation. A constant number of responder CD4⁺CD25⁻ cells was put into each well. The open squares represent no antibody addition to the well; open diamonds represent addition of an irrelevant antibody; open circles represent addition of anti-GITR at 20 mg/ml; filled circles represent addition of anti-GITR at 2 mg/ml; the x symbols represent addition of anti-GITR at 0.2 mg/ml.

FIG. 4: Self Organizing Map (SOM) Clustering of Differential Expression in CD25⁺ vs. CD25⁻ T cells Before or After Anti-CD3 Activation. The x-axis is hours; the y-axis if normalized mRNA frequency, which involves a natural log transformation of absolute mRNA frequency values. This transformation has the effect of grouping genes together based on expression pattern over time, independent of expression magnitude. Each line represents a different gene. Solid lines are the values for CD25⁺ cells and dashed lines are for CD25⁻ cells.

FIGS. 5A-5D: Kinetic Profiles of Genes 3-fold Different in CD25⁺ vs. CD25⁻ in at Least One Timepoint. The genes populating the Self Organizing Map of FIG. 4 which met the 3-fold criterion are graphed out individually in FIG. 5 panels 5A-D. The x-axis is hours and the y-axis is absolute mRNA frequency, expressed as number of mRNA molecules per million. The Affymetrix identifier is above each gene graph; GenBank accession number and common name are inside each gene graph.

FIGS. 6A-6B: CD25⁺ Increase in Both Reps, No Fold Filter, Visual Inspection, Excludes Qualifiers>3F in Both Reps. Kinetic profiles of genes not meeting the 3-fold criterion for expression differences between CD25⁺ and CD25⁻ cells, but which are reproducibly differentially expressed between the two cellular populations. The genes populating the Self Organizing Map of FIG. 4 which did not meet the 3-fold criterion, but which were reproducibly differentially expressed between the two cellular populations, are graphed out individually in FIG. 6 panels A-B. The x-axis is hours and the y-axis is absolute mRNA frequency, expressed as number of mRNA molecules per million. The Affymetrix identifier is above each gene graph; GenBank accession number and common name are inside each gene graph.

FIG. 7: Comparison of Cell Surface Receptor Levels in CD4⁺CD25⁻ and CD4⁺CD25⁺ Cells in Resting and Activated States. FACS profiles of cell surface markers that were differentially expressed at the mRNA level between CD25⁺ and CD25⁻ cells. The left two panels represent Resting cells and the right two panels the cells after activation by plate-bound anti-CD3 ⁺IL-2 for 48 hours. The first and third panels are CD25⁻ cells and the second and fourth are CD25⁺ cells. Gene name is indicated on the left for each row of panels. The x-axis is fluorescence and the y-axis is the number of cells. The Control Ig is used to set the “gate” for cells positively staining with a given antibody (i.e. all cells fluorescing to at a value to the right of the gate are scored as positive for that antibody.) The percentage figures in each panel quantitate the percentage of cells scoring positive for binding to a given antibody. The numbers to the right of panels 1 and 2 and to the right of panels 3 and 4 are the Mean Fluorescence Index, which is a ratio of the mean fluorescence value for those cells scoring positive for a given antibody for the CD25⁺ cells to those cells scoring positive for a given antibody for the CD25⁻ cells.

FIG. 8: Results of CD103⁺CD25⁺ and CD103⁻CD25⁺ Cells Assayed in a Standard In vitro Suppression Assay. FIG. 8 illustrates suppressive bioactivity of CD103⁺ and CD103⁻ fractions of CD25⁺ cells. A constant number of 50,000 CD25⁻ responder cells were added to each well in the suppression assay. The x-axis is the number of CD25⁺CD103⁺, CD25⁺CD103⁻ or unfractionated CD25⁺ cells added to each well. The y-axis is the percent suppression of responder cell proliferation relative to a well in which no CD25⁺ cells were added.

FIGS. 9A-9E: Anti-GITR Antibody Reversal of Suppression of CD4⁺CD25⁺ cell Proliferation. Panel 9A is the suppression assay using CD25⁻ responder cells from Balb/c mice and CD25⁺ suppressor cells that were not pre-activated. The x-axis is the number of CD25⁺ cells added per well. The y-axis is percent suppression relative to a well in which no CD25⁺ cells were added. Panel 9B is the suppression assay using CD25⁻ responder cells from Balb/c mice and CD25⁺ suppressor cells that were pre-activated. Panel 9C is the suppression assay using CD25⁻ responder cells from HA T cell receptor transgenic mice (so that stimulation could be performed with anigen rather than with anti-CD3) and CD25⁺ suppressor cells that were not pre-activated. Panel 9D is the suppression assay using CD25⁻ responder cells from HA T cell receptor transgenic mice and CD25⁺ suppressor cells that were pre-activated. Panel 9E shows the results of a suppression assay which utilized CD8⁺cells as the responders. The x-axis shows the number of CD4⁺CD25⁺ cells added to each well. The y-axis shows cpm of ³H-thymidine. In panel 9E, filled squares represent well in which anti-GITR was added and open squares represent wells in which an irrelevant antibody was added. In panels 9A-D, open squares represent wells receiving no antibody; open diamonds represent wells receiving irrelevant antibody; filled circles represent well in which anti-GITR was added.

FIGS. 10A-B: CD4⁺CD25⁻ Cells Stimulated with Soluble anti-CD3 in the Presence of Either Anti-CD28 or Anti-GITR. FIG. 10 illustrates that anti-GITR does not provide a CD28-like costimulatory signal to CD4⁺CD25⁻ responders. CD25⁻ responder cells (50,000 per well) were activated to different concentrations of anti-CD3 in the presence of no antibody (open squares); irrelevant antibody (open diamonds); anti-GITR (closed circles) or anti-CD28 (open triangles). The x-axis is different concentraions of anti-CD3 and the y-axis is cpm of ³H-thymidine incorporation into DNA (a measure of cellular proliferation). The left panel shows the results of adding the antibodies at 10 mg/ml and the right panel at 2 mg/ml.

### DETAILED DESCRIPTION OF THE INVENTION

The present invention provides for the identification of novel targets and therapeutics for the intervention and prevention of autoimmune disorders. In particular, the present invention provides for the identification of novel therapeutic targets to be analyzed in high-throughput screening assays of test compounds capable of preventing, or treating an autoimmune disorder. The present invention further provides methods and compositions for the identification of novel targets for diagnosis, prognosis, therapeutic intervention and prevention of autoimmune disorders. In particular, the present invention provides the identification of novel targets which are CD25⁺ differential markers. The present invention provides methods of high-throughput screening for test compounds capable of modulating the activity or expression of proteins encoded by the novel targets. Moreover, the present invention provides methods that can be used to assess the efficacy of test compounds and therapies for the ability to inhibit an autoimmune disorder. Methods for determining the long term prognosis in a subject having an autoimmune disorder are also provided. The invention also provides novel methods for preventing transplant rejection. Further, the invention provides therapeutic intervention for cancer by providing methods and compositions related to CD25⁺ differential markers and suppressive T cells.

Definitions & Terms

To facilitate an understanding of the present invention, a number of terms and phrases are defined below:

As used herein, the term “CD25⁺ differential marker” or “marker” includes a polynucleotide or polypeptide molecule which is increased or decreased in quantity or activity in CD25⁺ T cells as compared to CD25⁻ T cells. In certain embodiments, the CD25⁺ differential markers of the invention include the markers listed in Table I, as well as homologs or isoforms thereof, particularly human homologs or human isoforms.

As used herein, the terms “polynucleotide,” “nucleic acid” and “oligonucleotide” are used interchangeably, and include polymeric forms of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, DNA, cDNA, genomic DNA, recombinant polynucleotides, branched polynucleotides, plasmids, vectors, isolated DNA of any sequence, isolated RNA of any sequence, nucleic acid probes, and primers. Polynucleotides of the invention may be naturally-occurring, synthetic, recombinant or any combination thereof. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine(C); guanine (G); thymine (T); and uracil (U) in place of guanine when the polynucleotide is RNA. Thus, the term “polynucleotide sequence” is the alphabetical representation of a polynucleotide molecule. This alphabetical representation can be inputted into databases in a computer and used for bioinformatics applications such as functional genomics and homology searching.

The term “isolated polynucleotide molecule” includes polynucleotide molecules which are separated from other polynucleotide molecules which are present in the natural source of the polynucleotide. For example, with regards to genomic DNA, the term “isolated” includes polynucleotide molecules which are separated from the chromosome with which the genomic DNA is naturally associated. Preferably, an “isolated” polynucleotide is free of sequences which naturally flank the polynucleotide (i.e., sequences located at the 5′ and 3′ ends of the polynucleotide of interest) in the genomic DNA of the organism from which the polynucleotide is derived. For example, in various embodiments, the isolated marker polynucleotide molecule of the invention, or polynucleotide molecule encoding a polypeptide marker of the invention, can contain less than about 5 kb, 4 kb, 3 kb, 2 kb, 1 kb, 0.5 kb or 0.1 kb of nucleotide sequences which naturally flank the polynucleotide molecule in genomic DNA of the cell from which the polynucleotide is derived. Moreover, an “isolated” polynucleotide molecule, such as a cDNA molecule, can be substantially free of other cellular material, or culture medium when produced by recombinant techniques, or substantially free of chemical precursors or other chemicals when chemically synthesized.

A “gene” includes a polynucleotide containing at least one open reading frame that is capable of encoding a particular polypeptide or protein after being transcribed and translated. Any of the polynucleotide sequences described herein may also be used to identify larger fragments or full-length coding sequences of the gene with which they are associated. Methods of isolating larger fragment sequences are known to those of skill in the art, some of which are described herein.

The term “noncoding region” includes 5′ and 3′ sequences which flank the coding region that are not translated into amino acids (i.e., also referred to as 5′ and 3′ untranslated regions).

As used herein, a “naturally-occurring” polynucleotide molecule includes for example an RNA or DNA molecule having a nucleotide sequence that occurs in nature (e.g., encodes a natural protein).

As used herein, the term, “transcribed” or “transcription” refers to the process by which genetic code information is transferred from one kind of nucleic acid to another, and refers in particular to the process by which a base sequence of mRNA is synthesized on a template of cDNA.

The term “polypeptide” includes a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

A “gene product” includes an amino acid sequence (e.g., peptide or polypeptide) generated when a gene is transcribed and translated.

As used herein, a marker “chimeric protein” or “fusion protein” comprises a marker polypeptide operatively linked to a non-marker polypeptide. A “marker polypeptide” includes a polypeptide having an amino acid sequence encoded by a CD25⁺ differential marker set forth in Table I, whereas a “non-marker polypeptide” includes a polypeptide having an amino acid sequence corresponding to a protein which is not substantially homologous to the marker protein, e.g., a protein which is different from marker protein and which is derived from the same or a different organism.

An “isolated” or “purified” protein or biologically active portion thereof is substantially free of cellular material or other contaminating proteins from the cell or tissue source from which the marker protein is derived, or substantially free from chemical precursors or other chemicals when chemically synthesized. The language “substantially free of cellular material” includes preparations of marker protein in which the protein is separated from cellular components of the cells from which it is isolated or recombinantly produced. In one embodiment, the language “substantially free of cellular material” includes preparations of marker protein having less than about 30% (by dry weight) of non-marker protein (also referred to herein as a “contaminating protein”), more preferably less than about 20% of non-marker protein, still more preferably less than about 10% of non-marker protein, and most preferably less than about 5% non-marker protein. When the marker protein or biologically active portion thereof is recombinantly produced, it is also preferably substantially free of culture medium (i.e., culture medium) represents less than about 20%, more preferably less than about 10%, and most preferably less than about 5% of the volume of the protein preparation.

The language “substantially free of chemical precursors or other chemicals” includes preparations of marker protein in which the protein is separated from chemical precursors or other chemicals which are involved in the synthesis of the protein. In one embodiment, the language “substantially free of chemical precursors or other chemicals” includes preparations of protein having less than about 30% (by dry weight) of chemical precursors or non-protein chemicals, more preferably less than about 20% chemical precursors or non-protein chemicals, still more preferably less than about 10% chemical precursors or non-protein chemicals, and most preferably less than about 5% chemical precursors or non-protein chemicals.

As used herein, a “biologically active portion” of a marker protein includes a fragment of a marker protein comprising amino acid sequences sufficiently homologous to or derived from the amino acid sequence of the marker protein, which include fewer amino acids than the full length marker proteins, and exhibit at least one activity of a marker protein. Typically, biologically active portions comprise a domain or motif with at least one activity of the marker protein. A biologically active portion of a marker protein can be a polypeptide which is, for example, 10, 25, 50, 100, 200 or more amino acids in length. Biologically active portions of a marker protein can be used as targets for developing agents which modulate a marker protein-mediated activity.

“Differentially” or “abnormally” expressed, as applied to a gene, includes the differential production of mRNA transcribed from a gene. A differentially or abnormally expressed gene may be overexpressed or underexpressed as compared to the expression level of a normal cell or control cell or CD25⁻ T cell. In one aspect, abnormal or differential expression refers to a level of expression that differs from normal levels of expression by one normal standard of deviation. In a preferred aspect, the differential is 2 times or higher or lower than the expression level detected in a control sample. The term “differentially-” or “abnormally-” expressed also includes nucleotide sequences in a cell or tissue which are not expressed where expressed in a normal cell or control cell or CD25⁻ T cell. In certain embodiments of the invention, the control cell is a CD25⁻ T cell. In certain embodiments differential expression is compared between a CD25⁺ T cell and a CD25⁻ T cell or populations thereof. In certain embodiments the normal cell or sample or control cell or sample is substantially free of an autoimmune disease or cancer.

As used herein, the term “aberrant” includes a marker expression or activity which deviates from the normal marker expression or activity. Aberrant expression or activity includes increased or decreased expression or activity, as well as expression or activity which does not follow the normal developmental pattern of expression or the subcellular pattern of expression. For example, aberrant marker expression or activity is intended to include the cases in which a mutation in the marker gene causes the marker gene to be under-expressed or over-expressed and situations in which such mutations result in a non-functional marker protein or a protein which does not function in a normal fashion (e.g., a protein which does not interact with a marker ligand or one which interacts with a non marker protein ligand.) In certain embodiments the normal cell or sample or control cell or sample is substantially free of an autoimmune disease or cancer.

As used herein, the term “modulation” includes, in its various grammatical forms (e.g., “modulated”, “modulation”, “modulating”, etc.), up-regulation, induction, stimulation, potentiation, and/or relief of inhibition, as well as inhibition and/or down-regulation or suppression.

A “probe” when used in the context of polynucleotide manipulation includes an oligonucleotide that is provided as a reagent to detect a target present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” includes a short polynucleotide, generally with a free 3′-OH group that binds to a target or “template” present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or “set or primers” consisting of “upstream” and a “downstream” primer, and a catalyst of polymerization, such as a DNA polymerase, and typically a thermally-stable polymerase enzyme. Methods for PCR are well known in the art, and are taught, for example, in MacPherson et al., IRL Press at Oxford University Press (1991). All processes of producing replicate copies of a polynucleotide, such as PCR or gene cloning, are collectively referred to herein as “replication”. A primer can also be used as a probe in hybridization reactions, such as Southern or Northern blot analyses (see, e.g., Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

The term “cDNAs” includes complementary DNA, that is mRNA molecules present in a cell or organism made into cDNA with an enzyme such as reverse transcriptase. A “cDNA library” includes a collection of mRNA molecules present in a cell or organism, converted into cDNA molecules with the enzyme reverse transcriptase, then inserted into “vectors” (other DNA molecules that can continue to replicate after addition of foreign DNA). Exemplary vectors for libraries include bacteriophage, viruses that infect bacteria (e.g., lambda phage). The library can then be probed for the specific cDNA (and thus mRNA) of interest.

A “gene delivery vehicle” includes a molecule that is capable of inserting one or more polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, viruses and viral vectors, such as baculovirus, adenovirus, and retrovirus, bacteriophage, cosmid, plasmid, fungal vector and other recombination vehicles typically used in the art which have been described for replication and/or expression in a variety of eukaryotic and prokaryotic hosts. The gene delivery vehicles may be used for replication of the inserted polynucleotide, gene therapy as well as for simply polypeptide and protein expression.

A “vector” includes a self-replicating nucleic acid molecule that transfers an inserted polynucleotide into and/or between host cells. The term is intended to include vectors that function primarily for insertion of a nucleic acid molecule into a cell, replication vectors that function primarily for the replication of nucleic acid and expression vectors that function for transcription and/or translation of the DNA or RNA. Also intended are vectors that provide more than one of the above function.

A “host cell” is intended to include any individual cell or cell culture which can be or has been a recipient for vectors or for the incorporation of exogenous polynucleotides and/or polypeptides. It also is intended to include progeny of a single cell. The progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, insect cells, animal cells, and mammalian cells, including but not limited to murine, rat, simian or human cells.

The term “genetically modified” includes a cell containing and/or expressing a foreign or exogenous gene or polynucleotide sequence which in turn modifies the genotype or phenotype of the cell or its progeny. This term includes any addition, deletion, or disruption to a cell's endogenous nucleotides.

As used herein, “expression” includes the process by which polynucleotides are transcribed into RNA and translated into polypeptides or proteins. If the polynucleotide is derived from genomic DNA, expression may include splicing of the RNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

As used herein, a “test sample” includes a biological sample obtained from a subject of interest. For example, a test sample can be a biological fluid (e.g., blood, T cells,), cell sample, or tissue (e.g., lymph node tissue).

As used herein, “hybridization” includes a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. The stringency of a hybridization reaction includes the difficulty with which any two nucleic acid molecules will hybridize to one another. The present invention also includes polynucleotides capable of hybridizing under reduced stringency conditions, more preferably stringent conditions, and most preferably highly stringent conditions, to polynucleotides described herein. Examples of stringency conditions are shown in Table A below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE A
Stringency Conditions

| | Poly- | | | |
|---|---|---|---|---|
| Stringency | nucleotide | Hybrid | Hybridization Temperature | Wash Temperature |
| Condition | Hybrid | Length (bp)¹ | and Buffer^{H} | and Buffer^{H} |
| A | DNA DNA | >50 | 65° C.; 1xSSC -or- | 65° C., 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C., 1xSSC, 50% formamide | |
| D | DNA:RNA | <50 | T_{D}*, 1xSSC | T_{D}*; 1xSSC |
| E | RNA.RNA | >50 | 70° C.; 1xSSC -or- | 70° C.; 0.3xSSC |
| | | | 50° C.; 1xSSC, 50% formamide | |
| F | RNA.RNA | <50 | T_{F}*; 1xSSC | T_{f}*, 1xSSC |
| G | DNA:DNA | >50 | 65° C., 4xSSC -or- | 65° C.; 1xSSC |
| | | | 42° C.; 4xSSC, 50% formamide | |
| H | DNA.DNA | <50 | T_{H}*; 4xSSC | T_{H}*, 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| M | DNA:DNA | >50 | 50° C.; 4xSSC -or- | 50° C.; 2xSSC |
| | | | 40° C.; 6xSSC, 50% formamide | |
| N | DNA:DNA | <50 | T_{N}*; 6xSSC | T_{N}*; 6xSSC |
| O | DNA.RNA | >50 | 55° C.; 4xSSC -or- | 55° C.; 2xSSC |
| | | | 42° C.; 6xSSC, 50% formamide | |
| P | DNA:RNA | <50 | T_{P}*; 6xSSC | T_{P}*; 6xSSC |
| Q | RNA:RNA | >50 | 60° C.; 4xSSC -or- | 60° C.; 2xSSC |
| | | | 45° C.; 6xSSC, 50% formamide | |
| R | RNA.RNA | <50 | T_{R}*, 4xSSC | T_{R}*; 4xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{H}SSPE (1xSSPE is 0.15 M NaCl, 10 mM NaH₂PO₄, and 1.25 mM EDTA, pH 7 4) can be substituted for SSC (1xSSC is 0 15 M NaCl and 15 mM sodium citrate) in the hybridization and wash buffers, washes are performed for 15 minutes after hybridization is complete. | | | | |
| T_{B}*-T_{R}* The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A ⁺T bases) ⁺4(# of G ⁺C | | | | |
| # bases). For hybrids between 18 and 49 base pairs in length, Tₘ(° C.) = 81.5 ⁺16.6 (log₁₀Na⁺) ⁺0.41(% G⁺C) - (600/N), where N is the number of bases in the hybrid, and Na⁺ is the concentration of sodium ions in the hybridization buffer (Na⁺ for 1xSSC = 0 165 M) | | | | |

Additional examples of stringency conditions for polynucleotide hybridization are provided in Sambrook, J., E F. Fritsch, and T Maniatis, 1989, Molecular Cloning A Laboratory Manual, Cold Spring Harbor Laboratory Press, Cold Spring Harbor, NY, chapters 9 and 11, and Current Protocols in Molecular Biology, 1995, F. M. Ausubel et al., eds., John Wiley & Sons, Inc., sections 2.10 and 6.3-6.4, incorporated herein by reference

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to hydrogen bond with each other, according to generally accepted base-pairing rules.

An “antibody” includes an immunoglobulin molecule capable of binding an epitope present on an antigen. As used herein, the term encompasses not only intact immunoglobulin molecules such as monoclonal and polyclonal antibodies, but also anti-idotypic antibodies, mutants, fragments, fusion proteins, bi-specific antibodies, humanized proteins, and modifications of the immunoglobulin molecule that comprises an antigen recognition site of the required specificity.

As used herein, the term “autoimmune disorder” includes but is not limited to Multiple Sclerosis, Insulin-Dependent Diabetes Mellitus (Type I Diabetes), Inflammatory Bowel Disease Including Ulcerative Colitis and Crohns Disease (Regional Enteritis), Systemic Lupus Erythematosis, Vasculitis, Giant cell Arteritis, Polyarteritis Nodosa, Kawasaki's Disease, Allergic Granulomatosis, Agiitis, Psoriasis, Pemphigus Vulgaris, Pemphigus Foliaceus, Bullous Pemphigoid, Cicatricial Penphigoid, Dermatitis Herpetiformis, Acute Inflammatory Demylinating Polyradiculoneuropathy (Guillain-Barre Syndrome), Chronic Inflammatory Demyleinating Polyradiculoneuropathy, Peripheral Nerve Vasculitis, Lambert-Eaton Myasthenic Syndrome, Transverse Myelitis, Optic Neuritis, Neuromyelitis Optica, Autoimmune Gastritis, Hypophysitis, Polyglandular Autoimmune Endocrine Disease, Autoimmune Thyroiditis (Graves Disease, Hashimotos Thyroiditis), Autoimmune Disease of the Adrenal, Hypoparathyroidism, Insulin Autoimmune Syndrome, Autoimmune Uveitis, Episcleritis, Scleritis, Sjorgrens Syndrome, Behcets Syndrome, Retinal Vasculitis, Myasthenia Gravis, Idiopathic Inflammatory Myopathy, Polymyositis, Dermatomyositis, Autoimmune Myocardits, Dilated Cardiomyopathy, Autoimmune Diseases of the Reproductive Glands including Oophoritis Orchitis, Premature Ovarian Failure, Aplastic Anemia, Myelodysplastic Syndromes, Paroxysmal Nocturnal Hemoglobinuria, Red Cell Aplasia, Chronic Neutropenia, Autoimmune Thrombocytopenia, Autoimmune Hemolytic Anemia, Antiphospholipid Antibody Syndromes, Pernicious Anemia, Spontaneous Acquired Inhibitors of Coagulant Factors, Autoimmune Hepatitis, Primary Biliary Cirrhosis, Hepatitis C Associated Autoimmunity, Wegeners Granulomatosis, Sarcoidosis, Scleroderma, Asthma, Allergic Rhinitis, Metal Allergy, Contact Hypersensitivity, Drug Induced Auto immunity, Immunoglobulin a Nephropathy, Membranous Nephropathy, Idiopathic Nephritic Syndrome, Mesangiocapillary Glomerulonephritis, Poststreptococcal Glomerulonephritis, Tubulointerstitial Nephritis, Goodpastures Syndrome, and Interstitial Cystitis.

As used herein “cancer” or “cancer or proliferative disease” includes but is not limited to renal cancer, melanoma, breast cancer, lymphoma, or multiple myeloma.

As used herein “transplant rejection” includes immune responses following transplantation of an organ or tissue (including but not limited to kidney, heart, skin, liver, pancreas, small bowel, or lung). Generally, organ or tissue transplant relates to the transfer of an organ or tissue from one subject to a second subject. In many cases, the major risk involved with transplantation is rejection of the newly transplanted organ or tissue in the recipient subject. Transplant rejection is well known in the art, and such definitions as used in the art are within the scope of the present invention.

As used herein, the term “normal” refers to cells, tissues or other such samples taken either pre-disorder or from a subject who has not suffered the autoimmune disorder or cancer, or from a cell, tissue or sample that is substantially free of an autoimmune disease or cancer. Control samples of the present invention are taken from normal samples or from CD25⁻ T cell samples. As used herein, a “control level of expression” refers to the level of expression associated with control samples thereof.

As used herein, the term “therapeutic target” refers to a polypeptide or polynucleotide or a biochemical complex, e.g, an enzyme-substrate complex, a receptor-ligand complex or a protein-antibody complex, which is the subject of diagnostic manipulation for treating or preventing injury caused by an autoimmune disease, transplant rejection, cancer, or proliferative disease. In the present invention, the therapeutic targets are the subject of manipulation in assays or treatments for inhibiting autoimmune disorder. In other embodiments, the therapeutic targets are the subject of manipulation in assays or treatments for inhibiting cancer. More specifically, the therapeutic targets of the invention may include transcription factors and polynucleotides, cell surface receptors and their ligands, as well as molecules involved in calcium regulation or metabolism, carbohydrate metabolism, cell cycle regulation, cytoskeleton, lipid metabolism, general metabolism, nucleotide metabolism, protein metabolism, or signaling. The therapeutic targets of the invention may also include a molecule that is a small G protein, a secreted protein, a kinase, or a molecule with unknown function. In certain embodiments, the present invention is directed to orphan receptors where the cognate ligand has yet to be identified.

As used herein, the term “panel of markers” includes a group of markers, the quantity or activity of each member of which is correlated with the incidence or risk of incidence of an autoimmune disorder. A panel of markers comprises 5 or more CD25⁺ differential markers. A panel may also comprise 5-15, 15-35, 35-50, 50-100, or more than 100 CD25⁺ differential markers. In certain embodiments, a panel of markers may include only those markers which are abnormally increased or abnormally decreased in quantity or activity in subjects having or suspected of having an autoimmune disorder or cancer. In a preferred embodiment, the panel of markers comprises at least 5 markers, preferably 10, more preferably 15 of the panel markers listed in Table I. In other embodiments, a panel of markers may include only those markers useful for a specific autoimmune disorder or a specific cancer.

Various aspects of the invention are described in further detail in the following subsections:

The subsections below describe in more detail the present invention. The use of subsections is not meant to limit the invention; subsections may apply to any aspect of the invention.

CD25⁺ Differential Markers

As shown in the Examples below, expression levels are recorded in CD25⁺ and CD25− T cells. While animal subjects are provided in the present invention for a more detailed analysis of an autoimmune disorder, it is well-appreciated in the art that expression levels of genes in animal models reflect expression levels from human subjects as well. It is specifically intended by the invention and understood that the CD25⁺ differential markers of the invention also specifically encompass human homologs of the CD25⁺ differential markers listed in Table I. Markers from other organisms may also be useful as animal models for the study of autoimmune disorders and for drug evaluation. Markers from other organisms may be obtained using the techniques outlined below.

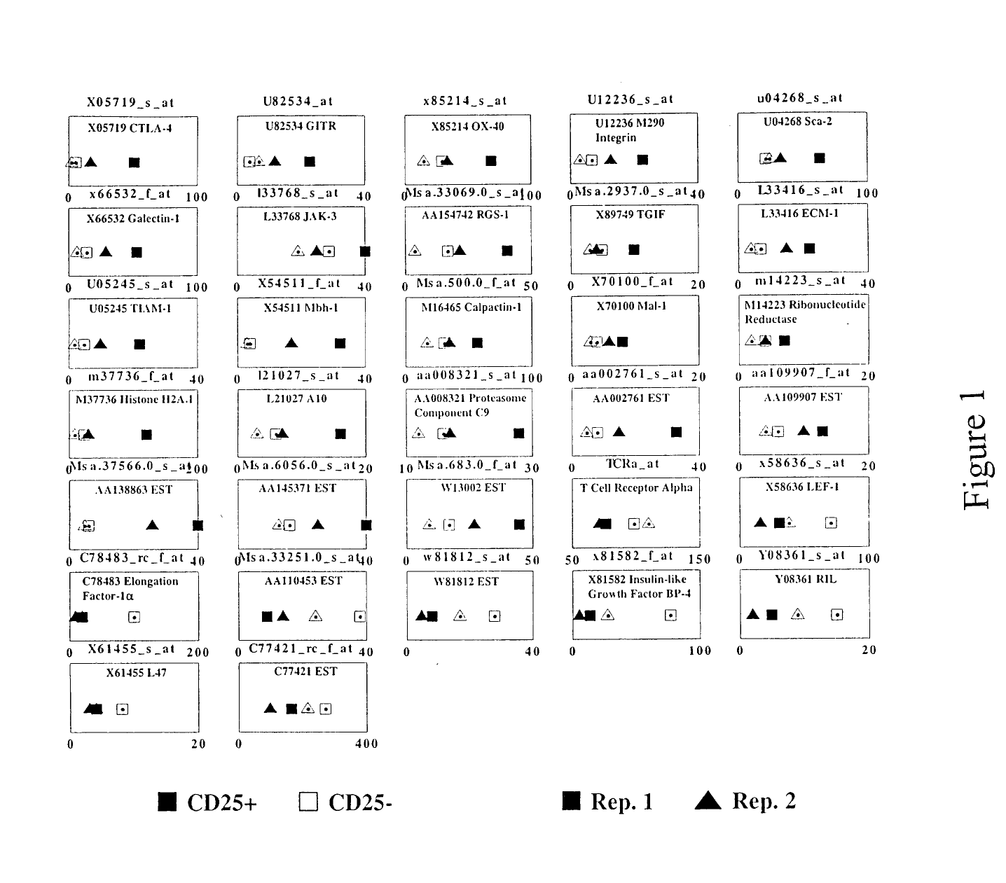

In one aspect, the present invention is based on the identification of a number of genetic markers, set forth in Table I, which are differentially expressed in CD25⁺ T cell samples, relative to CD25⁻ T cell samples. These markers may in turn be components of novel therapeutic targets for intervention in autoimmune disorders. Further, these markers may be useful in the treatment of cancer or proliferative disorders. The expression levels of genes that were differentially expressed between CD25⁺ and CD25⁻ T cells at different time points either before activation or after activation, are set forth in FIGS. 1-10 and Table I. In general, Table I provides CD25⁺ differential markers which are expressed at abnormally increased or decreased levels in CD25⁺ T cells as compared to CD25⁻ T cells. These genes are a component in novel therapeutic targets for the treatment and prevention of autoimmune disorders and cancer provided herein.

Genes listed in Table I were found to be differentially expressed in the CD25⁺ T cells as opposed to CD25⁻ T cells. These genes and their corresponding gene products (and detectable fragments thereof) are hereinafter known as CD25⁺ differential markers. Of the CD25⁺ differential markers identified, four classes of markers were identified. These four classes of markers are listed in Table I as Cluster Types A-D.

Cluster Type A represents CD25⁺ differential markers which show increased expression in CD25⁻ T cells at rest as compared to CD25⁺ T cells expression of the same marker but for which expression drops to levels similar or substantially similar to CD25⁺ T cells after activation, for example, at later time points such as 12 or 48 hour after activation.

Cluster Type B represents CD25⁺ differential markers which show transient increased expression in CD25⁻ T cells after activation as compared to CD25⁺ T cell expression of the same marker, for example expression may be increased at 12 hours after activation, but drop to levels similar or substantially similar to CD25⁺ T cell expression of the same marker at a later time point such as 48 hours post activation.

Cluster Type C represents CD25⁺ differential markers which show transient increased expression levels in CD25⁺ T cells after activation as compared to expression of the same marker in CD25⁻ T cells, but which are similar or substantially similar to CD25⁻ T cell expression of the same marker at later time point such as 48 hours post-activation.

Cluster Type D represents CD25⁺ differential markers which show a sustained increased expression in CD25⁺ T cells after activation as compared to the same marker in a CD25⁻ T cells, for example a Cluster Type D marker may show increased expression at 12 and 48 hours post-expression.

TABLE I
CD25⁺Differential Markers

| | Accession | | Cluster |
|---|---|---|---|
| Functional Category | Number | Common Name | Type |
| Calcium | M37761 | Calcyclin | C |
| | M96823 | Nucleobindin | C |
| | M16465 | Calpactin I | C |
| Carbohydrate Metab. | L25885 | GM2/GD2 Synth. | B |
| | AA174394 | Phos.inos.glyc. | C |
| | W13002 | LGALS1 | D |
| Cell Cycle | AA138777 | GADD45-g | D |
| | X58708 | Cyclin B | D |
| Surface Receptor | L38971 | ITM-2 | B |
| | M77167 | TCRa | B |
| | U12236 | CD103 | C |
| | K02891 | IL2Ra | C |
| | M28052 | IL2Rb | C |
| | M80481 | GIR | C |
| | L29441 | TMS-1 | C |
| | AA027619 | Ly6C | C |
| | X16834 | Mac-2 | C |
| | U18797 | H-2M3 | C |
| | U82534 | GITR | C |
| | X06143 | CD2 | C |
| | ET61114 | Ly-6C | D |
| | M18184 | Ly-6.2 | D |
| | AA097051 | Ly-6 | D |
| | D86232 | Ly-6C | D |
| | U04268 | Sca-2 | D |
| | X03151 | Thy-1 | D |
| | X66532 | Galectin-1 | D |
| Cytoskeleton | U72519 | Ena-VASP | A |
| | X54511 | Mbh-1 | C |
| | X51438 | Vimentin | D |
| Small G Protein | AA415898 | IIGP | C |
| | U05245 | TIAM-1 | C |
| | U44731 | GBP-3 | C |
| Kinase | AA110453 | a-DAG Kinase | A |
| | C81467 | JAK-2 | B |
| | L16956 | JAK-2 | B |
| | M25811 | PKC-a | C |
| | L33768 | JAK-3 | D |
| Lipid Metab. | M26270 | SCD-2 | D |
| | X70100 | MAL-1 | D |
| General Metab. | AA186047 | Glutaredoxin | C |
| | AF030343 | Ech-1 | C |
| | U59488 | p40-phox | C |
| | X61600 | b-enolase | D |
| Nucleotide Metab. | AA407018 | Thy. DNA glycos | B |
| | M37736 | Histone H2A.1 | D |
| | C81593 | Ribo. reductase | D |
| | M14223 | Ribo. reductase | D |
| | X17459 | Jk RS BP | D |
| Protein Metab. | C78483 | Elong. Fact. 1a | A |
| | AA008321 | Prot Comp. C9 | B |
| | AA215251 | GEG-154 | B |
| | X71642 | GEG-154 | B |
| | AA118121 | Isoleu. tRNA Synth. | B |
| | AA396357 | Ubiq. Conj. Enz. | C |
| | D85561 | Prot. Sub MECL-1 | C |
| | L11145 | Prot. Sub. LMP-7 | C |
| | M65270 | Cathepsin B | C |
| | U22031 | Prot Sub LMP-7 | C |
| | L11613 | Prot. Sub. LMP-2 | C |
| | X95818 | Synaptophysan | C |
| | M12302 | Granz B | D |
| | M64085 | Spi-2 | D |
| Secreted | X81582 | Ins.-Like GF BP-4 | A |
| | M17015 | Lymphotoxin | B |
| | U28493 | Lymphotactin | B |
| | U43088 | IL-17 | C |
| | L33416 | ECM-1 | C |
| | M13227 | Enkephalin | C |
| | M35590 | MIP-1b | D |
| | X12531 | MIP-1a | D |
| | X51834 | ETA-1 | D |
| Signalling | Y08361 | RIL | A |
| | X89749 | TGIF | B |
| | AF001863 | SLAP-130 | C |
| | D31943 | CIS | C |
| | AA154742 | RGS-1 | C |
| | U88327 | SOCS-2 | C |
| | AB000677 | JAB | D |
| Transcription Factor | X58636 | LEF-1 | A |
| | U43788 | OBF-1 | B |
| | AF027963 | XBP-1 | C |
| | M12848 | Myb | C |
| | M60285 | CREM | C |
| | U06924 | STAT-1 | C |
| | AA016424 | XBP-1 | C |
| | AA048098 | B-MYE | C |
| Unknown | W81812 | EST | A |
| | X61455 | L47 | A |
| | AA620163 | EST | B |
| | C77421 | EST | B |
| | L21027 | A10 | B |
| | AA061283 | EST | B |
| | AA163876 | EST | C |
| | AA289168 | EST | C |
| | AA591007 | EST | C |
| | AA726223 | EST | C |
| | AA116604 | EST | C |
| | AA138863 | EST | C |
| | W98255 | EST | C |
| | AA145371 | EST | C |
| | U13371 | EST | C |
| | U38196 | Palmityol.p55 | C |
| | W08322 | EST | C |
| | AA109907 | EST | C |
| | AA002761 | EST | D |
| | AC002393 | BAC | D |
| | C78378 | EST | D |
| | M13018 | CRIP | D |
| | AA016708 | EST | D |
| | AA117227 | EST | D |
| | W10606 | EST | D |

The genes which are known in the art to be linked to an autoimmune disorder may also serve as validation in expression studies for autoimmune disorder in conjunction with the CD25⁺ differential markers of the invention. The markers were known prior to the invention to be associated with CD25 and are provided in Table II. These markers are not to be considered as CD25⁺ differential markers of the invention. However, these markers may be conveniently used in combination with the markers of the invention (Table I) in the methods, panels, kits and compositions of the invention.

TABLE II
Common Name

TGFβ
CTLA-4
IL-10
CD-30
TNFR
IL-2
MIC-A
ICAM-1
MRL-Fas (1pr)
TNFR2
OX40
Fc R11B
Bcl-2
CD-22
PD01
SHP-1
TNF

The makers listed in Table I which are differentially expressed in CD25⁺ T cells have not been previously associated with autoimmune disorder via the CD25⁺ T cell marker.

Accordingly, the present invention pertains to the use of the markers listed in Table I, polynucleotides, and the encoded polypeptides as markers for autoimmune disorders involving CD25⁺ T cell associated disorders, and as markers of transplant rejection or acceptance. Moreover, the use of expression profiles of these genes may indicate the presence of or a risk of an autoimmune disorder. With respect to such autoimmune disorders or transplant rejections, these markers are further useful to correlate differences in levels of expression with a poor or favorable prognosis. In particular, the present invention is directed to the use of makers and panels of markers set forth in Table I or homologs thereof such as human homologs. For example, panels of the markers can be conveniently arrayed on solid supports, (i.e. biochips for use in kits). The CD25⁺ differential markers can also be useful for assessing the efficacy of a treatment or therapy of autoimmune disorders, or as a target for a treatment or therapeutic agent. The invention further provides methods for inhibiting cancer and proliferative disorders. With respect to certain embodiments relating to cancer and proliferative disorders, the invention provides methods for decreasing suppressive T cell activity or function thereby allowing for CD25⁻ T cell-mediated inhibition of cancer or proliferative disorders.

Therefore, without limitation as to mechanism, the invention is based in part on the principle that CD25⁺ T cells, and the CD25⁺ differential markers of the invention may ameliorate autoimmune disorders when expressed at levels similar or substantially similar to normal (non-diseased) cells. By activating or proliferating suppressor T cell function, certain immune responses are inhibited, and thereby inhibit or ameliorate autoimmune disorders.

Without limitation as to mechanism, the invention is also based in part on the principle that CD25⁺ T cells, and the CD25⁺ differential markers of the invention may ameliorate tissue transplant rejection by when expressed at levels similar or substantially similar to normal tissue (non-diseased e.g., without transplant rejection). By activating or proliferating suppressor T cell function, certain immune responses are inhibited, and allow for an immunosuppressive method. In certain embodiments, the CD25⁺ T cells inhibit CD8⁺ cells in a similar fashion as they inhibit CD4⁺ cells. For example, in a specific embodiment, CD4⁺CD25⁺ T cells inhibit the activation of CD8⁺ responders by inhibiting both IL-2 production and upregulation of IL-2Ra chain (CD25) expression.

Without limitation as to mechanism, the invention is also based in part on the principle that inhibiting such CD25⁺ T cells or the normal expression of CD25⁺ differential markers may ameliorate certain cancers and proliferative disorders by inhibiting suppressor T cell function and allowing for an immune response to a cancer immunogen or cancer cell.

In one aspect, the invention provides markers whose level of expression, which signifies their quantity or activity, is correlated with the presence of an autoimmune disorder. The CD25⁺ differential markers of the invention may be polynucleotides (e.g., DNA, cDNA or mRNA) or peptide(s) or polypeptides. In certain preferred embodiments, the invention is performed by detecting the presence of a transcribed polynucleotide or a portion thereof, wherein the transcribed polynucleotide comprises the marker. Alternatively, detection may be performed by detecting the presence of a protein which corresponds to the marker. The markers of the invention of Cluster Type C or Cluster Type D as set forth in Table I typically have decreased quantity or activity in autoimmune disorders as compared to normal tissue. The markers of the invention of Cluster Type A or Cluster Type B as set forth in Table I typically have increased quantity or activity in autoimmune disorders as compared to normal tissue.

In another aspect of the invention, the expression levels of the CD25⁺ differential markers are determined in a particular subject sample for which either diagnosis or prognosis information is desired. The level of expression of a number of markers simultaneously provides an expression profile, which is essentially a “fingerprint” of the activity of a marker or plurality of markers that is unique to the state of the cell. In certain embodiments, comparison of relative levels of expression is indicative of the severity of an autoimmune disorder, and as such permits for diagnostic and prognostic analysis. Moreover, by comparing relative expression profiles of an autoimmune disorder from tissue samples taken at different points in time, e.g., pre- and post-therapy and/or at different time points within a course of therapy, information regarding which genes are important in each of these stages is obtained. The identification of markers that are abnormally expressed in an autoimmune disorder versus normal tissue, as well as differentially expressed markers during severe autoimmune disorder, allows the use of this invention in a number of ways. For example, in the field of autoimmunity, comparison of expression of CD25⁺ differential marker profiles of various disease progressions states provides a method for long term prognosing, including survival. In another example mentioned above, the evaluation of a particular treatment regime may be evaluated, including whether a particular drug will act to improve the long-term prognosis in a particular patient.

The discovery of these differential expression patterns for individual or panels of CD25⁺ differential markers allows for screening of test compounds with an eye to modulating a particular expression pattern; for example, screening can be done for compounds that will convert an expression profile for a poor prognosis to a better prognosis. In certain embodiments, this may be done by making biochips comprising sets of the significant CD25⁺ differential marker genes, which can then be used in these screens. These methods can also be done on the protein level; that is protein expression levels of the autoimmune disorder-associated proteins can be evaluated for diagnostic and prognostic purposes or to screen test compounds. For example, in relation to these embodiments, significant CD25⁺ differential markers may comprise markers which are determined to have modulated activity or expression in response to a therapy regime. Alternatively, the modulation of the activity or expression of a CD25⁺ differential marker may be correlated with the diagnosis or prognosis of an autoimmune disease. In addition, the markers can be administered for gene therapy purposes, including the administration of antisense nucleic acids, or proteins (including marker polypeptides, antibodies to a marker polypeptide and other modulators of marker polypeptides) administered as therapeutic drugs.

For example, the CD25⁺ differential marker designated GITR has increased expression in CD25⁺ T cell samples, relative to control CD25⁻ T cell samples. The presence of decreased mRNA for this marker (or for other Cluster Type C or Cluster Type D markers listed in Table I, or human homologs thereof), or decreased levels of the protein products of this marker (and other Cluster Type C and D makers set forth in Table I, human homologs thereof) serve as markers for autoimmune disorders. Accordingly, modulation of Cluster Type C (such as GITR) or Cluster Type D markers to normal levels (e.g. levels similar or substantially similar to cells substantially free of an autoimmune disorder) or levels increased as compared to CD25⁻ T cells allows for amelioration of autoimmune disorders. Preferably, for the purposes of the present invention, increased levels of the markers of Cluster Type C or Cluster Type D of the invention are increased by an abnormal magnitude, wherein the level of expression is outside the standard deviation for the same marker as compared to CD25⁻ T cells. Most preferably, the Cluster Type C or Cluster Type D marker is enhanced or increased relative to CD25⁻ T cell samples by at least 2-, 3-, or 4-fold or more. Alternatively, the Cluster Type C or D marker is modulated to be similar to a control sample which is taken from a subject, tissue or cell, which is substantially free of an autoimmune disorder. One of skill in the art will appreciate the application of such control samples.

As another example, the gene designated LEF-1 has decreased expression in CD25⁺ T cell samples relative to CD25⁻ T cell samples. The presence of increased mRNA for this marker (and for other Cluster Type A and B markers set forth in Table I, or human homologs thereof), or increased levels of the protein products of this gene (and for other Cluster Type A and B makers set forth in Table I, or human homologs thereof) serve as markers for autoimmune disorders. Accordingly, modulation of Cluster Type A or Cluster Type B markers to normal levels (e.g. levels similar or substantially similar to cells substantially free of an autoimmune disorder) or levels decreased as compared to CD25⁻ T cells allows for amelioration of autoimmune disorders. Preferably for the purposes of the present invention, decreased levels of the Cluster Type A or Cluster Type B markers of the invention are decreased of abnormal magnitude, wherein the level of expression is outside the standard deviation for the same marker as compared to CD25⁺ T cells. Most preferably the marker is decreased relative to control samples by at least 2-, 3- or 4-fold or more. Alternatively, the Cluster Type A or Cluster Type B marker is modulated to be similar to a control sample which is taken from a subject, tissue or cell, which is substantially free of an autoimmune disorder. One of skill in the art will appreciate the application of such control samples.

In another embodiments of the invention, a CD25⁺ differential marker can be used as a therapeutic compound of the invention. In yet other embodiments, a modulator of a CD25⁺ differential marker of the invention may be used as a therapeutic compound of the invention, or may be used in combination with one or more other therapeutic compositions of the invention. Formulation of such compounds into pharmaceutical compositions is described in subsections below. In a specific embodiment, a protein therapeutic of the invention may comprise a soluble GITR-ligand protein. Administration of such therapeutic may induce suppressive bioactivity, and therefore may be used to ameliorate an autoimmune disorder or prevent transplant rejection. In another specific embodiment, a therapeutic of the invention may comprise a soluble version of GITR. Administration of such a therapeutic may prevent T cell suppression and therefore be used to augment cancer immunotherapy or ameliorate a cancer of the invention.

In certain specific embodiments of the invention, GITR may include isoforms or homologs of GITR including those corresponding to accession numbers XM 001593, NM 021985, AF229434, NM 005092, NM 004195, AF109216, AF241229, AF229433, AF229432, U82534, or AF125304 including polynucleotides or polypeptides of the same. Unless otherwise noted, the accession numbers provided refer to Genbank accession numbers, which can be found at http//www.ncbi.nml.nib.gov.

One of the skill in the art will recognize other controls such as by using different time points, other genes, or the presence or absence of a test compound. One of ordinary skill in the art will appreciate that other post-activation time points may be used to access expression levels of CD25⁺ and CD25⁻ T cells. For example, post-activation time points include but are not limited to 6 h, 8 h, 12 h, 15 h, 20 h, 24 h, 36 h, 48 h, 72 hours. One skilled in the art will be cognizant of the fact that a preferred detection methodology is one in which the resulting detection values are above the minimum detection limit of the methodology.

Sources of CD25⁺ Differential Markers

The polynucleotides and polypeptide markers of the invention may be isolated from any tissue or cell of a subject. In a preferred embodiment, the tissue is from blood, spleen, thymus, node or gut. In a most preferred embodiment, CD25⁺ differential markers are isolated from T cells. However, it will be apparent to one skilled in the art that tissue samples, including bodily fluids such as blood or urine, may also serve as sources from which the markers of the invention may be assessed. The tissue samples containing one or more of the markers themselves may be useful in the methods of the invention, and one skilled in the art will be cognizant of the methods by which such samples may be conveniently obtained, stored and/or preserved.

Autoimmune Disorders

The autoimmune disorders of the invention include but are not limited to Multiple Sclerosis, Insulin-Dependent Diabetes Mellitus (Type I Diabetes), Inflammatory Bowel Disease Including Ulcerative Colitis, Crohns Disease (Regional Enteritis), Systemic Lupus Erythematosis, Vasculitis, Giant cell Arteritis, Polyarteritis Nodosa, Kawasaki's Disease, Allergic Granulomatosis, Agiitis, Psoriasis, Pemphigus Vulgaris, Pemphigus Foliaceus, Bullous Pemphigoid, Cicatricial Penphigoid, Dermatitis Herpetiformis, Acute Inflammatory Demylinating Polyradiculoneuropathy (Guillain-Barre Syndrome), Chronic Inflammatory Demyleinating Polyradiculoneuropathy, Peripheral Nerve Vasculitis, Lambert-Eaton Myasthenic Syndrome, Transverse Myelitis, Optic Neuritis, Neuromyelitis Optica, Autoimmune Gastritis, Hypophysitis, Polyglandular Autoimmune Endocrine Disease, Autoimmune Thyroiditis (Graves Disease, Hashimotos Thyroiditis), Autoimmune Disease of the Adrenal, Hypoparathyroidism, Insulin Autoimmune Syndrome, Autoimmune Uveitis, Episcleritis, Scleritis, Sjorgrens Syndrome, Behcets Syndrome, Retinal Vasculitis, Myasthenia Gravis, Idiopathic Inflammatory Myopathy, Polymyositis, Dermatomyositis, Autoimmune Myocardits, Dilated Cardiomyopathy, Autoimmune Diseases of the Reproductive Glands including Oophoritis Orchitis, Premature Ovarian Failure, Aplastic Anemia, Myelodysplastic Syndromes, Paroxysmal Nocturnal Hemoglobinuria, Red Cell Aplasia, Chronic Neutropenia, Autoimmune Thrombocytopenia, Autoimmune Hemolytic Anemia, Antiphospholipid Antibody Syndromes, Pernicious Anemia, Spontaneous Acquired Inhibitors of Coagulant Factors, Autoimmune Hepatitis, Primary Biliary Cirrhosis, Hepatitis C Associated Autoimmunity, Wegeners Granulomatosis, Sarcoidosis, Scleroderma, Asthma, Allergic Rhinitis, Metal Allergy, Contact Hypersensitivity, Drug Induced Autoimmunity, Immunoglobulin a Nephropathy, Membranous Nephropathy, Idiopathic Nephritic Syndrome, Mesangiocapillary Glomerulonephritis, Poststreptococcal Glomerulonephritis, Tubulointerstitial Nephritis, Goodpastures Syndrome, and Interstitial Cystitis. The compositions and methods of the invention are particularly useful in relation to rheumatoid arthritis; systemic lupus erythematosis; psoriasis; multiple sclerosis; insulin-dependent diabetes mellitus (type I diabetes); inflammatory bowel disease including ulcerative colitis and Crohn's disease (regional enteritis); asthma; or allertic rhinitis. The compositions and methods of the invention are most useful in relation to rheumatoid arthritis, multiple sclerosis or insulin-dependent diabetes mellitus (type I diabetes).

The cancers and proliferative disorder of the invention include but are not limited to renal cancer, melanoma, breast cancer, lymphoma, or multiple myeloma. The compositions and methods of the invention are particularly useful in relation to renal cancer or melanoma.

Transplant rejection includes immune responses following transplantation of any organ or tissue (including but not limited to kidney, heart, skin, liver, pancreas, small bowel, or lung).

Isolated Polynucleotides

One aspect of the invention pertains to isolated polynucleotide molecules comprising CD25⁺ differential markers (e.g., mRNA) of the invention, or polynucleotides which encode polypeptide CD25⁺ differential markers of the invention, or fragments thereof. Another aspect of the invention pertains to isolated polynucleotide fragments sufficient for use as hybridization probes to identify the polynucleotide molecules encoding the markers for the invention in a sample, as well as nucleotide fragments for use as PCR primers of the amplification or mutation of the nucleic acid molecules which encode the CD25⁺ differential markers of the invention.

A polynucleotide molecule of the present invention, e.g., a polynucleotide molecule having the nucleotide sequence of one of the CD25⁺ differential markers listed in Table I, or homolog thereof, or a portion thereof, can be isolated using standard molecular biology techniques and the sequence information provided herein as well as sequence information known in the art. Using all or portion of the polynucleotide sequence of one of the CD25⁺ differential markers listed Table I (or a homolog thereof) as a hybridization probe, a CD25⁺ differential marker gene of the invention or a polynucleotide molecule encoding a CD25⁺ differential marker polypeptide of the invention can be isolated using standard hybridization and cloning techniques (e.g. as described in Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold spring Harbor, N.Y., 1989).

A polynucleotide of the invention can be amplified using cDNA, mRNA or alternatively, genomic DNA, as a template and appropriate oligonucleotide primers according to standard PCR amplification techniques. The polynucleotide so amplified can be cloned into an appropriate vector and characterized by DNA sequence analysis. Furthermore, oligonucleotides corresponding to CD25⁺ differential marker nucleotide sequences, or nucleotide sequences encoding a marker of the invention can be prepared by standard synthetic techniques, e.g., using an automated DNA synthesizer.

In another preferred embodiment, an isolated polynucleotide molecule of the invention comprises a polynucleotide molecule which is a complement of the nucleotide sequence of a CD25⁺ differential marker of the invention (e.g., a marker listed in Table I, or homolog thereof), or a portion of any of these nucleotide sequences. A polynucleotide molecule which is complementary to such a nucleotide sequence is one which is sufficiently complementary to the nucleotide sequence such that it can hybridize to the nucleotide sequence, thereby forming a stable duplex.

The polynucleotide molecule of the invention, moreover, can comprise only a portion of the polynucleotide sequence of a CD25⁺ differential marker polynucleotide of the invention, or a gene encoding a polypeptide of the invention, for example, a fragment which can be used as a probe or primer. The probe/primer typically comprises substantially purified oligonucleotide. The oligonucleotide typically comprises a region of nucleotide sequence that hybridizes under stringent conditions to at least about 7 or 15, preferably about 20 or 25, more preferably about 50, 75, 100, 125, 150, 175, 200, 225, 250, 275, 300, 325, 350, 400 or more consecutive nucleotides of a CD25⁺ differential marker polynucleotide, or a polynucleotide encoding a CD25⁺ differential marker polypeptide of the invention.

Probes based on the nucleotide sequence of a CD25⁺ differential marker gene or of a polynucleotide molecule encoding a marker polypeptide of the invention can be used to detect transcripts or genomic sequences corresponding to the marker gene(s) and/or marker polypeptide(s) of the invention. In preferred embodiments, the probe comprises a label group attached thereto, e.g., the label group can be a radioisotope, a fluorescent compound, an enzyme, or an enzyme co-factor. Such probes can be used as a part of a diagnostic test kit for identifying cells or tissue which misexpress (e.g., over- or under-express) a marker polynucleotide or polypeptide of the invention, or which have greater or fewer copies of a marker gene of the invention. For example, a level of a marker in a sample of cells from a subject may be detected, the amount of polypeptide or mRNA transcript of a gene encoding a marker polypeptide may be determined, or the presence of mutations or deletions of a marker gene of the invention may be assessed.

The invention further encompasses polynucleotide molecules that differ from the polynucleotide sequences of the markers listed in Table I, due to degeneracy of the genetic code and which thus encode the same proteins as those encoded by the genes shown in Table I.

The invention also specifically encompasses homologs of the markers listed in Table I of other species, particularly human homology of the markers listed in Table I. Gene homologs are well understood in the art and are available using databases or search engines such as the Pubmed-Entrez database available at <http:www.ncbi.nlm.nihigov/query.fcgi>.

The invention also encompasses polynucleotide molecules which are structurally different from the molecules described above (i.e. which have a slight altered sequence), but which have substantially the same properties as the molecules above (e.g., encoded amino acid sequences, or which are changed only in nonessential amino acid residues). Such molecules include allelic variants, and are described in greater detail in subsections herein.

In addition to the nucleotide sequences of the markers listed in Table I, it will be appreciated by those skilled in the art that DNA sequence polymorphisms that lead to changes in the amino acid sequences of the proteins encoded by the markers listed in Table I may exist within a population (e.g., the human population). Such genetic polymorphism in the markers listed in Table I may exist among individuals within a population due to natural allelic variation. An allele is one of a group of genes which occur alternatively at a given genetic locus. In addition it will be appreciated that DNA polymorphisms that affect RNA expression levels can also exist that may affect the overall expression level of that gene (e.g., by affecting regulation or degradation). As used herein, the phrase “allelic variant” includes a nucleotide sequence which occurs at a given locus or to a polypeptide encoded by the nucleotide sequence.

Polynucleotide molecules corresponding to natural allelic variants and homologues of the marker genes, or genes encoding the marker proteins of the invention can be isolated based on their homology to the markers listed in Table I, using the cDNAs disclosed herein, or a portion thereof, as a hybridization probe according to standard hybridization techniques under stringent hybridization conditions. Polynucleotide molecules corresponding to natural allelic variants and homologues of the marker genes of the invention can further be isolated by mapping to the same chromosome or locus as the marker genes or genes encoding the marker proteins of the invention.

In another embodiment, an isolated polynucleotide molecule of the invention is at least 15, 20, 25, 30, 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000 or more nucleotides in length and hybridizes under stringent conditions to a polynucleotide molecule corresponding to a nucleotide sequence of a marker gene or gene encoding a marker protein of the invention. In certain embodiments, the hybridization under stringent conditions is intended to describe conditions for hybridization and washing under which nucleotide sequences at least 60% homologous to each other typically remain hybridized to each other. Preferably, the conditions are such that sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. Preferably, an isolated polynucleotide molecule of the invention that hybridizes under stringent conditions to the sequence of one of the markers set forth in Table I corresponds to a naturally-occurring polynucleotide molecule.

In addition to naturally-occurring allelic variants of the marker gene and gene encoding a marker protein of the invention sequences that may exist in the population, the skilled artisan will further appreciate that changes can be introduced by mutation into the nucleotide sequences of the marker genes or genes encoding the marker proteins of the invention, thereby leading to changes in the amino acid sequence of the encoded proteins, without altering the functional activity of these proteins. For example, nucleotide substitutions leading to amino acid substitutions at “non-essential” amino acid residues can be made. A “non-essential” amino acid residue is a residue that can be altered from the wild-type sequence of a protein without altering the biological activity, whereas an “essential” amino acid residue is required for biological activity. For example, amino acid residues that are conserved among allelic variants or homologs of a gene (e.g., among homologs of a gene from different species) are predicted to be particularly unamenable to alteration.

Accordingly, another aspect of the invention pertains to polynucleotide molecules encoding a marker protein of the invention that contain changes in amino acid residues that are not essential for activity. Such proteins differ in amino acid sequence from the marker proteins encoded by the markers listed in Table I, yet retain biological activity. In one embodiment, the protein comprises an amino acid sequence at least about 60%, 65%, 70%, 75%, 80%, 85%, 90%, 95%, 98% or more homologous to a marker protein of the invention.

In yet other aspects of the invention, polynucleotides of a CD25⁺ differential marker may comprise one or more mutations. An isolated polynucleotide molecule encoding a protein with a mutation in a CD25⁺ differential marker protein of the invention can be created by introducing one or more nucleotide substitutions, additions or deletions into the nucleotide sequence of the gene encoding the marker protein, such that one or more amino acid substitutions, additions or deletions are introduced into the encoded protein. Such techniques are well known in the art. Mutations can be introduced into the CD25⁺ differential marker polynucleotides of the invention (e.g., a marker listed in Table I) by standard techniques, such as site-directed mutagenesis and PCR-mediated mutagenesis. Preferably, conservative amino-acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of a coding sequence of a CD25⁺ differential gene of the invention, such as by saturation mutagenesis, and the resultant mutants can be screened for biological activity to identify mutants that retain activity. Following mutagenesis, the encoded protein can be expressed recombinantly and the activity of the protein can be determined.

Another aspect of the invention pertains to isolated polynucleotide molecules which are antisense to the CD25⁺ differential marker genes and genes encoding CD25⁺ differential marker proteins of the invention. An “antisense” polynucleotide comprises a nucleotide sequence which is complementary to a “sense” polynucleotide encoding a protein, (e.g., complementary to the coding strand of a double-stranded cDNA molecule or complementary to an mRNA sequence). Accordingly, an antisense polynucleotide can hydrogen bond to a sense polynucleotide. The antisense polynucleotide can be complementary to an entire coding strand of a gene of the invention or to only a portion thereof. In one embodiment, an antisense polynucleotide molecule is antisense to a “coding region” of the coding strand of a nucleotide sequence of the invention. The term “coding region” includes the region of the nucleotide sequence comprising codons which are translated into amino acid. In another embodiment, the antisense polynucleotide molecule is antisense to a “noncoding region” of the coding strand of a nucleotide sequence of the invention.

Antisense polynucleotides of the invention can be designed according to the rules of Watson and Crick base pairing. The antisense polynucleotide molecule can be complementary to the entire coding region of an mRNA corresponding to a gene of the invention, but more preferably is an oligonucleotide which is antisense to only a portion of the coding or noncoding region. An antisense oligonucleotide can be, for example, about 5, 10, 15, 20, 25, 30, 35, 40, 45 or 50 nucleotides in length. An antisense polynucleotide of the invention can be constructed using chemical synthesis and enzymatic ligation reactions using procedures known in the art. For example, an antisense polynucleotide (e.g., an antisense oligonucleotide) can be chemically synthesized using naturally occurring nucleotides or variously modified nucleotides designed to increase the biological stability of the molecules or to increase the physical stability of the duplex formed between the antisense and sense polynucleotides, (e.g., phosphorothioate derivatives and acridine substituted nucleotides) can be used. Examples of modified nucleotides which can be used to generate the antisense polynucleotide include 5-fluorouracil, 5-bromouracil, 5-chlorouracil, 5-iodouracil, hypoxanthine, xantine, 4-acetylcytosine, 5-(carboxyhydroxyhnethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladen4exine, unacil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense polynucleotide can be produced biologically using an expression vector into which a polynucleotide has been subcloned in an antisense orientation (i.e., RNA transcribed from the inserted polynucleotide will be of an antisense orientation to a target polynucleotide of interest, described further in the following subsection).

The antisense polynucleotide molecules of the invention are typically administered to a subject or generated in situ such that they hybridize with or bind to cellular mRNA and/or genomic DNA encoding a marker protein of the invention to thereby inhibit expression of the protein, e.g., by inhibiting transcription and/or translation. The hybridization can be by conventional nucleotide complementarity to form a stable duplex, or, for example, in the cases of an antisense polynucleotide molecule which binds to DNA duplexes, through specific interactions in the major groove of the double helix. An example of a route of administration of antisense polynucleotide molecules of the invention include direct injection at a tissue site (e.g., lymph node or blood). Alternatively, antisense polynucleotide molecules can be modified to target selected cells and then administered systemically. For example, for systemic administration, antisense molecules can be modified such that they specifically bind to receptors or antigens expressed on a selected cell surface, e.g., by linking the antisense polynucleotide molecules to peptides or antibodies which bind to cell surface receptors or antigens. For example, one method to target CD25⁺ T cells is to use GITR. One method to target CD25− T cells is to use ITM2. The antisense polynucleotide molecules can also be delivered to cells using the vectors described herein. To achieve sufficient intracellular concentrations of the antisense molecules, vector constructs in which the antisense polynucleotide molecule is placed under the control of a strong pol II or pol III promoter are preferred.

In yet another embodiment, the antisense polynucleotide molecule of the invention is an α-anomeric polynucleotide molecule. An α-anomeric polynucleotide molecule forms specific double-stranded hybrids with complementary RNA in which, contrary to the usual β-units, the strands run parallel to each other (Gaultier et al. (1987) Polynucleotides. Res. 15:6625-6641). The antisense polynucleotide molecule can also comprise a 2′-o-methylribonucleotide (Inoue et al. (1987) Polynucleotides Res. 15:6131-6148) or a chimeric RNA-DNA analogue (Inoue et al. (1987) FEBS Lett. 215:327-330).

In still another embodiment, an antisense polynucleotide of the invention is a ribozyme. Ribozymes are catalytic RNA molecules with ribonuclease activity which are capable of cleaving a single-stranded polynucleotide, such as an mRNA, to which they have a complementary region. Thus, ribozymes (e.g., hammerhead ribozymes (described in Haselhoif and Gerlach (1988) Nature 334:585-591)) can be used to catalytically cleave mRNA transcripts of the CD25⁺ differential marker genes of the invention (e.g., as set forth in Table I) to thereby inhibit translation of said mRNA. A ribozyme having specificity for a marker protein-encoding polynucleotide can be designed based upon the nucleotide sequence of a gene of the invention, disclosed herein. For example, a derivative of a Tetrahymena L-19 IVS RNA can be constructed in which the nucleotide sequence of the active site is complementary to the nucleotide sequence to be cleaved in a marker protein-encoding mRNA. See, e.g., Cech et al. U.S. Pat. No. 4,987,071; and Cech et al. U.S. Pat. No. 5,116,742. Alternatively, mRNA transcribed from a gene of the invention can be used to select a catalytic RNA having a specific ribonuclease activity from a pool of RNA molecules. See, e.g., Bartel, D. and Szostak, J. W. (1993) Science 261:1411-1418.

Alternatively, expression of a CD25⁺ differential marker gene of the invention can be inhibited by targeting nucleotide sequences complementary to the regulatory region of these genes (e.g., the promoter and/or enhancers) to form triple helical structures that prevent transcription of the gene in target cells. See generally, Helene, C. (1991) Anticancer Drug Des. 6(6):569-84; Helene, C. et al. (1992) Ann. N. Y. Acad Sci. 660:27-36; and Maher, L. J. (1992) Bioassays 14(12):807-15.

Expression of the marker genes, and genes encoding marker proteins of the invention, can also be inhibited using RNA interference (“RNAᵢ”). This is a technique for post transcriptional gene silencing (“PTGS”), in which target gene activity is specifically abolished with cognate double-stranded RNA (“dsRNA”). RNAᵢ resembles in many aspects PTGS in plants and has been detected in many invertebrates including trypanosome, hydra, planaria, nematode and fruit fly (Drosophila melanogaster). It may be involved in the modulation of transposable element mobilization and antiviral state formation. RNA, in mammalian systems is disclosed in PCT application WO 00/63364 which is incorporated by reference herein in its entirety. Basically, dsRNA of at least about 21 nucleotides, homologous to the target marker is introduced into the cell and a sequence specific reduction in gene activity is observed. See e.g., Elbashir S M et al. Duplexes of 21-nucleotide RNAs mediate RNA interference in cultured mammalian cells, Nature May 24;411(6836):494-8 (2001).

In yet another embodiment, the polynucleotide molecules of the present invention can be modified at the base moiety, sugar moiety or phosphate backbone to improve, e.g., the stability, hybridization, or solubility of the molecule. For example, the deoxyribose phosphate backbone of the polynucleotide molecules can be modified to generate peptide polynucleotides (see Hyrup B. et al. (1996) Bioorganic & Medicinal Chemistry 4(1): 523). As used herein, the terms “peptide polynucleotides” or “PNAs” refer to polynucleotide mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs can be used in therapeutic and diagnostic applications. For example, PNAs can be used as antisense or antigene agents for sequence-specific modulation of marker gene expression by, for example, inducing transcription or translation arrest or inhibiting replication. PNAs of the polynucleotide molecules of the invention (e.g., set forth in Table I or homologs thereof) can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of the polynucleotide molecules of the invention can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Polynucleotides Res. 24 (17): 3357-63. For example, a DNA chain can be synthesized on a solid support using standard phosphoramidite coupling chemistry and modified nucleoside analogs, (e.g., 5′-(4-methoxytrityl)amino-5′-deoxy-thymidine phosphoramidite), can be used as a spacer between the PNA and the 5′ end of DNA (Mag, M. et al. (1989) Polynucleotide Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med Chem. Lett. 5: 1119-11124).

In other embodiments, the oligonucleotide may include other appended groups such as peptides (e.g., for targeting host cell receptors in vivo), or agents facilitating transport across the cell membrane (See, e.g., Letsinger et al. (1989) Proc. Natl. Acad. Sci. USA 86:6553-6556; Lemaitre et al. (1987) Pros. Natl. Acad Sci. USA 84:648-652; PCT Publication No. W088/09810) or the blood-kidney barrier (See, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent). Finally, the oligonucleotide may be detectably labeled, either such that the label is detected by the addition of another reagent (e.g., a substrate for an enzymatic label), or is detectable immediately upon hybridization of the nucleotide (e.g., a radioactive label or a fluorescent label (e.g., a molecular beacon, as described in U.S. Pat. No. 5,876,930).

Isolated Polypeptides

Several aspects of the invention pertain to isolated CD25⁺ differential marker proteins, and biologically active portions thereof, as well as polypeptide fragments suitable for use as immunogens to raise anti-marker protein antibodies. In one embodiment, native marker proteins can be isolated from cells or tissue sources by an appropriate purification scheme using standard protein purification techniques. In another embodiment, marker proteins are produced by recombinant DNA techniques. Alternative to recombinant expression, a marker protein or polypeptide can be synthesized chemically using standard peptide synthesis techniques.

The invention provides marker proteins encoded by a CD25⁺ differential marker gene set forth in Table I, or homologs thereof, including human homologs. In other embodiments, the marker protein is substantially homologous to a marker protein encoded by a marker listed in Table I, and retains the functional activity of the marker protein, yet differs in amino acid sequence due to natural allelic variation or mutagenesis, as described in detail above. Accordingly, in another embodiment, the CD25⁺ differential marker protein is a protein which comprises an amino acid sequence at least about 60%, 65%, 70%, 75%, 80%, 85%, 90%, 95%, 98% or more homologous to the amino acid sequence encoded by a marker listed in Table I.

To determine the percent identity of two amino acid sequences or of two polynucleotide sequences, the sequences are aligned for optimal comparison purposes (e.g., gaps can be introduced in one or both of a first and a second amino acid or polynucleotide sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or polynucleotide “identity” is equivalent to amino acid or polynucleotide “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM 120 weight residue table, a gap length penalty of 12 and a gap penalty of 4.

The polynucleotide and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to polynucleotide molecules of the invention. BLAST protein searches can be performed with the XBLAST program, score=50, wordlength=3 to obtain amino acid sequences homologous to marker protein molecules of the invention. To obtain gapped alignments for comparison purposes, Gapped BLAST can be utilized as described in Altschul et al., (1997) Polynucleotides Res. 25(17):3389-3402. When utilizing BLAST and Gapped BLAST programs, the default parameters of the respective programs (e.g., XBLAST and NBLAST) can be used. See <http://www.ncbi.nim.nih.gov.>

The invention also provides chimeric or fusion marker proteins. Within a marker fusion protein the polypeptide can correspond to all or a portion of a marker protein. In a preferred embodiment, a marker fusion protein comprises at least one biologically active portion of a marker protein. Within the fusion protein, the term “operatively linked” is intended to indicate that the marker polypeptide and the non-marker polypeptide are fused in-frame to each other. The non-marker polypeptide can be fused to the N-terminus or C-terminus of the marker polypeptide.

For example, in one embodiment, the fusion protein is a GST-marker fusion protein in which the marker sequences are fused to the C-terminus of the GST sequences. Such fusion proteins can facilitate the purification of recombinant marker proteins.

In another embodiment, the fusion protein is a marker protein containing a heterologous signal sequence at its N-terminus. In certain host cells (e.g., mammalian host cells), expression and/or secretion of marker proteins can be increased through use of a heterologous signal sequence. Such signal sequences are well known in the art.

The marker fusion proteins of the invention can be incorporated into pharmaceutical compositions and administered to a subject in vivo, as described herein. The marker fusion proteins can be used to affect the bioavailability of a marker protein substrate. Use of marker fusion proteins may be useful therapeutically for the treatment of or prevention of damage (e.g., organ damage resulting from reperfusion) caused by, for example, (i) aberrant modification or mutation of a gene encoding a marker protein; (ii) mis-regulation of the marker protein-encoding gene; and (iii) aberrant post-translational modification of a marker protein.

Moreover, the marker-fusion proteins of the invention can be used as immunogens to produce anti-marker protein antibodies in a subject, to purify marker protein ligands and in screening assays to identify molecules which inhibit the interaction of a marker protein with a marker protein substrate.

Preferably, a marker chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols In Molecular Biology, eds. Ausubel et al. John Wiley & Sons: 1992). Moreover, many expression vectors are commercially available that already encode a fusion moiety (e.g., a GST polypeptide). A marker protein-encoding polynucleotide can be cloned into such an expression vector such that the fusion moiety is linked in-frame to the marker protein.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a polynucleotide sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods.

Alternatively, the signal sequence can be linked to the protein of interest using a sequence which facilitates purification, such as with a GST domain.

The present invention also pertains to variants of the CD25⁺ differential marker proteins of the invention which function as either agonists or as antagonists to the marker proteins. In several embodiments of the invention antagonists or agonists of the CD25⁺ differential markers of the invention are therapeutic agents of the invention. For example, agonists of a Cluster Type C or Cluster Type D CD25⁺ differential marker can increase the activity or expression of such a marker and therefore ameliorate an autoimmune disorder in a subject wherein said markers are abnormally decreased in level or activity. In one embodiment, the CD25⁺ differential marker GITR is abnormally decreased in activity or expression levels in a subject diagnosed with or suspected of having an autoimmune disorder. In this embodiment, treatment of such a subject may comprise administering an agonist of GITR wherein such agonist provides increased activity or expression of GITR.

In another embodiment of the invention, the CD25⁺ differential marker GITR is abnormally increased in activity or expression levels in a subject diagnosed with or suspected of having cancer or a proliferative disorder, or a decreased expression of normal levels of GITR is desired. In this embodiment, treatment of such a subject may comprise administering an antagonist of GITR wherein such antagonist provides decreased activity or expression of GITR.

In other embodiments of the invention an agonist or antagonist of a CD25⁺ differential marker is a variant of a marker of the invention. Variants of the marker proteins can be generated by mutagenesis, e.g., discrete point mutation or truncation of a marker protein.

In certain embodiments, an agonist of the marker proteins can retain substantially the same, or a subset, of the biological activities of the naturally occurring form of a marker protein or may enhance an activity of a marker protein. In certain embodiments, an antagonist of a marker protein can inhibit one or more of the activities of the naturally occurring form of the marker protein by, for example, competitively modulating an activity of a marker protein. Thus, specific biological effects can be elicited by treatment with a variant of limited function. In one embodiment, treatment of a subject with a variant having a subset of the biological activities of the naturally occurring forth of the protein has fewer side effects in a subject relative to treatment with the naturally occurring form of the marker protein.

Variants of a marker protein which function as either marker protein agonists or as marker protein antagonists can be identified by screening combinatorial libraries of mutants, e.g., truncation mutants, of a marker protein for marker protein agonist or antagonist activity. In one embodiment, a variegated library of CD25⁺ differential marker protein variants is generated by combinatorial mutagenesis at the polynucleotide level and is encoded by a variegated gene library. In certain embodiments, such protein may be used for example as a therapeutic protein of the invention. A variegated library of marker protein variants can be produced by, for example, enzymatically ligating a mixture of synthetic oligonucleotides into gene sequences such that a degenerate set of potential marker protein sequences is expressible as individual polypeptides, or alternatively, as a set of larger fusion proteins (e.g., for phage display) containing the set of marker protein sequences therein. There are a variety of methods which can be used to produce libraries of potential marker protein variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential marker protein sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1055; Ike et al. (1983) Polynucleotide Res. 11:477).

Methods and compositions for screening for protein inhibitors or activators are known in the art (see U.S. Pat. Nos. 4,980,281, 5,266,464, 5,688,635, and 5,877,007, which are incorporated herein by reference).

In addition, libraries of fragments of a protein coding sequence corresponding to a CD25⁺ differential marker protein of the invention can be used to generate a variegated population of marker protein fragments for screening and subsequent selection of variants of a marker protein. In one embodiment, a library of coding sequence fragments can be generated by treating a double stranded PCR fragment of a marker protein coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the marker protein.

Several techniques are known in the art for screening gene products of combinatorial libraries made by point mutations or truncation, and for screening cDNA libraries for gene products having a selected property. The most widely used techniques, which are amenable to high-throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify marker variants (Arkin and Yourvan (1992) Proc. Natl. Acad. Sci. USA 89:7811-7815; Delgrave et al. (1993) Protein Engineering 6(3):327-33 1).

Antibodies

In another aspect, the invention includes antibodies that are specific to proteins corresponding to CD25⁺ differential markers of the invention. Preferably the antibodies are monoclonal, and most preferably, the antibodies are humanized, as per the description of antibodies described below.

In another aspect, the invention provides methods of making an isolated hybridoma which produces an antibody useful for diagnosing a patient or animal with an autoimmune disorder. In this method, a protein corresponding to a CD25⁺ differential marker of the invention is isolated (e.g., by purification from a cell in which it is expressed or by transcription and translation of a polynucleotide encoding the protein in vivo or in vitro using known methods). A vertebrate, preferably a mammal such as a mouse, rabbit or sheep, is immunized using the isolated protein or protein fragment. The vertebrate may optionally (and preferably) be immunized at least one additional time with the isolated protein or protein fragment, so that the vertebrate exhibits a robust immune response to the protein or protein fragment. Splenocytes are isolated from the immunized vertebrate and fused with an immortalized cell line to form hybridomas, using any of a variety of methods well known in the art. Hybridomas formed in this manner are then screened using standard methods to identify one or more hybridomas which produce an antibody which specifically binds with the protein or protein fragment. The invention also includes hybridomas made by this method and antibodies made using such hybridomas.

An isolated marker protein, or a portion or fragment thereof, can be used as an immunogen to generate antibodies that bind CD25⁺ differential marker proteins using standard techniques for polyclonal and monoclonal antibody preparation. A full-length marker protein can be used or, alternatively, the invention provides antigenic peptide fragments of these proteins for use as immunogens. The antigenic peptide of a CD25⁺ differential marker protein comprises at least 8 amino acid residues of an amino acid sequence encoded by a marker set forth in Table I, and encompasses an epitope of a marker protein such that an antibody raised against the peptide forms a specific immune complex with the marker protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of the marker protein that are located on the surface of the protein, (e.g., hydrophilic regions), as well as regions with high antigenicity.

A marker protein immunogen typically is used to prepare antibodies by immunizing a suitable subject, (e.g., rabbit, goat, mouse or other mammal) with the immunogen. An appropriate immunogenic preparation can contain, for example, recombinantly expressed marker protein or a chemically synthesized marker polypeptide. The preparation can further include an adjuvant, such as Freund's complete or incomplete adjuvant, or similar immunostimulatory agent. Immunization of a suitable subject with an immunogenic marker protein preparation induces a polyclonal anti-marker protein antibody response. Techniques for preparing, isolating and using antibodies are well known in the art. (See generally D. Lane and E. Harlow in Antibodies: A laboratory Manual, Cold Spring Harbor Laboratory Press, New York (1990)).

Accordingly, another aspect of the invention pertains to monoclonal or polyclonal anti-marker protein antibodies. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to marker proteins. The term “monoclonal antibody” or “monoclonal antibody composition”, as used herein, includes a population of antibody molecules that contain only one species of an antigen binding site capable of immunoreacting with a particular epitope. A monoclonal antibody composition thus typically displays a single binding affinity for a particular marker protein with which it immunoreacts.

Polyclonal anti-marker protein antibodies can be prepared as described above by immunizing a suitable subject with a marker protein of the invention. The anti-marker protein antibody titer in the immunized subject can be monitored over time by standard techniques, such as with an enzyme linked immunosorbent assay (ELISA) using immobilized marker protein. If desired, the antibody molecules directed against marker proteins can be isolated from the mammal (e.g., from the blood) and further purified by well known techniques, such as protein A chromatography, to obtain the IgG fraction. At an appropriate time after immunization, e.g., when the anti-marker protein antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad, Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a mycloma) is fused to lymphocytes (typically splenocytes) from a mammal immunized with a marker protein immunogen as described above, and the culture supernatants of the resulting hybridoma cells are screened to identify a hybridoma producing a monoclonal antibody that binds to a marker protein of the invention.

Any of the many well known protocols used for fusing lymphocytes and immortalized cell lines can be applied for the purpose of generating an anti-marker protein monoclonal antibody (see, e.g., G. Galfre et al. (1977) Nature 266:SSOS2; Gefter et al. Somatic Cell Genet., cited supra; Letter, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, axninopterin and thymidine (“HAT medium”). Any of a number of myeloma cell lines can be used as a fusion partner according to standard techniques, e.g., the P3-NS1/1-Ag4-1, P3-x63-Ag8.653 or Sp210-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind to a marker protein, e.g., using a standard ELISA assay.

Alternative to preparing monoclonal antibody-secreting hybridomas, a monoclonal anti-marker protein antibody can be identified and isolated by screening a recombinant combinatorial immunoglobulin library (e.g., an antibody phase display library) with marker protein to thereby isolate immunoglobulin library members that bind to a marker protein. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Fuchs et al. (1991) Bio/Technology 9:1370-1372; Hay et al. (1992) Hum. Antibod Hybridomas 3:81-85; Huse et al. (1989) Science 246:1275-1281; Griffiths et al. (1993) EMBO J 12:725-734; and McCafferty et al. Nature (1990) 348:552-554.

Additionally, recombinant anti-marker protein antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Cabilly et al. U.S. Pat. No. 4,816,567; Better et al. (1988) Science 240:1041-1043; Liu et al. (1987) Proc. Natl. Acad Sci. USA 84:3439-3443; Liu et al. (1987) J. Immunol. 139:3521 3526;Verhoeyan et al. (1988) Science 239:1534; and Beidler et al. (1988) J. Immunol. 141:4053-4060.

Humanized antibodies are particularly desirable for therapeutic treatment of human subjects. Humanized forms of non-human (e.g. murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues forming a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the constant regions being those of a human immunoglobulin consensus sequence. The humanized antibody will preferably also comprise at least a portion of an immunoglobulin constant region (Fc), typically that of a human immunoglobulin (Jones et al. Nature 321: 522-525 (1986); Riechmann et al, Nature 323: 323-329 (1988); and Presta Curr.Op.Struct.Biol. 2: 594-596 (1992).

Such humanized antibodies can be produced using transgenic mice which are incapable of expressing endogenous immunoglobulin heavy and light chain genes, but which can express human heavy and light chain genes. The transgenic mice are immunized in the normal fashion with a selected antigen, (e.g., all or a portion of a polypeptide corresponding to a marker of the invention). Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing humanized antibodies, see Lonberg and Huszar (1995) Int. Rev. Immunol. 13:65-93. For a detailed discussion of this technology for producing humanized antibodies and humanized monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide humanized antibodies directed against a selected antigen using technology similar to that described above.

Humanized antibodies which recognize a selected epitope can be generated using a technique referred to as “guided selection.” In this approach a selected non-human monoclonal antibody, e.g., a murine antibody, is used to guide the selection of a humanized antibody recognizing the same epitope (Jespers et al., 1994, Bio/technology 12:899-903).

Commercially available anti-marker antibodies may also be used in the methods of the invention. For example the anti-GITR/TNFRSF18# AF524 commercially available from R&D Systems (Minneapolis, Minn.) may be used to detect GITR protein.

An anti-marker protein antibody can be used to isolate a marker protein of the invention by standard techniques, such as affinity chromatography or immunoprecipitation. An anti-marker protein antibody can facilitate the purification of natural marker proteins from cells and of recombinantly produced marker proteins expressed in host cells. Moreover, an anti-marker protein antibody can be used to detect a CD25⁺ differential marker protein (e.g., in a cellular lysate or cell supernatant on the cell surface) in order to evaluate the abundance and pattern of expression of the marker protein. Anti-marker protein antibodies can be used diagnostically to monitor protein levels in tissue as part of a clinical testing procedure, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatasc, galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Anti-marker antibodies of the invention are also useful for targeting a therapeutic to a cell or tissue comprising the antigen of the anti-marker antibody. For example, a therapeutic such as a small molecule, a cytotoxic agent (in the case of treatment of cancer), or other therapeutic of the invention can be linked to the anti-marker antibody in order to target the therapeutic to the cell or tissue comprising the marker antigen. Such method is particularly useful in connection with CD25⁺ differential markers which are surface markers.

In a specific embodiment, antibodies to a CD25⁺ differential marker may be used to eliminate this population in vivo by activating the complement system or mediating ADCC, or cause uptake of the antibody coated cells by the RE system. In one example of this embodiment, an anti-GITR antibody is used.

Recombinant Expression Vectors and Host Cells

Another aspect of the invention pertains to vectors, preferably expression vectors, containing a polynucleotide encoding a CD25⁺ differential marker protein of the invention (or a portion thereof). As used herein, the term “vector” includes a polynucleotide molecule capable of transporting another polynucleotide to which it has been linked. One type of vector is a “plasmid”, which includes a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors host cell (e.g., replication defective retroviruses, adenoviruses and adeno-associated viruses), which serve equivalent functions.

The recombinant expression vectors of the invention comprise a polynucleotide of the invention in a form suitable for expression of the polynucleotide in a host cell, which means that the recombinant expression vectors include one or more regulatory sequences, selected on the basis of the host cells to be used for expression, which is operatively linked to the polynucleotide sequence to be expressed. Within a recombinant expression vector, “operably linked” is intended to mean that the nucleotide sequence of interest is linked to the regulatory sequences) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by polynucleotides as described herein (e.g., marker proteins, mutant forms of marker proteins, fusion proteins, and the like).

The recombinant expression vectors of the invention can be designed for expression of marker proteins in prokaryotic or eukaryotic cells. For example, marker proteins can be expressed in bacterial cells such as E. coli, insect cells (using baculovirus expression vectors) yeast cells or mammalian cells. In certain embodiments, such protein may be used, for example, as a therapeutic protein of the invention. Suitable host cells are discussed further in Goeddel, Gene Expression Technology. Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D, B. and Johnson, K. S. (1988) Gene 67:31-40), pMAL (New England Biolabs, Beverly, Mass.) and pRITS (Pharmacia, Piscataway, N.J.) which fuse glutathione S transferase (GST), maltose E binding protein, or protein A, respectively, to the target recombinant protein.

Purified fusion proteins can be utilized in marker activity assays, (e.g., direct assays or competitive assays described in detail below), or to generate antibodies specific for marker proteins, for example.

Examples of suitable inducible non-fusion E. coli expression vectors include pTrc (Hmann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HSLE174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the polynucleotide sequence of the polynucleotide to be inserted into an expression vector so that the individual codons for each amino acid are those preferentially utilized in E. coli (Wade et al., (1992) Polynucleolides Res. 20:2111-2118). Such alteration of polynucleotide sequences of the invention can be carried out by standard DNA synthesis techniques.

In another embodiment, the CD25⁺ differential marker expression vector is a yeast expression vector. Examples of vectors for expression in yeast S. cerevisiae include pYepSec1 (Baldari, et al., (1987) Embo J. 6:229-234), pMFa (Kurjan and Herskowitz, (1982) Cell 30:933-943), pJRY88 (Schultz et al., 21987) Gene 54:113-123), pYES2 (In Vitrogen Corporation, San Diego, Calif.), and picZ (In Vitrogen Corp, San Diego, Calif.).

Alternatively, marker proteins of the invention can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39).

In yet another embodiment, a polynucleotide of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed.. Cold Spring Expression Technology: Methods in Enzymology 185, Academic Press, San Diego, Calif. (1990) 60-89. Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HSLE174(DE3) from a resident prophage harboring a T7 gn1 gene under the transcriptional control of the lacUV 5 promoter.

In another embodiment, the recombinant mammalian expression vector is capable of directing expression of the polynucleotide preferentially in a particular cell type (e.g., tissue-specific regulatory elements are used to express the polynucleotide). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter, Byrne and Raaddle (1989) Proc. Nall. Acad Sci. USA 86:5473-5477), pancreas-specific promoters (Edlund et al. (1985) Science 230:912-916), and mammary gland-specific promoters (e.g., milk whey promoter, U.S. Pat. No. 4,873,316 and European Application Publication No. 264,166). Developmentally-regulated promoters are also encompassed, for example the marine hox promoters (Kessel and Grass (1990) Science 249:374-379) and the α-fetoprotein promoter (Campes and Tilghman (1989) Genes Dev. 3:537-546). In certain preferred embodiments of the invention, the tissue-specific promoter is a T cell specific promotor.

The invention further provides a recombinant expression vector comprising a CD25⁺ differential marker polynucleotide of the invention cloned into the expression vector in an antisense orientation. That is, the DNA molecule is operatively linked to a regulatory sequence in a manner which allows for expression (by transcription of the DNA molecule) of an RNA molecule which is antisense to mRNA corresponding to a marker gene of the invention (e.g., listed in Table I). Regulatory sequences operatively linked to a polynucleotide cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense polynucleotides are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1)1986.

Another aspect of the invention pertains to host cells into which a polynucleotide molecule of the invention is introduced, e.g., a CD25⁺ differential marker gene listed in Table I, or homolog thereof, within a recombinant expression vector or a polynucleotide molecule of the invention containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a CD25⁺ differential marker protein of the invention can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign polynucleotide (e.g., DNA) into a host cell, including calcium phosphate or calcium chloride co-precipitation, DAKD-dextran-mediated transfection, lipofection, or electoporation. Suitable methods for transforming or transferring host cells can be found in Sambrook, el al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals known in the art.

For stable transfection of mammalian cells, it is known that, depending upon the expression vector and transfection technique used, only a small fraction of cells may integrate the foreign DNA into their genome. In order to identify and select these integrants, a gene that encodes a selectable flag (e.g., resistance to antibiotics) is generally introduced into the host cells along with the gene of interest. Preferred selectable flags include those which confer resistance to drugs, such as G418, hygromycin and methotrexate. Polynucleotide encoding a selectable flag can be introduced into a host cell on the same vector as that encoding a marker protein or can be introduced on a separate vector. Cells stably-transfected with the introduced polynucleotide can be identified by drug selection (e.g., cells that have incorporated the selectable flag gene will survive, while the other cells die).

A host cell of the invention, such as a prokaryotic or eukaryotic host cell in culture, can be used to produce (i.e., express) a marker protein. Accordingly, the invention further provides methods for producing a CD25⁺ differential marker protein using the host cells of the invention. In one embodiment, the method comprises culturing the host cell of invention (into which a recombinant expression vector encoding a marker protein has been introduced) in a suitable medium such that a marker protein of the invention is produced. In another embodiment, the method further comprises isolating a marker protein from the medium or the host cell. In specific embodiments, the CD25⁺ differential marker GITR is produced in the host cell COS or CHO.

The host cells of the invention can also be used to produce non-human transgenic animals. For example, in one embodiment, a host cell of the invention is a fertilized oocyte or an embryonic stem cell into which marker-protein-coding sequences (such as for the marker GITR) have been introduced. Such host cells can then be used to create non-human transgenic animals in which exogenous sequences encoding a marker protein of the invention have been introduced into their genome or homologous recombinant animals in which endogenous sequences encoding the marker proteins of the invention have been altered. Such animals are useful for studying the function and/or activity of a marker protein (such as GITR) and for identifying and/or evaluating modulators of marker protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous marker gene of the invention (e.g., listed in Table I) has been altered by homologous recombination between the endogenous gene and an exogenous DNA molecule introduced into a cell of the animal, e.g., an embryonic cell of the animal, prior to development of the animal.

A transgenic animal of the invention can be created by introducing a marker-encoding polynucleotide into the mate pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to a transgene to direct expression of a marker protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a transgene of the invention in its genome and/or expression of mRNA corresponding to a gene of the invention in tissues or cells of the animals. A transgenic founder animal can then be used to breed additional animals carrying the transgene. Moreover, transgenic animals carrying a transgene encoding a marker protein can further be bred to other transgenic animals carrying other transgenes.

To create a homologous recombinant animal, a vector is prepared which contains at least a portion of a gene of the invention into which a deletion, addition or substitution has been introduced to thereby alter, e.g., functionally disrupt, the gene. The gene can be a human gene, but more preferably, is a non-human homologue of a human gene of the invention (e.g., a homolog of a marker listed in Table I). For example, a mouse gene can be used to construct a homologous recombination polynucleotide molecule, e.g., a vector, suitable far altering an endogenous gene of the invention in the mouse genome. In a preferred embodiment, the homologous recombination polynucleotide molecule is designed such that, upon homologous recombination, the endogenous gene of the invention is functionally disrupted (i.e., no longer encodes a functional protein; also referred to as a “knock out” vector). Alternatively, the homologous recombination polynucleotide molecule can be designed such that, upon homologous recombination, the endogenous gene is mutated or otherwise altered but still encodes functional protein (e.g., the upstream regulatory region can be altered to thereby alter the expression of the endogenous marker protein). In the homologous recombination polynucleotide molecule, the altered portion of the gene of the invention is flanked at its 5′ and 3′ ends by additional polynucleotide sequence of the gene of the invention to allow for homologous recombination to occur between the exogenous gene carried by the homologous recombination polynucleotide molecule and an endogenous gene in a cell, (e.g., an embryonic stem cell) Jul. 9, 2002. The additional flanking polynucleotide sequence is of sufficient length for successful homologous recombination with the endogenous gene.

Typically, several kilobases of flanking DNA (both at the 5′ and 3′ ends) are included in the homologous recombination polynucleotide molecule (see, e.g., Thomas, K. R. and Capecchi, M. R. (1987) Cell 51:503 for a description of homologous recombination vectors). The homologous recombination polynucleotide molecule is introduced into a cell, (e.g., an embryonic stem cell) line (e.g., by electroporation) and cells in which the introduced gene has homologously recombined with the endogenous gene are selected (see e.g., Li, E. et al. (1992) Cell 69:915). The selected cells can then be injected into a blastocyst of an animal (e.g., a mouse) to form aggregation chimeras (see e.g. Bradley, S A. in Teratocareirtomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination polynucleotide molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Laksa et al. (1992) Proc. Natl. Acad. Sci. USA 89:6232-6236. Another example of a recombinase system is the FLP recombinase system of Saccharomyces cerevisiae O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

In preferred embodiments of the invention, the non-human transgenic animals comprise a CD25⁺ differential marker which is GITR. In other preferred embodiments, the non-human “knock-out” transgenic animal is a GITR knock-out.

Detection Methods

Detection and measurement of the relative amount of a polynucleotide or polypeptide marker of the invention may be by any method known in the art (see, i.e., Sambrook, J., Fritsh, E. F., and Maniatis, T. Molecular Cloning: A Laboratory Manual. 2^{nd} ed., Cold Spring Harbor Laboratory, Cold Spring Harbor Laboratory Press, Cold Spring Harbor, N.Y. (1989), and Current Protocols in Molecular Biology, eds. Ausubel et al, John Wiley & Sons (1992)).

Typical methodologies for detection of a transcribed polynucleotide include RNA extraction from a cell or tissue sample, followed by hybridization of a labeled probe (i.e., a complementary polynucleotide molecule) specific for the target RNA to the extracted RNA and detection of the probe (i.e., Northern blotting).

Typical methodologies for peptide detection include protein extraction from a cell or tissue sample, followed by binding of an antibody specific for the target protein to the protein sample, and detection of the antibody. For example, detection of GITR may be accomplished using polyclonal antibody anti-mouse GITR/TNFRSF 18 #AF524 available from R&D Systems (Minneapolis, Minn.). Antibodies are generally detected by the use of a labeled secondary antibody. The label can be a radioisotope, a fluorescent compound, an enzyme, an enzyme co-factor, or ligand. Such methods are well understood in the art.

In certain embodiments, the CD25⁺ differential marker genes themselves (i.e., the DNA or cDNA) may serve as markers for autoimmune disorder. For example, an increase of polynucleotide corresponding to a marker (i.e., a Cluster Type A or Cluster Type B), such as by duplication of the gene, may also be correlated with autoimmune disorder. Similarly, a decrease of polynucleotide corresponding to a marker (i.e., a Cluster Type C or Cluster Type D), such as by deletion of the gene, may also be correlated with autoimmune disorder.

Detection of specific polynucleotide molecules may also be assessed by gel electrophoresis, column chromatography, or direct sequencing, or quantitative PCR (in the case of polynucleotide molecules) among many other techniques well known to those skilled in the art.

Detection of the presence or number of copies of all or a part of a CD25⁺ differential marker gene of the invention may be performed using any method known in the art. Typically, it is convenient to assess the presence and/or quantity of a DNA or cDNA by Southern analysis, in which total DNA from a cell or tissue sample is extracted, is hybridized with a labeled probe (i.e. a complementary DNA molecules), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

In certain embodiments, the CD25⁺ differential marker proteins or polypeptides may serve as markers for an autoimmune disorder. For example, an aberrent increase in the polypeptide corresponding to a marker (i.e., a Cluster Type A or Cluster Type B), may also be correlated with an autoimmune disease. Similarly, an aberrent decrease of a polypeptide corresponding to a marker (i.e., a Cluster Type C or Cluster Type D) may also be correlated with an autoimmune disease.

In other embodiments, the CD25⁺ differential marker proteins or polypeptides may serve as markers for transplant rejection or acceptance. For example, an aberrent increase in the polypeptide corresponding to a marker (i.e., a Cluster Type A or Cluster Type B), may be correlated with transplant rejection. Similarly, an aberrent decrease of a polypeptide corresponding to a marker (i.e., a Cluster Type C or Cluster Type D) may also be correlated with transplant rejection.

Detection of specific polypeptide molecules may also be assessed by gel electrophoresis, column chromatography, or direct sequencing, among many other techniques well known to those skilled in the art.

Panels of CD25⁺ Differential Markers

Each marker may be considered individually, although it is within the scope of the invention to provide combinations of two or more markers for use in the methods and compositions of the invention to increase the confidence of the analysis. In another aspect, the invention provides panels of the CD25⁺ differential markers of the invention. A panel of markers comprises 5 or more CD25⁺ differential markers. A panel may also comprise 5-15, 15-35, 35-50, 50-100, or more than 100 CD25⁺ differential markers. In a preferred embodiment, these panels of markers are selected such that the markers within any one panel share certain features. For example, the markers of a first panel may each exhibit at least a two-fold increase in quantity or activity in an autoimmune sample, as compared to a sample which is substantially free of the autoimmune disorder, from the same subject or a sample which is substantially free of the autoimmune disorder from a different subject without said autoimmune disorder. Alternatively, markers of a second panel may each exhibit differential regulation as compared to a first panel. Similarly, different panels of markers may be composed of markers from different Functional Categories, Cluster Types, or samples (i.e., kidney, spleen, node, brain, heart or urine), or may be selected to represent different stages of an autoimmune disorder. Panels of the CD25⁺ differential markers of the invention may be made by independently selecting markers from Table I, and may further be provided on biochips, as discussed below.

Screening

The invention also provides methods (also referred to herein as “screening assays”) for identifying modulators, (i.e., candidate or test compounds or agents) comprising therapeutic moieties (e.g., peptides, peptidomimetics, peptoids, polynucleotides, small molecules or other drugs) which (a) bind to the marker, or (b) have a modulatory (e.g., stimulatory or inhibitory) effect on the activity of a CD25⁺ differential marker or, more specifically, (c) have a modulatory effect on the interactions of the marker with one or more of its natural substrates (e.g., peptide, protein, hormone, co-factor, or polynucleotide), or (d) have a modulatory effect on the expression of the marker. Such assays typically comprise a reaction between the marker and one or more assay components. The other components may be either the test compound itself, or a combination of test compound and a binding partner of the marker.

The test compounds of the present invention are generally either small molecules or bioactive agents. In one preferred embodiment the test compound is a small molecule. In another preferred embodiment, the test compound is a bioactive agent. Bioactive agents include but are not limited to naturally-occurring or synthetic compounds or molecules (“biomolecules”) having bioactivity in mammals, as well as proteins, peptides, oligopeptides, polysaccharides, nucleotides and polynucleotides. Preferably, the bioactive agent is a protein, polynucleotide or biomolecule. One skilled in the art will appreciate that the nature of the test compound may vary depending on the nature of the protein encoded by the marker of the invention. For example, if the marker encodes an orphan receptor having an unknown ligand, the test compound may be any of a number of bioactive agents which may act as cognate ligand, including but not limited to, cytokines, lipid-derived mediators, small biogenic amines, hormones, neuropeptides, or proteases.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

As used herein, the term “specific factor” refers to a bioactive agent which serves as either a substrate for a protein encoded by a CD25⁺ differential marker of the invention, or alternatively, as a ligand having binding affinity to the protein or a binding partner for a CD25⁺ differential marker. As mentioned above, the bioactive agent may be any of a variety of naturally-occurring or synthetic compounds, biomolecules, proteins, peptides, oligopeptides, polysaccharides, nucleotides or polynucleotides.

Screening for Inhibitors of Autoimmune Disorders or Transplant Rejection

The invention provides methods of screening test compounds for inhibitors of autoimmune disorders, and to the pharmaceutical compositions comprising the test compounds. The invention also provides methods of screening test compounds for inhibitors of transplant rejection, and to the pharmaceutical compositions comprising the test compounds. The method of screening comprises obtaining samples from subjects diagnosed with or suspected of having an autoimmune disorder or transplant rejection, contacting each separate aliquot of the samples with one of a plurality of test compounds, and comparing expression of one or more CD25⁺ differential marker(s) in each of the aliquots to determine whether any of the test compounds provides: 1) a substantially decreased level of expression or activity of a Cluster Type A or Cluster Type B marker, or 2) a substantially increased level of expression or activity of a Cluster Type C or Cluster Type D, marker relative to samples with other test compounds or relative to an untreated sample or control sample. In addition, methods of screening may be devised by combining a test compound with a protein and thereby determining the effect of the test compound on the protein.

In addition, the invention is further directed to a method of screening for test compounds capable of modulating with the binding of a protein encoded by the CD25⁺ differential markers of Table I and a specific factor, by combining the test compound, protein, and specific factor together and determining whether binding of the specific factor and protein occurs. The test compound may be either small molecules or a bioactive agent. As discussed below, test compounds may be provided from a variety of libraries well known in the art.

In a specific embodiments the screening assay involves detection of a test compound's ability to modulate with the binding of a GITR-ligand to GITR or detection of a test compound's ability to lead to signaling or cause signaling through GITR. Such compounds may provide therapeutic agents of the invention useful for the treatment of autoimmune diseases, e.g. rheumatoid arthritis.

Modulators of a CD25⁺ differential marker expression, activity or binding ability are useful as thereapeutic compositions of the invention. Such modulators (e.g., antagonists or agonists) may be formulated as pharmaceutical compositions, as described herein below. Such modulators may also be used in the methods of the invention, for example, to diagnose, treat, or prognose an autoimmune disorder or transplant rejection.

Screening for Inhibitors of Cancer or Proliferative Disorders

The invention also provides methods of screening test compounds for inhibitors of suppressor T cells which are thereby inhibitors of cancer or proliferative disorder. This method of screening comprises obtaining samples from subjects diagnosed with or suspected of having cancer or a proliferative disorder, containing separate aliquots of the sample with one of a plurality of test compounds, and comparing expression of one or more CD25⁺ differential marker(s) in each of the aliquots to determine whether any of the test compounds provides 1) a substantially increased level of expression or activity of a Cluster Type A or Cluster Type B marker or 2) a substantially decreased level of expression of a Cluster Type C or a Cluster Type D marker, relative to samples with other test compounds or relative to an untreated sample or control sample.

High-Throughput Screening Assays

The invention provides methods of conducting high-throughput screening for test compounds capable of inhibiting activity or expression of a protein encoded by a CD25⁺ differential markers of the invention. In one embodiment, the method of high-throughput screening involves combining test compounds and the marker protein and detecting the effect of the test compound on the encoded protein. Functional assays such as cytosensor microphysiometer, calcium flux assays such as FLIPR® (Molecular Devices Corp, Sunnyvale, Calif.), or the TUNEL assay may be employed to measure cellular activity, as discussed below.

A variety of high-throughput functional assays well-known in the art may be used in combination to screen and/or study the reactivity of different types of activating test compounds, but since the coupling system is often difficult to predict a number of assays may need to be configured to detect a wide range of coupling mechanisms. A variety of fluorescence-based techniques are well-known in the art and are capable of high-throughput and ultra high throughput screening for activity, including but not limited, to BRET® or FRET® (both by Packard Instrument Co., Meriden, Conn.). A preferred high-throughput screening assay is provided by BIACORE® systems, which utilizes label-free surface plasmon resonance technology to detect binding between a variety of bioactive agents, as described in further detail below. The ability to screen a large volume and a variety of test compounds with great sensitivity permits for analysis of the therapeutic targets of the invention to further provide potential inhibitors of autoimmune disorders or cancer. For example, where the marker encodes an orphan receptor with an unidentified ligand, high-throughput assays may be utilized to identify the ligand, and to further identify test compounds which prevent binding of the receptor to the ligand. The BIACORE® system may also be manipulated to detect binding of test compounds with individual components of the therapeutic target, to detect binding to either the encoded protein or to the ligand.

Recent advancements have provided a number of methods to detect binding activity between bioactive agents. Common methods of high-throughput screening involve the use of of fluorescence-based technology, including but not limited, to BRET® or FRET® (both by Packard Instrument Co., Meriden, Conn.) which measure the detection signal provided by the proximity of bound fluorophores. By combining test compounds with proteins encoded by the markers of the invention and determining the binding activity between such, diagnostic analysis can be performed to elucidate the coupling systems. Generic assays using cytosensor microphysiometer may also be used to measure metabolic activation, while changes in calcium mobilization can be detected by using the fluorescence-based techniques such as FLIPR® (Molecular Devices Corp, Sunnyvale, Calif.). In addition, the presence of apoptotic cells may be determined by TUNEL assay, which utilizes flow cytometry to detect free 3-OH termini resulting from cleavage of genomic DNA during apoptosis. As mentioned above, a variety of functional assays well-known in the art may be used in combination to screen and/or study the reactivity of different types of activating test compounds. Preferably, the high-throughput screening assay of the present invention utilizes label-free plasmon resonance technology as provided by BIACORE® systems (Biacore International AB, Uppsala, Sweden). Plasmon free resonance occurs when surface plasmon waves are excited at a metal/liquid interface. By reflecting directed light from the surface as a result of contact with a sample, the surface plasmon resonance causes a change in the refractive index at the surface layer. The refractive index change for a given change of mass concentration at the surface layer is similar for many bioactive agents (including proteins, peptides, lipids and polynucleotides), and since the BIACORE® sensor surface can be functionalized to bind a variety of these bioactive agents, detection of a wide selection of test compounds can thus be accomplished.

Therefore, the invention provides for high-throughput screening of test compounds for the ability to inhibit activity of a protein encoded by the markers listed in Table I, by combining the test compounds and the protein in high-throughput assays such as BIACORE®, or in fluorescence based assays such as BRET®. In addition, high-throughput assays may be utilized to identify specific factors which bind to the encoded proteins, or alternatively, to identify test compounds which prevent binding of the receptor to the specific factor. In the case of orphan receptors, the specific factor may be the natural ligand for the receptor. Moreover, the high-throughput screening assays may be modified to determine whether test compounds can bind to either the encoded protein or to the specific factor (e.g. substrate or ligand) which binds to the protein.

In a specific embodiment, the high-throughput screening assay detects the ability of a plurality of test compounds to bind to GITR. In another specific embodiment, the high-throughput screening assay detects the ability of a plurality of a test compound to inhibit a GITR binding partner (such as GITR ligand) to bind to GITR. In yet another specific embodiment, the high-throughput screening assay detects the ability of a plurality of a test compounds to modulate signaling through GITR.

Predictive Medicine

The present invention pertains to the field of predictive medicine in which diagnostic assays, prognostic assays, pharmacogenetics and monitoring clinical trials are used for prognostic (predictive) purposes to thereby treat an individual prophylactically. Accordingly, one aspect of the present invention relates to diagnostic assays for determining CD25⁺ differential marker polynucleotide and/or polypeptide expression and/or activity, in the context of a biological sample (e.g., blood, serum, cells, tissue) to thereby determine whether an individual is at risk for developing an autoimmune disorder associated with modulated marker expression or activity. The invention also provides for prognostic (or predictive) assays for determining whether an individual is at risk of developing an autoimmune disorder associated with aberrant marker protein or polynucleotide expression or activity. The invention also provides for prognostic (or predictive) assays for determining whether an individual is at risk of developing transplant rejection associated with aberrant marker protein or polynucleotide expression or activity.

For example, the number of copies of a marker gene can be assayed in a biological sample. Such assays can be used for prognostic or predictive purposes to thereby phophylactically treat an individual prior to the onset of an autoimmune disease (or acute rejection in transplants), characterized by or associated with aberrant marker protein, polynucleotide expression or activity.

Another aspect of the invention pertains to monitoring the influence of agents (e.g., drugs, compounds) on the expression or activity of marker in clinical trials.

Diagnostic Assays

An exemplary method for detecting the presence or absence of marker protein or polynucleotide of the invention in a biological sample involves obtaining a biological sample from a test subject and contacting the biological sample with a compound or an agent capable of detecting the protein or polynucleotide (e.g., mRNA, genomic DNA) that encodes the marker protein such that the presence of the marker protein or polynucleotide is detected in the biological sample. A preferred agent for detecting mRNA or genomic DNA corresponding to a marker gene or protein of the invention is a labeled polynucleotide probe capable of detecting to a mRNA or genomic DNA of the invention. Suitable probes for use in the diagnostic assays of the invention are described herein. A preferred agent for detecting a marker protein of the invention is an antibody which specifically recognizes the marker.

The diagnostic assays may also be used to quantify the amount of expression or activity of a CD25⁺ differential marker in a biological sample. Such quantification is useful, for example, to determine the progression or severity of a autoimmune disorder or transplant rejection. Such quantification is also useful, for example, to determine the severity of a cancer or the regression of a cancer or proliferative disorder following treatment.

Determining Severity of an Autoimmune Disease

In the field of diagnostic assays, the invention also provides methods for determining the severity of an autoimmune disease by isolating a sample from a subject (e.g., a blood sample containing T cells), detecting the presence, quantity and/or activity of one or more markers of the invention in the sample relative to a second sample from a normal sample or control sample. In one embodiment, the levels of markers in the two samples are compared, and a modulation in one or more markers in the test sample indicates an autoimmune disorder. In other embodiments the modulation of 2, 3, 4 or more markers indicate a severe autoimmune disorder.

In another aspect, the invention provides markers whose quantity or activity is correlated with different manifestations or severity or type of autoimmune disorder, including, in the field of rheumatoid arthritis, the onset of joint pain. In certain embodiments, these markers have modulated quantity or activity in a fashion that is correlated with the degree of severity of joint inflammation which may in turn indicate permanent tissue damage. The subsequent level of expression may further be compared to different expression profiles of various stages of the disorder to confirm whether the subject has a matching profile. In yet another aspect, the invention provides CD25⁺ differential markers whose quantity or activity is correlated with a risk in a subject for developing autoimmune disorder.

A preferred agent for detecting marker protein is an antibody capable of binding to marker protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect marker mRNA, protein, or genomic DNA in a biological sample in vitro as well as in vivo. For example, in vitro techniques for detection of marker mRNA include Northern hybridizations and in situ hybridizations. In vitro techniques for detection of marker protein include enzyme linked immunosorbent assays (ELISAs), Western blots, immunoprecipitations and immunofluorescence. In vitro techniques for detection of marker genomic DNA include Southern hybridizations. Furthermore, in vivo techniques for detection of marker protein include introducing into a subject a labeled anti-marker antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a subject, contacting the control sample with a compound or agent capable of detecting marker protein, mRNA, or genomic DNA, such that the presence of marker protein, mRNA or genomic DNA is detected in the biological sample, and comparing the presence of marker protein, mRNA or genomic DNA in the control sample with the presence of marker protein, mRNA or genomic DNA in the test sample.

The invention also encompasses kits for detecting the presence of CD25⁺ differential marker in a biological sample. For example, the kit can comprise a labeled compound or agent capable of detecting marker protein or mRNA in a biological sample; means for determining the amount of marker in the sample; and means for comparing the amount of marker in the sample with a control or standard. The compound or agent can be packaged in a suitable container. The kit can further comprise instructions for using the kit to detect marker protein or polynucleotide.

Prognostic Assays

The diagnostic methods, described herein can furthermore be utilized to identify subjects having or at risk of developing an autoimmune disorder or transplant rejection associated with aberrant marker expression or activity. In one embodiment of the present invention, as related to an autoimmune disorder or transplant rejection, aberrant expression or activity of Cluster Type A or Cluster Type B markers is typically correlated with an abnormal increase. In another embodiment of the present invention, as related to an autoimmune disorder or transplant rejection, aberrant expression or activity of Cluster Type C or Cluster Type D markers is typically correlated with an abnormal decrease.

The assays described herein, such as the preceding or following assays, can be utilized to identify a subject having an autoimmune disorder or transplant rejection associated with an aberrant level of marker activity or expression. Alternatively, the prognostic assays can be utilized to identify a subject at risk for developing an autoimmune disorder associated with aberrant levels of marker protein activity or polynucleotide expression. Thus, the present invention provides a method for identifying autoimmune disorders associated with aberrant marker expression or activity in which a test sample is obtained from a subject and marker protein or polynucleotide (e.g., mRNA or genomic DNA) is detected, wherein the presence of marker protein or polynucleotide is diagnostic or prognostic for a subject having or at risk of developing transplant rejection with aberrant marker expression or activity.

Furthermore, the prognostic assays described herein can be used to determine whether a subject can be administered an agent (e.g., an agonist, antagonist, peptidomimetic, protein, peptide, polynucleotide, small molecule, or other drug candidate) to treat or prevent an autoimmune disorder, transplant rejection or cancer associated with aberrant marker expression or activity. For example, such methods can be used to determine whether a subject can be effectively treated with an agent to inhibit an autoimmune disorder. Thus, the present invention provides methods for determining whether a subject can be effectively treated with an agent for a disorder associated with increased marker expression or activity in which a test sample is obtained and marker protein or polynucleotide expression or activity is detected (e.g, wherein the abundance of marker protein or polynucleotide expression or activity is diagnostic for a subject that can be administered the agent to treat injury associated with aberrant marker expression or activity).

In relation to the field of autoimmune disorder, prognostic assays can be devised to determine whether a subject undergoing treatment for such disorder has a poor outlook for long term survival or disease progression. In a preferred embodiment, prognosis can be determined shortly after diagnosis, i.e., within a few days. By establishing expression profiles of different stages of the autoimune disorder, from onset to acute disease, an expression pattern may emerge to correlate a particular expression profile to increased likelihood of a poor prognosis. The prognosis may then be used to devise a more aggressive treatment program to avert a chronic autoimmune disorder and enhance the likelihood of long-term survival and well being. Similarly, such prognostic assays can be devised for the progression of transplant rejection or acceptance. By establishing expression profiles of different stages of the immune response following transplant, e.g., from transplant to acute rejection, an expression pattern may emerge to correlate a particular expression profile to increased likelihood of a poor prognosis. The prognosis may then be used to devise a more aggressive treatment program to avert acute rejection and enhance the likelihood of long-term survival following transplant.

Similarly, in relation the field of cancer and proliferative disorders, prognostic assays can be devised to determine whether a subject undergoing treatment for such a disorder has a poor outlook for long term survival or disease progression. For example, by establishing expression profiles of different stages of the cancer or proliferative disorder, from onset to acute disease, an expression pattern may emerge to correlate a particular expression profile to an increased likelihood of a poor prognosis. Such a prognosis may then be used to devise a more aggressive treatment program to avert a chronic or malignant cancer and enhance the chances of long term survival.

The methods of the invention can also be used to detect genetic alterations in a marker gene, thereby determining if a subject with the altered gene is at risk for damage characterized by aberrant regulation in marker protein activity or polynucleotide expression. In preferred embodiments, the methods include detecting, in a sample of cells from the subject, the presence or absence of a genetic alteration characterized by at least one alteration affecting the integrity of a gene encoding a marker-protein, or the aberrant expression of the marker gene. For example, such genetic alterations can be detected by ascertaining the existence of at least one 1) deletion of one or more nucleotides from a marker gene; 2) addition of one or more nucleotides to a marker gene; 3) substitution of one or more nucleotides of a marker gene, 4) a chromosomal rearrangement of a marker gene; 5) alteration in the level of a messenger RNA transcript of a marker gene, 6) aberrant modification of a marker gene, such as of the methylation pattern of the genomic DNA, 7) the presence of a non-wild type splicing pattern of a messenger RNA transcript of a marker gene, 8) non-wild type level of a marker-protein, 9) allelic loss of a marker gene, and 10) inappropriate post-translational modification of a marker-protein. As described herein, there are a large number of assays known in the art which can be used for detecting alterations in a marker gene. A preferred biological sample is a blood sample isolated by conventional means from a subject. In a specific embodiment, the marker gene detected is GITR. In a preferred embodiment, the marker gene detected is human GITR.

In certain embodiments, detection of the alteration involves the use of a probe/primer in a polymerase chain reaction (PCR) (see, e.g., U.S. Pat. Nos. 4,683,(95 and 4,683,202), such as anchor PCR or RACE PCR, or, alternatively, in a ligation chain reaction (LCR) (see, e.g., Landegran el al. (1988) Science 241:1077-1080; and Nakazawa el al. (1994) Proc. Mail. Acad. Sci. USA 91:360-364), the latter of which can be particularly useful for detecting point mutations in the marker-gene (see Abravaya et al. (1995) Polynucleotides Res. 23:675-682). This method can include the steps of collecting a sample of cells from a subject, isolating polynucleotide (e.g., genomic, mRNA or both) from the cells of the sample, contacting the polynucleotide sample with one or more primers which specifically hybridize to a marker gene under conditions such that hybridization and amplification of the marker-gene (if present) occurs, and detecting the presence or absence of an amplification product, or detecting the size of the amplification product and comparing the length to a control sample. It is understood that PCR and/or LCR may be desirable to use as a preliminary amplification step in conjunction with any of the techniques used for detecting mutations described herein.

Alternative amplification methods include: self sustained sequence replication (Guatelli, J C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other polynucleotide amplification method, followed by the detection of the amplified molecules using techniques well known to those of skill in the art. These detection schemes are especially useful for the detection of polynucleotide molecules if such molecules are present in very low numbers.

In an alternative embodiment, mutations in a CD25⁺ differential marker gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in a CD25⁺ differential marker gene or a gene encoding a CD25⁺ differential marker protein of the invention can be identified by hybridizing a sample and control polynucleotides, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in marker can be identified in two dimensional arrays containing light generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the marker gene and detect mutations by comparing the sequence of the sample marker with the corresponding wild-type (control) sequence. Examples of sequencing reactions include those based on techniques developed by Maxam and Gilbert ((1977) Proc. Natl. Acad. Scl. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94116101; Cohen et al. (1996) Adv. Chromatogr. 36:127-162; and Griffin et al. (1993) Appl. Biochem. Biotechnol. 38:147-159).

Other methods for detecting mutations in a CD25⁺ differential marker gene or gene encoding a marker protein of the invention include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes by hybridizing (labeled) RNA or DNA containing the wild-type marker sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397; Saleeba et al. (1992) Methods Enzymol. 517:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in marker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1652). According to an exemplary embodiment, a probe based on a marker sequence, e.g., a wild-type marker sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in marker genes or genes encoding a marker protein of the invention. For example, single strand conformation polymorphism (SSCP) may be used to detect differences in electrophoretic mobility between mutant and wild type polynucleotides (Orita et al. (1989) Proc Natl. Acad. Sci USA: 86:2766, see also Cotton (1993) Mutat. Res. 285:125-144; and Hayashi (1992) Genet. Anal. Tech Appl. 9:73-79). Single-stranded DNA fragments of sample and control marker polynucleotides will be denatured and allowed to renature. The secondary structure of single-stranded polynucleotides varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Polynucleotides Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing prepackaged diagnostic kits comprising at least one probe polynucleotide or antibody reagent described herein, which may be conveniently used, e.g., in clinical settings to diagnose subjects exhibiting symptoms or family history of a disease or illness involving a marker gene. In a specific embodiment of the invention, a mutation is detected in a GITR polynucleotide or GITR polypeptide. In a further specific embodiment, such GITR mutation is correlated with the prognosis or susceptibility of a subject to an autoimmune disorder such as rheumatoid arthritis; systemic lupus erythematosis; psoriasis; multiple sclerosis; insulin-dependent diabetes mellitus (type I diabetes); inflammatory bowel disease including ulcerative colitis and Crohn's disease (regional enteritis); asthma; or allertic rhinitis.

Furthermore, any cell type or tissue in which a CD25⁺ differential marker is expressed may be utilized in the prognostic or diagnostic assays described herein.

Monitoring of Effects During Clinical Trials

Monitoring the influence of agents (e.g., drugs, small molecules, proteins, nucleotides) on the expression or activity of a marker protein (e.g., the modulation of a CD25⁺ differential marker involved in autoimmune disorder, transplant rejection or cancer) can be applied not only in basic drug screening, but also in clinical trials. For example, the effectiveness of an agent determined by a screening assay, as described herein to decrease marker gene expression, protein levels, or downregulate marker activity, can be monitored in clinical trials of subjects exhibiting increased marker gene expression, protein levels, or upregulated marker activity. Similarly, the effectiveness of an agent to increase marker gene expression, protein levels, or upregulate marker activity can be monitored in clinical trials of subjects exhibiting decreased marker gene expression, protein levels or down-regulated marker activity. In such clinical trials, the expression or activity of a marker gene, and preferably, other genes that have been implicated in, for example, marker-associated damage (e.g., resulting from autoimmune disorder) can be used as a “read out” of the phenotype of a particular cell.

For example, and not by way of limitation, genes, including marker genes and genes encoding a marker protein of the invention, that are modulated in cells by treatment with an agent which modulates marker activity (e.g., identified in a screening assay as described herein) can be identified. Thus, to study the effect of agents on marker-associated damage (e.g., resulting an autoimmune disorder), for example, in a clinical trial, cells can be isolated and RNA prepared and analyzed for the levels of expression of marker and other genes implicated in the marker-associated damage, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of marker or other genes. In this way, the gene expression pattern can serve as a read-out, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, polynucleotide, small molecule, or other drug candidate identified by the screening assays described herein) including the steps of: (i) obtaining a pre-administration sample from a subject prior to administration of the agent; (ii) detecting the level of expression of a CD25⁺ differential marker protein, mRNA, or genomic DNA in the pre-administration sample; (iii) obtaining one or more post-administration samples from the subject; (iv) detecting the level of expression or activity of the marker protein, mRNA, or genomic DNA in the post-administration samples; (v) comparing the level of expression or activity of the marker protein, mRNA, or genomic DNA in the pre-administration sample with the marker protein, mRNA, or genomic DNA in the post administration sample or samples; and (vi) altering the administration of the agent to the subject accordingly. For example, decreased administration of the agent may be desirable to decrease expression or activity of marker to lower levels than detected, i.e. to decrease the effectiveness of the agent. According to such an embodiment, marker expression or activity may be used as an indicator of the effectiveness of an agent, even in the absence of an observable phenotypic response.

Methods of Treatment

The present invention provides for both prophylactic and therapeutic methods of treating a subject at risk for, susceptible to or diagnosed with an autoimmune disorder or transplant rejection. The invention also provides for both prophylactic and therapeutic methods of treating a subject at risk for, susceptible to, or diagnosed with cancer or a proliferation disorder. With regards to both prophylactic and therapeutic methods of treatment, such treatments may be specifically tailored or modified, based on knowledge obtained from the field of pharmacogenomics. “Pharmacogenomics”, as used herein, includes the application of genomics technologies such as gene sequencing, statistical genetics, and gene expression analysis to drugs in clinical development and on the market. More specifically, the term refers the study of how a subject's genes determine his or her response to a drug (e.g., a subject's “drug response phenotype”, or “drug response genotype”). Thus, another aspect of the invention provides methods for tailoring an individual's prophylactic or therapeutic treatment with either the marker molecules of the present invention or marker modulators (e.g., agonists or antagonists) according to that individual's drug response. Pharmacogenomics allows a clinician or physician to target prophylactic or therapeutic treatments to subjects who will most benefit from the treatment and to avoid treatment of subjects who will experience toxic drug-related side effects.

Prophylactic Methods

In one aspect, the invention provides a method for preventing in a subject, an autoimmune disorder associated with aberrant CD25⁺ differential marker expression or activity, by administering to the subject a marker protein or an agent which modulates marker protein expression or activity. In another aspect, the invention provides a method for preventing in a subject, transplant rejection associated with aberrant CD25⁺ differential marker expression or activity, by administering to the subject a marker protein or an agent which modulates marker protein expression or activity.

Subjects at risk for a disease which is caused or contributed to by aberrant marker expression or activity can be identified by, for example, any or a combination of diagnostic or prognostic assays as described herein.

Administration of a prophylactic agent can occur prior to the manifestation of symptoms characteristic of the differential marker protein expression, such that the autoimmune disorder is prevented or, alternatively, delayed in its progression. Depending on the type of marker aberrancy (e.g., typically a modulation outside the normal standard deviation), for example, a marker protein, marker agonist or antagonist agent can be used for treating the subject. The appropriate agent can be determined based on screening assays described herein.

In another aspect, the invention provides a method for preventing in a subject a cancer or proliferative disorder by administering to the subject a marker protein or agent which modulates marker protein expression or activity. One of skill in the art will appreciate that with respect to embodiments for treating or preventing cancer or proliferative disorders, therapeutic or prophylactic methods generally seek to inhibit suppressor T cells and the differential expression associated with CD25⁺ T cells. As such, agonists or antagonists of CD25⁺ differential markers may be administered to effectuate expression of CD25⁺ differential markers which are similar or substantially similar to CD25⁻ T cells. Appropriate agents for such use may be determined based on screening assays described herein.

Therapeutic Methods

Another aspect of the invention pertains to methods of modulating marker protein expression or activity for therapeutic purposes. Accordingly, in an exemplary embodiment, the modulatory method of the invention involves contacting a cell with a CD25⁺ diferential marker (such as GITR) marker protein or agent that modulates one or more of the activities of a marker protein activity associated with the cell. An agent that modulates marker protein activity can be an agent as described herein, such as a polynucleotide or a protein, a naturally-occurring target molecule of a marker protein (e.g., a marker protein substrate), a marker protein antibody, a marker modulator (e.g., agonist or antagonist), a peptidomimetic of a marker protein agonist or antagonist, or other small molecule.

In one embodiment, the agent stimulates one or more marker protein activities. Examples of such stimulatory agents include active marker protein and a polynucleotide molecule encoding marker protein that has been introduced into the cell. In a specific embodiment, GITR ligand is used to stimulate activity of GITR.

In another embodiment, the agent inhibits one or more marker protein activities. Examples of such inhibitory agents include antisense marker protein nucleic said molecules, anti-marker protein antibodies, and marker protein inhibitors. In a specific embodiment, an inhibitor of agent is an anti-sense GITR polynucleotide.

These modulatory methods can be performed in vitro (e.g., by culturing the cell with the agent) or, alternatively, in vivo (e.g., by administering the agent to a subject). As such, the present invention provides methods of treating an individual diagnosed with or at risk for an autoimmune disorder characterized by aberrant expression or activity of one or more CD25⁺ differential marker proteins or polynucleotide molecules. In one embodiment, the method involves administering an agent (e.g., an agent identified by a screening assay described herein), or combination of agents that modulates (e.g., upregulates or downregulates) marker protein expression or activity. In another embodiment, the method involves administering a marker protein or polynucleotide molecule as therapy to compensate for reduced or aberrant marker protein expression or activity.

Stimulation of marker protein activity is desirable in situations in which marker protein is abnormally downregulated and/or in which increased marker protein activity is likely to have a beneficial effect. Likewise, particularly with regards to the markers listed in Table I, which are differentially expressed in CD25⁺ T cells, alteration of marker protein or activity to levels similar to CD25⁺ T cells is likely to have a beneficial effect with respect to autoimmune disorders. Alteration of marker protein or activity to levels similar to CD25⁻ T cells is likely to have a beneficial effect with respect to cancer or proliferative disorders.

The invention further provides methods of modulating a level of expression of a CD25⁺ differential marker of the invention, comprising administration to a subject having an autoimmune disorder or cancer a variety of compositions which correspond to the markers of Table I, including proteins or antisense oligonucleotides. The protein may be provided by further providing a vector comprising a polynucleotide encoding the protein to the cells. Alternatively, the expression levels of the markers of the invention may be modulated by providing an antibody, a plurality of antibodies or an antibody conjugated to a therapeutic moiety. Treatment with the antibody may further be localized to the autoimmune tissue comprising the disorder. In another aspect, the invention provides methods for localizing a therapeutic moiety to autoimmune tissue or cells comprising exposing the tissue or cells to an antibody which is specific to a protein encoded from the markers of the invention. This method may therefore provide a means to inhibit or enhance expression of a specific gene corresponding to a marker listed in Table I. Where the gene is up-regulated as a result of an autoimmune disorder (such as Cluster Type A or Cluster Type B), it is likely that inhibition or prevention of the disorder would involve inhibiting expression of the up-regulated gene. Where the gene is down-regulated as a result of an autoimmune disorder (such as Cluster Type C or Cluster Type D), it is likely that inhibition or prevention of the disorder would involve enhancing expression of the down-regulated gene.

Determining Efficacy of a Test Compound or Therapy

The invention also provides methods of assessing the efficacy of a test compound or therapy for inhibiting an autoimmune disorder or transplant rejection in a subject. These methods involve isolating samples from a subject suffering from an autoimmune disorder or transplant rejection, who is undergoing treatment or therapy, and detecting the presence, quantity, and/or activity of one or more markers of the invention in the first sample relative to a second sample. Where a test compound is administered, the first and second samples are preferably sub-portions of a single sample taken from the subject, wherein the first portion is exposed to the test compound and the second portion is not. In one aspect of this embodiment, the CD25⁺ differential marker is expressed at a substantially decreased level in the first sample, relative to the second. Most preferably, the level of expression in the first sample approximates (i.e., is less than the standard deviation for normal samples) the level of expression in a third control sample, taken from a control sample of normal tissue. In another aspect of this embodiment, the CD25⁺ differential marker is expressed at a substantially increased level in the first sample, relative to the second. Most preferably, the level of expression in the first sample approximates (i.e., is less than the standard deviation for normal samples) the level of expression in a third control sample, taken from a control sample of normal tissue.

In certain embodiments, the normal sample is a CD25⁻ T cell. In other embodiments the normal sample is derived from a tissue substantially free of an autoimmune disorder or transplant rejection.

Where the efficacy of a therapy is being assessed, the first sample obtained from the subject is preferably obtained prior to provision of at least a portion of the therapy, whereas the second sample is obtained following provision of the portion of the therapy. The levels of markers in the samples are compared, preferably against a third control sample as well, and correlated with the presence, risk of presence, or severity of the autoimmune disorder. Most preferably, the level of markers in the second sample approximates the level of expression of a third control sample. In the present invention, a substantially decreased level of expression of a marker indicates that the therapy is efficacious for treating the autoimmune disorder.

Pharmacogenomics

The marker protein and polynucleotide molecules of the present invention, as well as agents, inhibitors or modulators which have a stimulatory or inhibitory effect on a CD25⁺ differential marker as identified by a screening assay described herein, can be administered to individuals to treat (prophylactically or therapeutically) marker-associated auto-immune disorders associated with aberrant marker protein activity. The marker protein and polynucleotides of the present invention as well as agents, inhibitors or modulators which have a stimulatory or inhibitory effect on a CD25⁺ differential marker can also be administered to individuals to treat (prophylactically or therapeutically) a cancer or proliferative disorder.

In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a marker molecule or marker modulator as well as tailoring the dosage and/or therapeutic regimen of treatment with a marker molecule or marker modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linden, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related sites (e.g., a “bi-allelic” gene marker map which consists of 60,000-100,000 polymorphic or variable sites on the human genome, each of which has two variants.) Such a high-resolution genetic map can be compared to a map of the genome of each of a statistically substantial number of subjects taking part in a Phase II/III drug trial to identify genes associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug target is known (e.g., a CD25⁺ differential marker protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYPZC19) has provided an explanation as to why some subjects do not obtain the expected drug effects or show exaggerated drug response and serious toxicity after taking the standard and safe dose of a drug. These polymorphisms are expressed in two phenotypes in the population, the extensive metabolizer and poor metabolizer. The prevalence of poor metabilizer phenotypes is different among different populations. For example, the gene coding for CYP2D6 is highly polymorphic and several mutations have been identified in poor metabilizers, which all lead to the absence of functional CYP2D6. Poor metabolizers of CYP2D6 and CYP2C19 quite frequently experience exaggerated drug response and side effects when they receive standard doses. If a metabolite is the active therapeutic moiety, poor metabilizers show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., a marker or marker modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a marker or marker modulator, such as a modulator identified by one of the exemplary screening assays described herein.

Pharmaceutical Compositions

The invention is further directed to pharmaceutical compositions comprising the test compound, or bioactive agent, or a marker modulator (i.e., agonist or antagonist), which may further include a marker protein and/or polynucleotide of the invention (e.g., for those markers in Table I which are differentially expressed in CD25⁺ T cells versus CD25⁻ T cells), and can be formulated as described herein. Alternatively, these compositions may include an antibody which specifically binds to a CD25⁺ differential marker protein of the invention and/or an antisense polynucleotide molecule which is complementary to a CD25⁺ differential marker polynucleotide of the invention (e.g., for those markers which are increased in quantity) and can be formulated as described herein.

One or more of the CD25⁺ differential marker genes (listed in Table I) of the invention fragments of marker genes, marker proteins, marker modulators, fragments of marker proteins, or anti-marker protein antibodies of the invention can be incorporated into pharmaceutical compositions suitable for administration.

As used herein the language “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. See e.g. A. H. Kibbe Handbook of Pharmaceutical Excipients, 3rd ed. Pharmaceutical Press, London, UK (2000). Except insofar as any conventional media or agent is incompatible with the active compound, use thereof in the compositions is contemplated. Supplementary agents can also be incorporated into the compositions.

The invention includes methods for preparing pharmaceutical compositions for modulating the expression or activity of a polypeptide or polynucleotide corresponding to a marker of the invention. Such methods comprise formulating a pharmaceutically acceptable carrier with an agent which modulates expression or activity of a polypeptide or polynucleotide corresponding to a marker of the invention. Such compositions can further include additional active agents. Thus, the invention further includes methods for preparing a pharmaceutical composition by formulating a pharmaceutically acceptable carrier with an agent which modulates expression or activity of a polypeptide or polynucleotide corresponding to a marker of the invention and one or more additional bioactive agents.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The earner can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a marker protein or an anti-marker protein antibody) in the required amount in an appropriate solvent with one or a combination of ingredients enmnerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active, ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, (e.g., a gas such as carbon dioxide, or a nebulizer).

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bioactive compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the therapeutic moieties, which may contain a bioactive compound, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from e.g, Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on-the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The CD25⁺ differential polynucleotide molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Kits

The invention also provides kits for determining the prognosis for long term survival in a subject having an autoimmune disorder, the kit comprising reagents for assessing expression of the markers of the invention. Preferably, the reagents may be an antibody or fragment thereof, wherein the antibody or fragment thereof specifically binds with a protein corresponding to a marker from Table I. For example, antibodies of interest may be commercially available, or may be prepared by methods known in the art. For example, in one embodiment the antibody used is an anti-mouse GITR/TNFRSF18 polyclonal antibody #AF524 (Goat IgG) available from R&D Systems (Minneapolis, Minn.). Optionally, the kits may comprise a polynucleotide probe wherein the probe specifically binds with a transcribed polynucleotide corresponding to a CD25⁺ differential marker selected from the group consisting of the markers listed in Table I.

The invention further provides kits for assessing the suitability of each of a plurality of compounds for inhibiting an autoimmune disorder or cancer in a subject. Such kits include a plurality of compounds to be tested, and a reagent (i.e., antibody specific to corresponding proteins, or a probe or primer specific to corresponding polynucleotides) for assessing expression of a CD25⁺ differential marker listed in Table I.

Computer Readable Means and Arrays

Computer readable media comprising CD25⁺ differential marker(s) of the present invention is also provided. As used herein, “computer readable media” includes a medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable mediums can be used to create a manufacture comprising computer readable medium having recorded thereon a marker of the present invention.

As used herein, “recorded” includes a process for storing information on computer readable medium. Those skilled in the art can readily adopt any of the presently known methods for recording information on computer readable medium to generate manufactures comprising the markers of the present invention.

A variety of data processor programs and formats can be used to store the marker information of the present invention on computer readable medium. For example, the polynucleotide sequence corresponding to the markers can be represented in a word processing text file, formatted in commercially-available software such as WordPerfect and Microsoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the markers of the present invention.

By providing the markers of the invention in computer readable form, one can routinely access the CD25⁺ differential marker sequence information for a variety of purposes. For example, one skilled in the art can use the nucleotide or amino acid sequences of the invention in computer readable form to compare a target sequence or target structural motif with the sequence information stored within the data storage means. Search means are used to identify fragments or regions of the sequences of the invention which match a particular target sequence or target motif.

Biochips and Arrays

The invention also includes an array comprising a panel of markers of the present invention. The array can be used to assay expression of one or more genes in the array.

It will be appreciated by one skilled in the art that the panels of CD25⁺ differential markers of the invention may conveniently be provided on solid supports, as a biochip. For example, polynucleotides may be coupled to an array (e.g., a biochip using GeneChip® for hybridization analysis), to a resin (e.g., a resin which can be packed into a column for column chromatography), or a matrix (e.g., a nitrocellulose matrix for northern blot analysis). The immobilization of molecules complementary to the marker(s), either covalently or noncovalently, permits a discrete analysis of the presence or activity of each marker in a sample. In an array, for example, polynucleotides complementary to each member of a panel of markers may individually be attached to different, known locations on the array. The array may be hybridized with, for example, polynucleotides extracted from a blood sample from a subject. The hybridization of polynucleotides from the sample with the array at any location on the array can be detected, and thus the presence or quantity of the marker in the sample can be ascertained. In a preferred embodiment, an array based on a biochip is employed. Similarly, Western analyses may be performed on immobilized antibodies specific for different polypeptide markers hybridized to a protein sample from a subject.

It will also be apparent to one skilled in the art that the entire marker protein or polynucleotide molecule need not be conjugated to the biochip support; a portion of the marker or sufficient length for detection purposes (i.e., for hybridization), for example a portion of the marker which is 7, 10, 15, 20, 25, 30,35, 40, 45, 50, 55, 60, 65, 70, 75, 100 or more nucleotides or amino acids in length may be sufficient for detection purposes.

In one embodiment, the array can be used to assay gene expression in a tissue to ascertain tissue specificity of genes in the array. In this manner, up to about 12,000 genes can be simultaneously assayed for expression. This allows an expression profile to be developed showing a battery of genes specifically expressed in one or more tissues at a given point in time. In one embodiment the invention provides a kit comprising a brochure which comprises at least 5, more preferably 10, more preferably 25 or more CD25⁺ differential markers, and the same CD25⁺ differential markers in computer readable form.

In addition to such qualitative determination, the invention allows the quantitation of gene expression in the biochip. Thus, not only tissue specificity, but also the level of expression of a battery of markers in the tissue is ascertainable. Thus, markers can be grouped on the basis of their tissue expression per se and level of expression in that tissue. As used herein, a “normal level of expression” refers to the level of expression of a gene provided in a control sample, typically the control is taken from either a CD25⁺ T cell or from a subject who has not suffered from an autoimmune disorder. The determination of normal levels of expression is useful, for example, in ascertaining the relationship of gene expression between or among tissues. Thus, one tissue or cell type can be perturbed and the effect on gene expression in a second tissue or cell type can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the arrays can be used to monitor the time course of expression of one or more genes in the array. This can occur in various biological contexts, as disclosed herein, for example development and differentiation, disease progression, in vitro processes, such as cellular transformation and activation.

The array is also useful for ascertaining the effect of the expression of a gene on the expression of other genes in the same cell or in different cells. This provides, for example, for a selection of alternate molecular targets for therapeutic intervention if the ultimate or downstream target cannot be regulated. In one embodiment of the invention, an array is used to ascertain the effect of the expression of CD25 on the expression of other genes in CD25⁺ T cells or CD25⁻ T cells.

Importantly the invention provides arrays useful for ascertaining differential expression patterns of one or more genes in CD25⁺ versus CD25⁻ T cells. This provides a battery of genes that serve as a molecular target for diagnosis or therapeutic intervention. In particular, biochips can be made comprising arrays not only of the differentially expressed markers listed in Table I, but of markers specific to subjects suffering from specific manifestations or degrees of the disease (i.e., rheumatoid arthritis; systemic lupus erythematosis; psoriasis; multiple sclerosis; insulin-dependent diabetes mellitus (type I diabetes); inflammatory bowel disease including ulcerative colitis and Crohn's disease (regional enteritis); asthma; or allertic rhinitis).

Modifications to the above-described compositions and methods of the invention, according to standard techniques, will be readily apparent to one skilled in the art and are meant to be encompassed by the invention.

This invention is further illustrated by the following examples which should not be construed as limiting. The contents of all references, patents and published patent applications cited throughout this application, as well as the Figures and Tables are incorporated herein by reference.

### EXAMPLES

### Example 1.0

In order to address the need in the art for identification of molecules involved in acquisition of suppression, and the cell surface molecule or short acting cytokine involved in the effector phase of suppression, DNA microarray technology was used to identify CD25⁺ differential markers of the invention. Applying this technology to the field of immunology proved advantageous since it allowed gene expression in well-defined immune cell types. Further, this technology is very powerful in that it allows a systematic analysis of gene expression differences between cell groups with a single hybridization.

DNA microarray technology was used to analyze unique patterns of genes expressed by CD4⁺CD25⁺ T cells. The examples herein below provide 1) the identification of genes uniquely expressed by resting CD4⁺CD25⁺ T cells, 2) analysis of differential gene expression at two time points following TCR activation to search for molecules (cell surface, secreted or internal to the cell), and 3) determination of genes expressed in the resting or activated state of CD4⁺CD25⁺ cells.

In order to effectuate such a development, a system to preactivate CD4⁺CD25⁺ T cells was developed which resulted in the generation of a suppressive bioactivity which was both T cell receptor non-specific and stable for several weeks. Stimulation of CD4⁺CD25⁺ T cells for as little as 3 days with plate-bound anti-CD3 and IL-2 was found to be sufficient to confer the phenotype. As set forth below, gene expression was compared between CD4⁺CD25⁻ and CD4⁺CD25⁺ T cells at three time points (0, 12 and 48 hours) on plate-bound cells using anti-CD3 and IL-2 activation.

As set forth below, results indicated that number of genes (˜32) were differentially expressed between the resting CD25⁻ and CD25⁺ T cells and that a larger number (˜75) were differentially expressed following activation.

### Example 1.1

Mice, Antibodies and Reagents

BALB/c mice (6-8 week old females) were purchased from NCI Frederick animal facility. B10.D2 expressing a transgenic TCR specific for HA (100-120) (HA Tg) (see e.g., Degermann, D. S. et al, On the various manifestations of spontaneous autoimmune diabetes in rodent models. Eur. J. Immunol. 24:3155-60 (1994)). were purchased from the NIAID/Taconic Contract. All mice were housed in SPF conditions. PE labeled anti-CD25 (clone PC61), FITC labeled anti-CD25 (clone 7D4), FITC labeled anti-CD8a (clone 53-6.7), purified anti-CD28 (clone 37.51), FITC labeled anti-TSA-1 (Sca-2, Ly-6E, clone MTS35), purified and biotinylated anti-CD103 (integrin aIEL, clone M290), purified anti-CD3e (clone 145-2C11), and purified and PE labeled anti-CD152 (CTLA-4, clone UC10-4F10-11) purified anti-CD134 (OX40, clone OX-86) purified anti-CDw137 (4-1BB, clone 1AH2), purified anti-CD2 (LFA-2, clone RM2-5) and SA-FITC were purchased from PharMingen (San Diego, Calif.). Purified and FITC labeled anti-CD134 (clone OX-86) were purchased from Serotec (Oxford, United Kingdom). Tri-Color labeled anti-CD4 (clone CT-CD4) was purchased from Caltag. Normal goat IgG, purified anti-GITR, purified anti-IL-17 (clone 50104.11) and anti-IL-17R were purchased from R & D Systems (Minneapolis, Minn.). FITC labeled Donkey anti-Goat was purchased from Jackson ImmunoResearch Laboratories (West Grove, Pa.) Anti-CD8 and anti-PE magnetic beads were purchased from Miltenyi (Auburn, Calif.).

### Example 1.2

T Cell Purification, Stimulation and RNA Isolation

Peripheral lymph nodes (axillary, inguinal, salivary and mesenteric) were harvested from 6-8 week old female BALB/c or HA Tg mice. For most experiments, cells were isolated by magnetic bead separation. Alternatively, cell sorting techniques known in the art were used. Briefly, red blood cells were removed by ACK lysis (Biofluids, Biosource International) and T cells were purified using T cell enrichment columns (R&D Systems). The T cells were depleted of CD8 by incubation with anti-CD8 microbeads followed by sensitive depletion on AutoMACS (Miltenyi) following manufacturer's instructions. CD8⁻ T cells were incubated with PE (phycoerythrin) labeled anti-CD25 for 20 minutes, washed and incubated with anti-PE microbeads for 15 min and purified by double positive selection on AutoMACS. Purity was confirmed by flow cytometry. CD4⁺CD25⁻ and CD4⁺CD25⁺ cells were greater than 98% and 96% respectively, with no CD8⁺ contamination. FACS was also used for purification of cells. Lymph node cells were subject to density sedimentation over Lympholyte M (CederLane) and subsequently incubated with appropriate amounts of TC labeled anti-CD4, PE labeled anti-CD25, and for some applications biotinylated anti-CD103/SA-FITC for 30 minutes. CD4⁺CD25⁺, CD4⁺CD25⁺CD103⁻ and CD4⁺CD25⁺CD103⁺ were separated using BD FACSVantage Turbo sorter.

For cell stimulations, purified cells were cultured in complete RPMI supplemented with 100 U/ml IL-2 at 37° C. for 0, 12 or 48 hrs in 24 well plates precoated with 5 μg/ml anti-CD3. RNA was purified at the various time points using RNeasy columns (Qiagen) according to manufacturer's instructions. Flow cytometry was also performed at the various time points using TC labeled CD4, PE or FITC labeled anti-CD25 in combination with GIgG/FITC labeled donkey anti-Goat, anti-GITR/donkey anti-goat FITC, biotinylated CD103/SA FITC, FITC-labeled anti-TSA-1, FITC labeled OX40 or PE labeled CTLA-4.

### Example 1.3

DNA Microarray Hybridization and Analysis

RNA isolation and chip analysis was performed as follows. Total RNA was isolated from cell cultures using the Qiagen RNeasy kit. Ten μg of total RNA was quantitatively amplified and biotin-labeled according to Byrne et al. Briefly, RNA was converted to double-stranded cDNA using an oligo dT primer that has a T7 RNA polymerase site on the 5′ end [5′-GGCCAGTGAATT GTAATACGACTCACTATAGGGAGGCGG-(T₂₄)-3′]. The cDNA was then used directly in an in vitro transcription reaction in the presence of biotinylated nucleotides Bio-11-UTP and Bio-11-CTP (Enzo, Farmingdale, N.Y.). To improve hybridization kinetics, the labeled antisense RNA was fragmented by incubating at 94° C. for 35 minutes in 30 mM MgOAc, 100 mM KOAc. Hybridization to Genechips (Affymetrix, San Jose, Calif.) displaying probes for 11,000 mouse genes/ESTs was performed at 40° C. overnight in a mix that included 10 mg fragmented RNA, 6×SSPE, 0.005% Triton X-100 and 100 mg/ml herring sperm DNA in a total volume of 200 ml. Chips were washed, stained with phycoerythrin-streptavidin and read using an Affymetrix Genechip scanner and accompanying gene expression software. Labeled bacterial RNAs of known concentration were spiked into each chip hybridization mix to generate an internal standard curve, allowing normalization between chips and conversion of raw hybridization intensity values to mRNA frequency (mRNA molecules per million). See generally, Byrne M C, Whitley M Z, Follettie M T, 2000, Preparation of mRNA for expression monitoring, In Current Protocols in Molecular Biology 22.2.1-22.2.13. John Wiley and Sons, Inc. (New York).

### Example 1.4

In Vitro Proliferation Assays

Suppression assays were performed as previously described. Briefly, CD4⁺CD25⁻ (5×10⁴) cells were cocultured with irraditaed T-depleted splenocytes (5×10⁴) in the presence of 0.5 μg/ml anti-CD3 or 10 μM HA(110-120). Into the cultures either anti-GITR, anti-CD103, anti-CTLA-4, anti-SCA-2, anti-OX-40, anti-4-1BB, anti-IL-17, anti-IL-17R or control Ig was added. To all cultures, titrated numbers of CD4⁺CD25⁺ cells were added to final responder cell:suppressor cell ratios of 1:0, 2:1, 4:1, 8:1, 16:1 and 32:1. Cultures were then pulsed with 1 μCi of 3H-thymidine for the final 6-12 hour of a 65-72 hour culture.

For some assays, BALB/c CD4⁺CD25⁺ T cells were prestimulated for a minimum of 3 days with either 5 μg/ml plate-bound anti-CD3 or 0.5 μg/ml soluble anti-CD3 in the presence of 100 U/ml rIL-2. These activated CD4⁺CD25⁺ cells were used as described in the suppression assays with either BALB/c CD4⁺CD25⁻ T cells stimulated with 0.5 μg/ml anti-CD3, or HA Tg CD4⁺CD25⁻ T cells stimulated with 10 μM HA (110-120).

Costimulation assays were performed as follows. CD4⁺CD25⁻ T cells (5×10⁴) were cocultured with irradiated T depleted spleen (5×10⁴) in the presence of various doses of soluble anti-CD3. To some wells 2 or 10 μg/ml anti-CD28 or anti-GITR were added. Cultures were pulsed with 1 μCi ³H-thymdine for the last 6-12 hours of 65-72 hour cultures.

### Example 1.5

Results: DNA Microarray Analysis of Resting CD4⁺CD25⁺ and CD4⁺CD25⁻ T cells

In order to identify the molecular markers for the immunoregulatory functions of CD25⁺ T cells, two independent isolations of RNA from CD25⁺ and CD25⁻ T cells were hybridized to genechips monitoring the expression of 11,000 genes and ESTs. Genes which were significantly differentially expressed between the two resting populations in both replicates are shown in FIG. 1. Of the 32 genes identified, 23 had upregulated mRNA levels and 9 downregulated mRNA levels in resting CD25⁺ T cells relative to resting CD25⁻ T cells. A wide variety of functional gene classes were identified, including cell surface receptors, secreted molecules, transcription factors, signaling molecules, small G proteins, and kinases, in addition to a number of uncharacterized ESTs. For example, five cell surface antigens (CTLA-4, Galectin-1, GITR, OX-40, CD103, SCA-2) were differentially expressed in the CD4⁺CD25⁺ T cells, while only one (TCR a-chain) was increased in the CD4⁺CD25⁻. Expression of CTLA-4, a T cell inhibitory receptor, that has previously been reported to be constitutively expressed by CD25⁺ T cells (Takahashi, T.et al, Immunologic self-tolerance maintained by CD25(+)CD4(+) regulatory T cells constitutively expressing cytotoxic lymphocyte-associated antigen 4. J. Exp. Med. 192:303-10 (2000)), was readily detected by the chip analysis as having increased levels of mRNA. Expression of CD25 mRNA in these two experiments was not detected even though this antigen is readily detected on the cell surface of CD4⁺CD25⁺ T cells by flow cytometry. This result most likely reflects the low level of transcription of this gene in resting CD25⁺ T cells as CD25 mRNA could readily be detected following T cell activation (see below). These differences in gene expression may reflect previous activation history, differences in the way these cells interact with their environment and/or unique mechanisms for regulating immunoregulatory activity.

### Example 1.6

Identification of Genes Differentially Expressed in CD4⁺CD25⁺ T Cells upon Activation

The immunoregulatory bioactivity of CD25⁺ T cells is dependent upon stimulation through the T cell receptor. Accordingly, the example herein used activated CD25⁺ and CD25⁻ T cells to identify genes whose products contributed to this functional activity.

A system was developed to preactivate CD4⁺CD25⁺ T cells resulting in the generation of a suppressive bioactivity that is TCR non-specific and stable for several weeks. Stimulation of CD4⁺CD25⁺ T cells for as little as 2 days with plate-bound anti-CD3 in the absence of accessory cells and IL-2 was determinded to be sufficient to confer this phenotype. The gene expression was compared between CD4⁺CD25⁻ and CD4⁺CD25+T cells prior to stimulation and after 12 and 48 hr of anti-CD3 and IL-2 stimulation. Genechip analysis was used to construct a global kinetic activation series after stimulation of CD25+or CD25⁻ T cells.

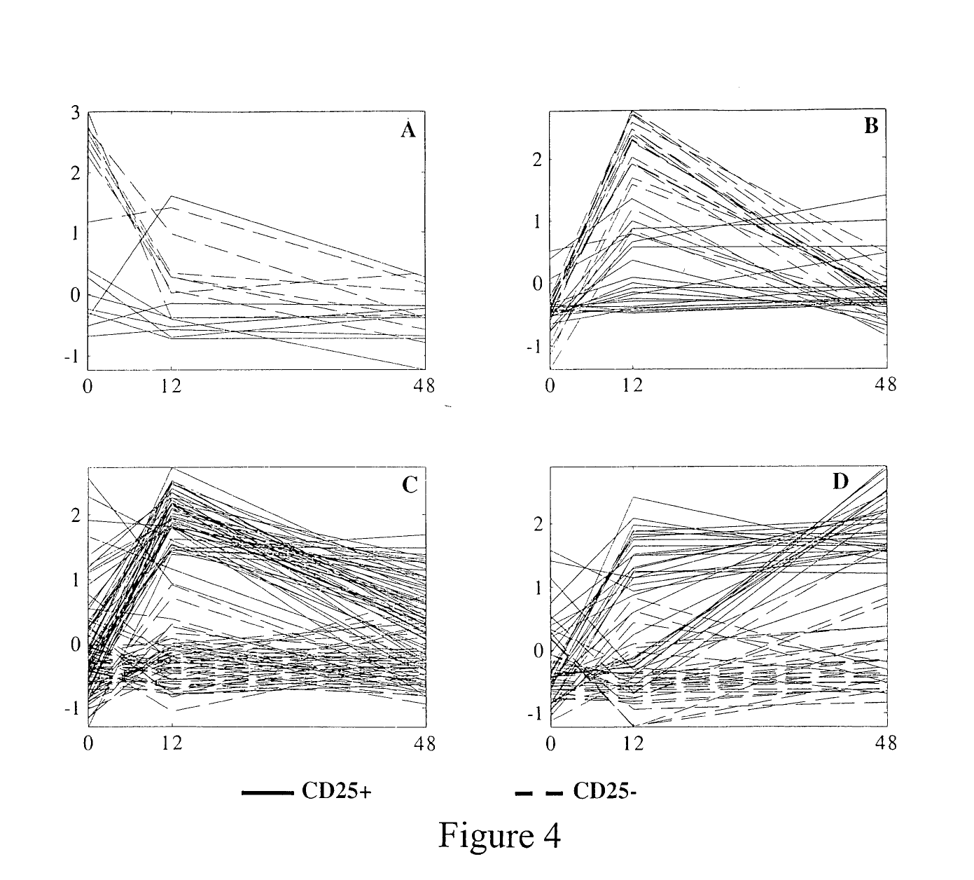

The cellular activation and genechip analysis were performed twice, and only those genes displaying similar profiles in both repetitions were included in the analysis. Over 100 genes were significantly and reproducibly differentially expressed in at least one of the three timepoints. These genes were clustered using the Self-Organizing Map (SOM) algorithm, a statistical method for grouping genes based on expression patterns independent of expression magnitude (See e.g., Tamayo et al, Proc. Natl. Acad. Sci. USA, 1999, 96:2907-12). Results are shown in FIG. 4. This analysis revealed four basic patterns of expression in CD25⁺ T cells relative to CD25⁻ T cells. Cluster A contains markers which are increased in CD25⁻ cells at 0 hours, but for which expression dropped to CD25⁺ levels at the 12 and 48 hour timepoints. Markers in cluster B were induced more strongly in CD25⁻ than CD25⁺ T cells at 12 hours, but the induction was transient, with expression levels dropping to baseline by 48 hours. This group included molecules characteristic of the productive immune response, including IL-2, lymphotoxin, lymphotactin and JAK-2. The lack of induction of these mRNAs in the CD25⁺ T cells was consistent with an anergic phenotype. Cluster C identified markers displaying the opposite behavior, i.e. induction occurred exclusively in the CD25⁺ population at 12 hours, but was back to baseline by 48 hours. Cluster D contained markers that were increased in the CD25⁺ population at both 12 and 48 hours. These two latter clusters, in which gene expression is heightened in the CD25⁺ population, represented approximately ¾ of the differentially expressed genes. Thus, although these cells display an anergic phenotype, they were competent to respond to stimulation by induction of a large number of genes. The genes populating these four clusters are listed by functional class in Table I.

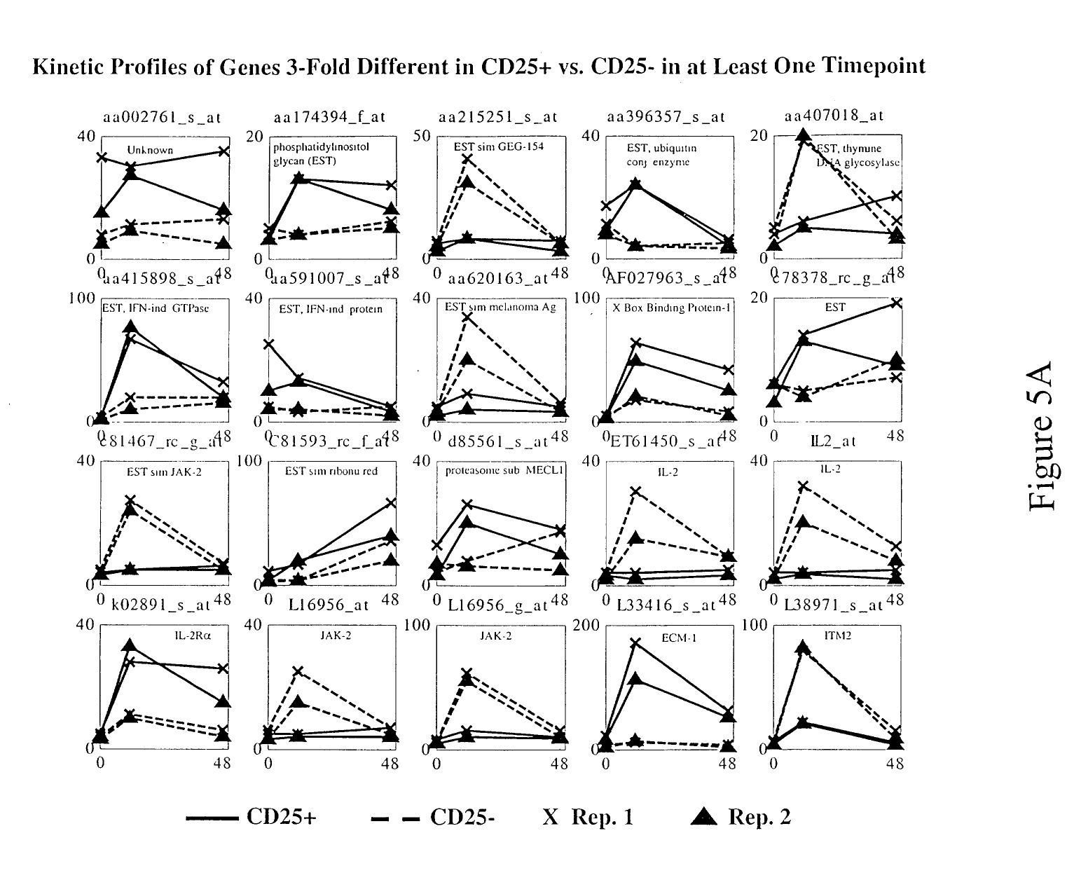

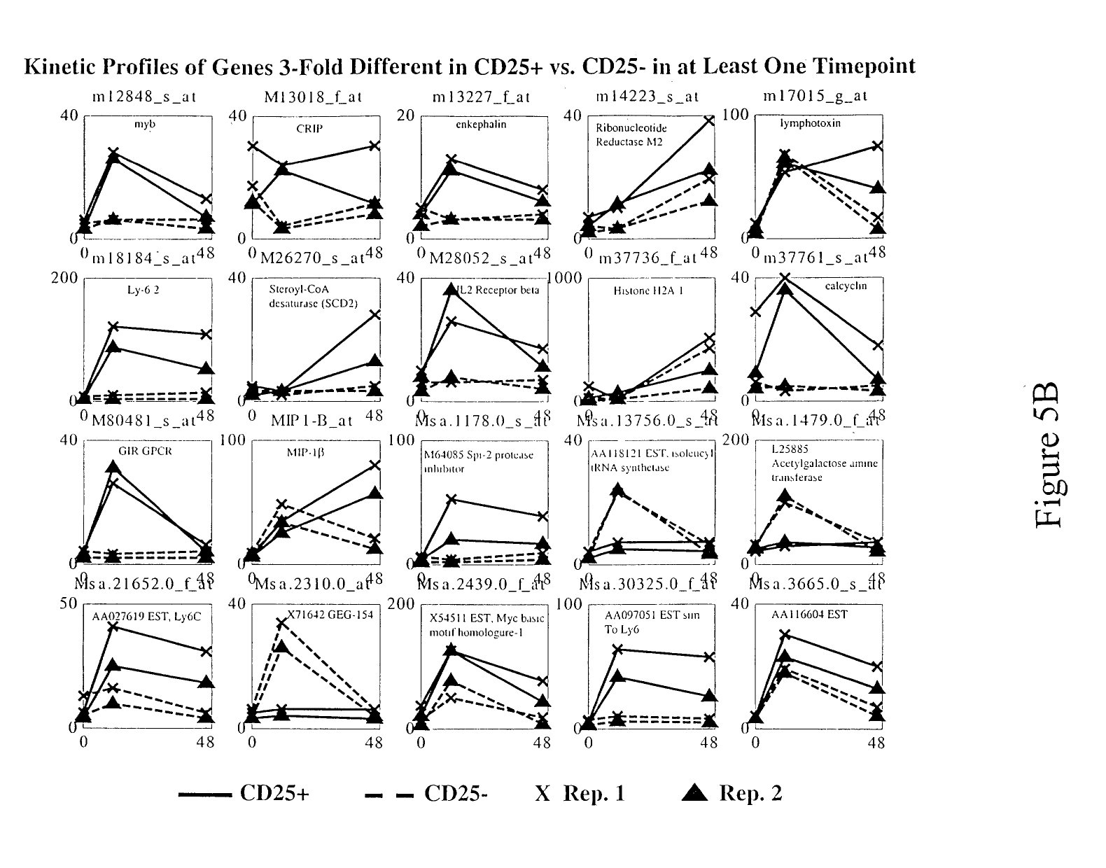

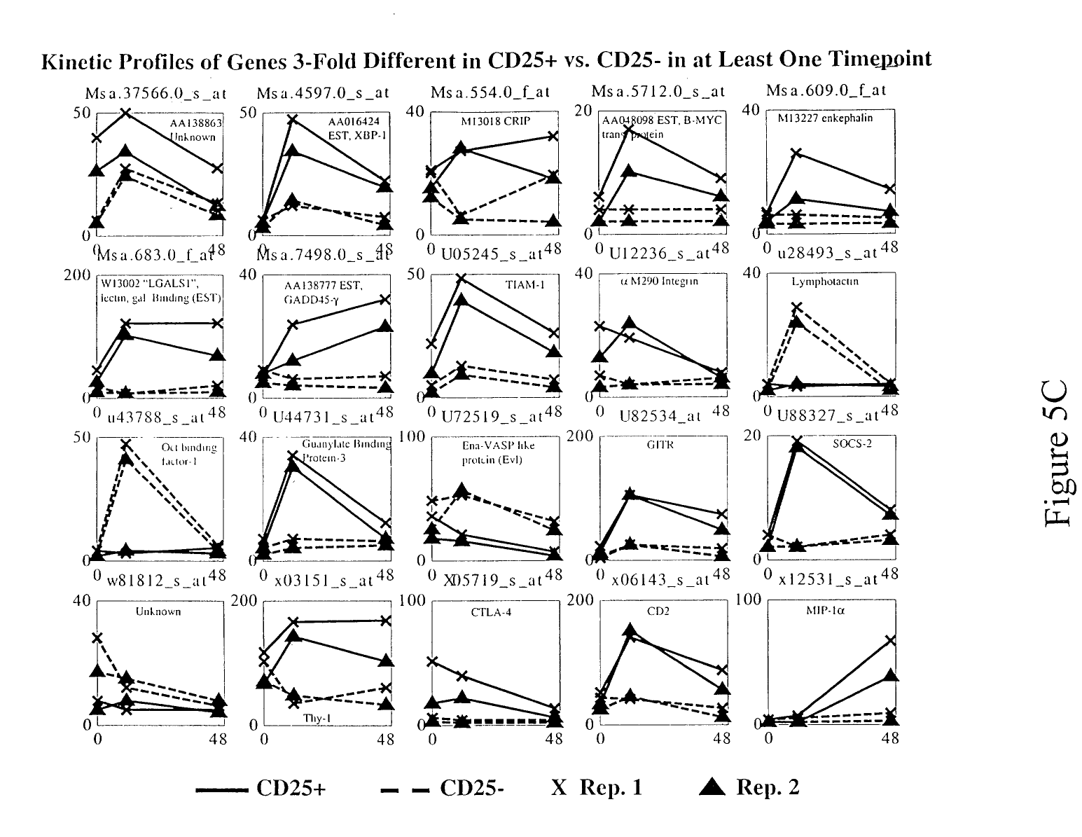

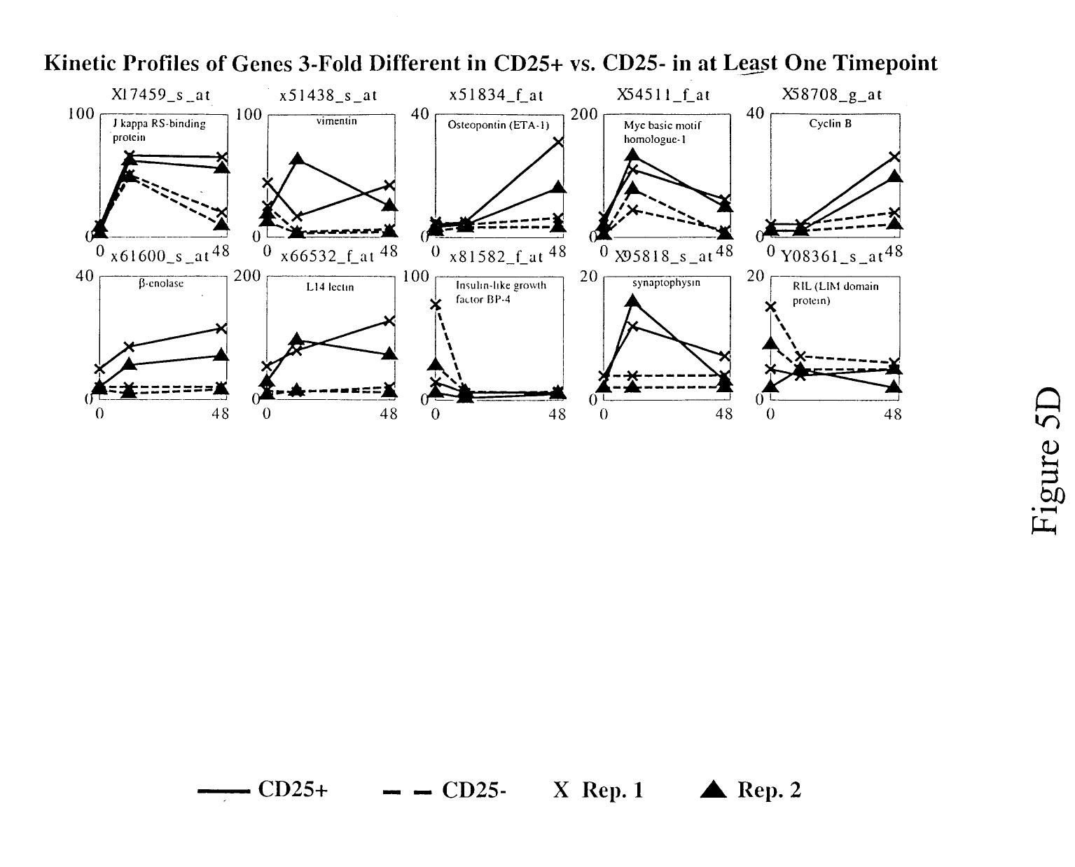

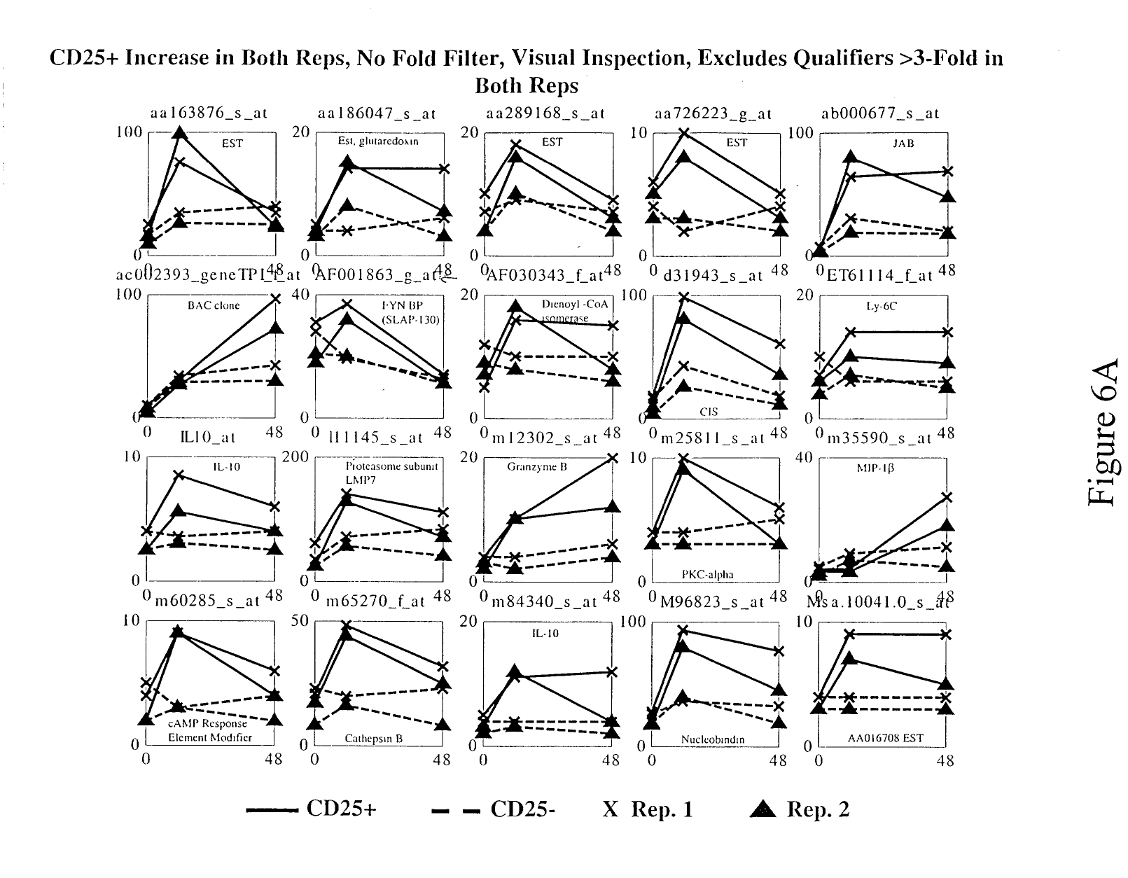

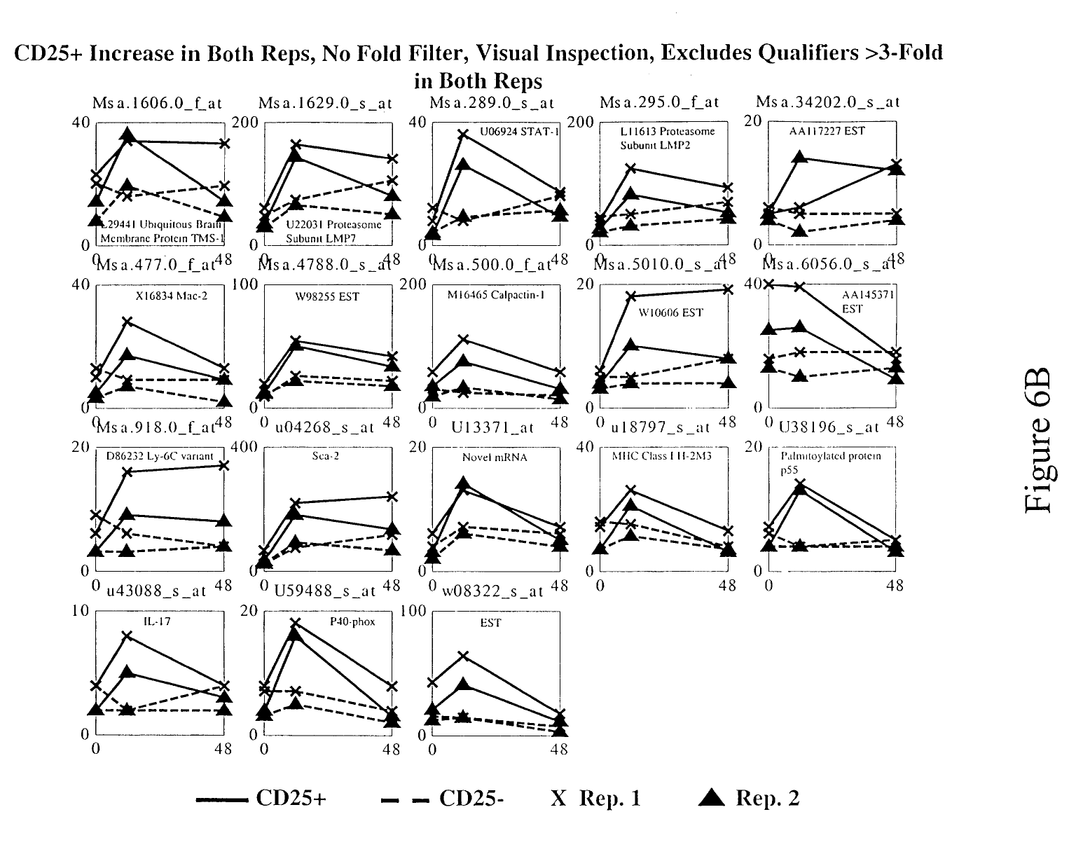

In addition, the expression results for each of these genes was plotted out individually, with each graph presenting the results from both of two replicate experiments (FIGS. 5A-D and 6A-B). FIG. 5 includes those genes which were modulated at least 3-fold in at least one timepoint in CD25⁺ cells relative to CD25⁻ cells, for both replicates. FIG. 6 includes those genes that did not meet the 3-fold criterion, but that were significantly and reproducibly modulated in CD25⁺ cells relative to CD25⁻ cells.

CD4⁺CD25⁺ T cells have certain characteristic of memory/activated T cells particularly the preferential expression of the CD45RB^{low} phenotype. The propensity of the CD25⁺ population to upregulate so many genes upon activation through the T cell receptor indicated a previously activated/memory phenotype. In addition to IL-2Ra (CD25), IL-2Rb and CTLA-4, the activation markers CD2 and OX-40 were found to be preferentially induced in the CD25⁺ population. In addition, the screen reproducibly identified upregulation of GIR (Glucocorticoid Induced Receptor), a G Protein Coupled Receptor whose ligand is unknown, and GITR (Glucocorticoid Induced TNF Receptor), a TNF receptor that when engaged by its ligand (GITR-L), causes activation of the NF-kβ pathway and protection from apoptosis. See Nocentini et al, Proc. Natl. Acad. Sci. USA 94:6216-21 (1997); Kwon et al. J. Biol. Chem. 274:6056-61 (1999); Gurney et al, Curr. Biol. 9:215-8 (1999); Riccardi et al. Cell Death Differ. 6:1182-9 (1999).

A number of mRNAs for secreted molecules were induced preferentially in the CD25⁺ population, including the chemokines Mip-1α and Mip-1β. Theses are involved in recruitment of other cells to sites of immune activation and have been reported to be expressed in anergic cells. The inflammatory protein IL-17; and the immunosuppressive cytokine IL-10 were also identified. Also identified was increased induction of Early T cell Activation-1 (ETA-1), a cytokine that has been reported to regulate the expression of IL-12 and IL-10 in macrophages. Another marker identified included mRNA for Extracellular Matrix Protein-1 (ECM-1), an 85 kd secreted protein which has recently been reported to possess angiogenic activity (see Han et al, FASEB J. 15:988-94 (2001)) and which has not previously been reported in immune cells, was consistently induced in CD25⁺ T cells.

Activated CD25⁺ cells also expressed high levels of mRNA for SOCS-1(JAB) and SOCS-2, two factors that play important roles in down-regulation of cytokine production and cytokine mediated activation. The expression of these proteins may account, in part, for the failure of the CD25⁺ cells to produce IL-2.

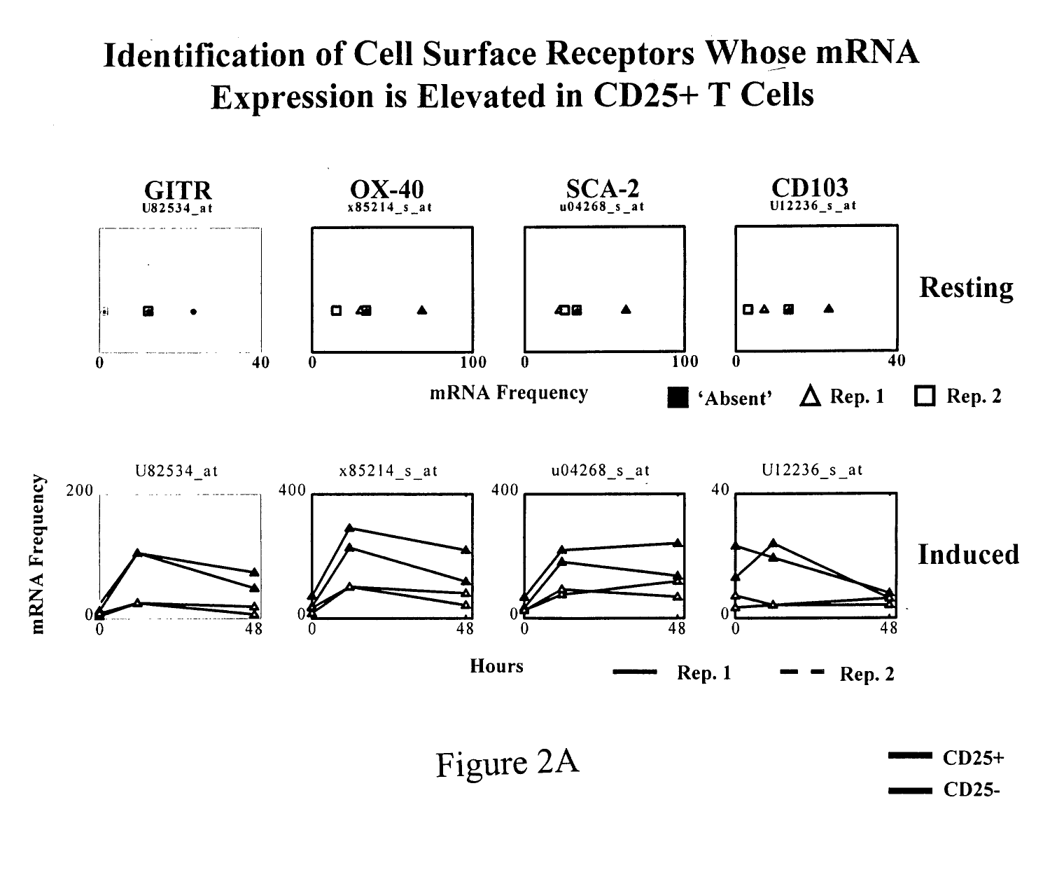

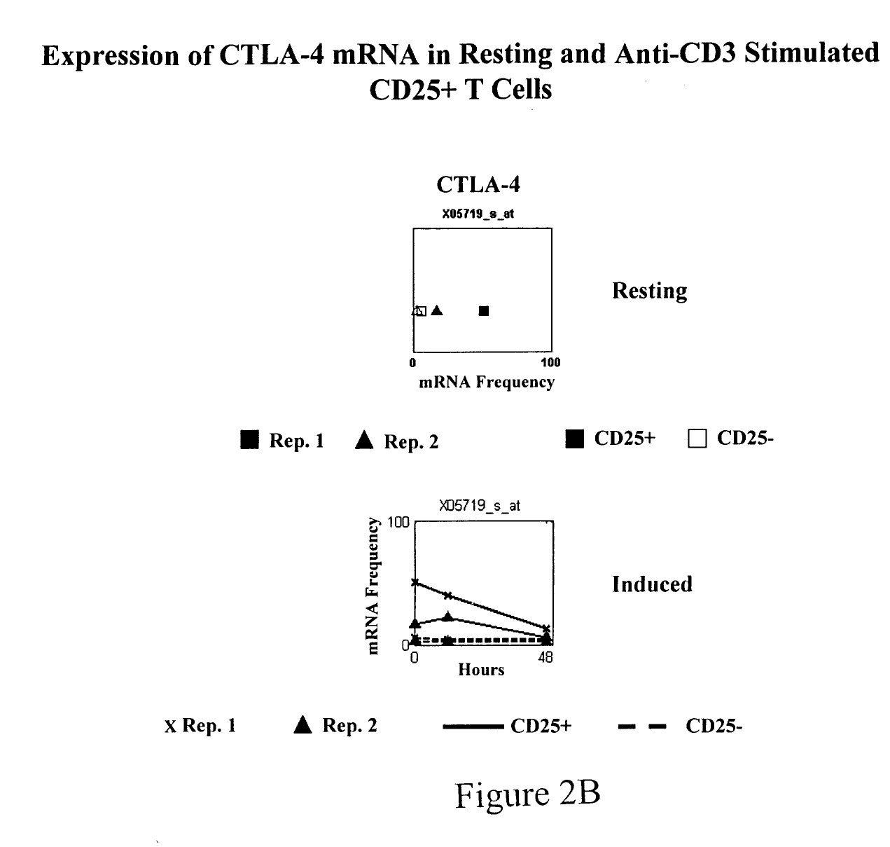

In order to identify molecules that controlled the immunoregulatory activity of CD25⁺ T cells, a focus was placed on cell surface receptors for confirmation of differential expression at the protein level and for initial functional analysis. FIGS. 2A and 2B displays RNA expression resulting from both resting and activated cells for the receptors GITR, OX-40, SCA-2, CD103 and CTLA-4. mRNA for each of these genes was detected as increased in resting CD25⁺ relative to CD25⁻ cells. After activation, expression of GITR, OX-40 and SCA-2 was further induced. In contrast, CTLA-4 mRNA showed no induction at the timepoints monitored, and CD103 did not show consistent upregulation.

### Example 1.7

Differential Expression of Cell Surface Markers

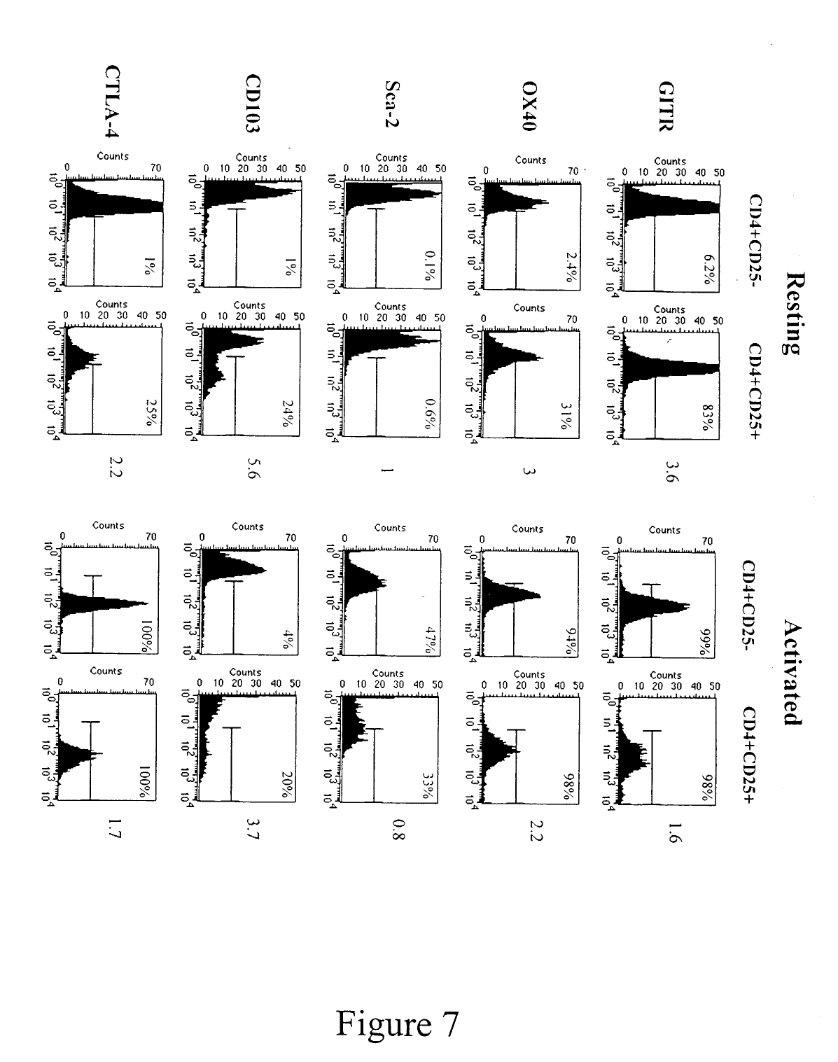

Differential mRNA expression for cell surface molecules was extended to the protein level using flow cytometry. Comparison of CD4⁺CD25⁻ and CD4⁺CD25⁺ cells showed that the molecules GITR, OX40, and CTLA-4 were expressed at a higher level on resting CD25⁺ cells, see FIG. 7. (CD25⁺/CD25⁻ Mean Fluorescence Index (MFI) ratio 3.6, 3, and 2.2, respectively). CTLA-4 was found exclusively in the CD4⁺CD25⁺ population of T cells. TNF Receptor Superfamily members GITR and OX40 were also confirmed to be exclusively expressed on resting CD25⁺ cells. CD103 was found to be expressed on only 20 to 30% of CD25⁺ cells, and was not found on CD25⁻ cells. Although mRNA for SCA-2 was found to be differentially expressed in the CD25⁺ population of cells, there was no detectable surface expression of this molecule on the cell surface of either population. Molecules reported to be expressed on activated T cells (GITR, OX40, SCA-2, and CTLA-4) were upregulated on both cell populations after 48 hr of stimulation with plate-bound anti-CD3 and IL-2. However, the levels of GITR, OX40, and CTLA-4 were increased on CD25⁺ cells, even after activation (CD25⁺/CD25⁻ MFI ratio, 1.6, 2.2, and 1.7 respectively). Interestingly, the expression of CD103, an integrin expressed on all Intraepithelial Lymphocytes (IELs) was not significantly upregulated after activation for 48 hours, and the percentage of CD25⁺ cells that expressed CD103 in the resting and activated state were comparable.

### Example 1.8

Separation of CD25⁺ Cells into CD103⁺ and CD103⁻

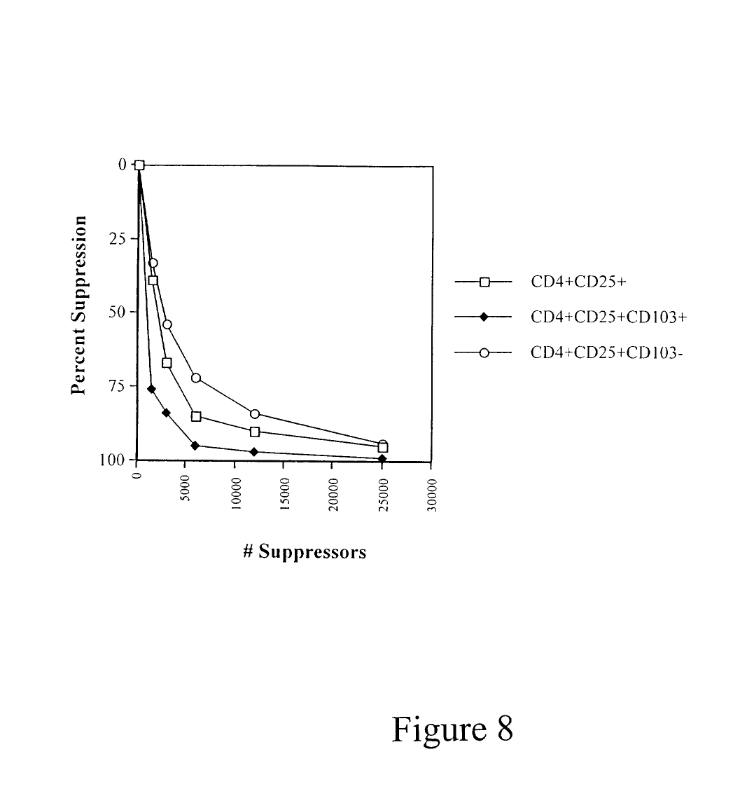

In order to identify additional CD25⁺ differential markers at the protein level, which may be useful in identifying, isolating or manipulating CD25⁺ T cells displaying suppressive bioreactivity, flow cytometry was performed to separate CD25⁺CD103⁺ cells and CD25⁺CD103⁻ T cells. The Bimodal distribution of CD103 on the CD25⁺ T cell population is shown in FIG. 7. Both cell populations were assayed for suppressive bioactivity, and results are shown in FIG. 8. Both CD103⁺CD25⁺ and CD103⁻CD25⁺ were able to suppress anti-CD3 induced proliferation of CD4⁺CD25⁻ T cells.

Consistently, however, the CD103⁺CD25⁺ cells were more efficient, on a per cell basis, at suppressing the proliferation of the responders. In addition, cells expressing CD4⁺CD 103+, without selection for CD25⁺, were able to suppress in vitro proliferation.. Analysis of CD103⁺CD25⁺ cells revealed that they have a phenotype of recently activated cells, showing higher levels of CD69 and lower levels of CD45RB and CD62L by flow cytometry than CD25⁺CD103⁻ T cells.

Thus, the expression of CD103 may define a subpopulation of CD4⁺CD25⁺ cells that have been recently activated in vivo, thereby acquiring a heightened suppressive phenotype. Further, CD25 remains an excellent marker for the suppressor cells, as the regulatory phenotype was not segregated among other subpopulations.

### Example 1.9

Reversal of Suppression with Anti-GITR Antibody

Analysis of the differential expression of the DNA microarray identified many candidate genes that may be involved in the suppressive function of the CD4⁺CD25⁺ cells as well as genes involved in the regulation of the suppressive phenotype.

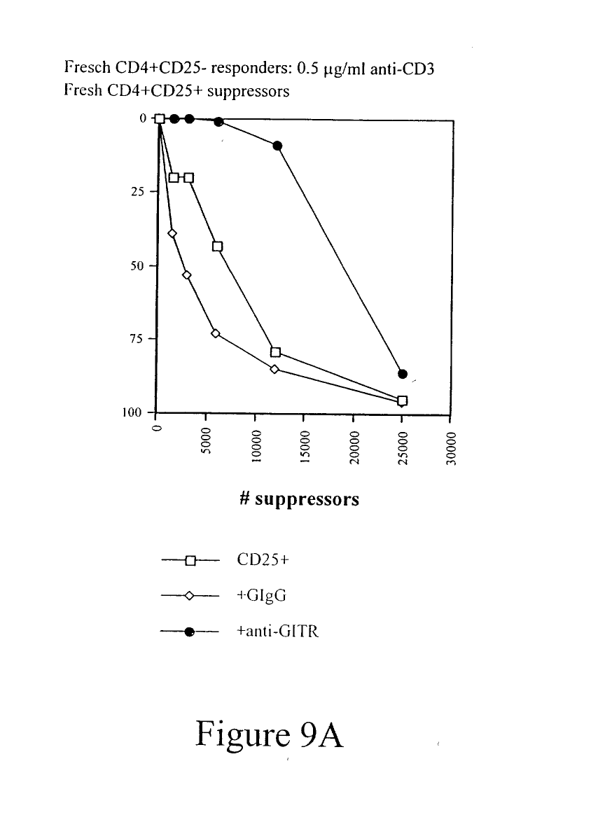

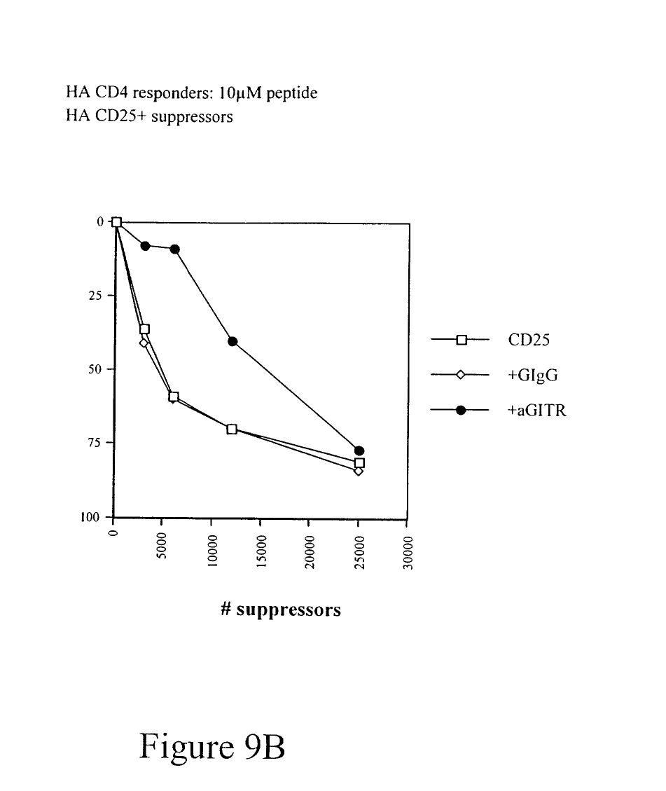

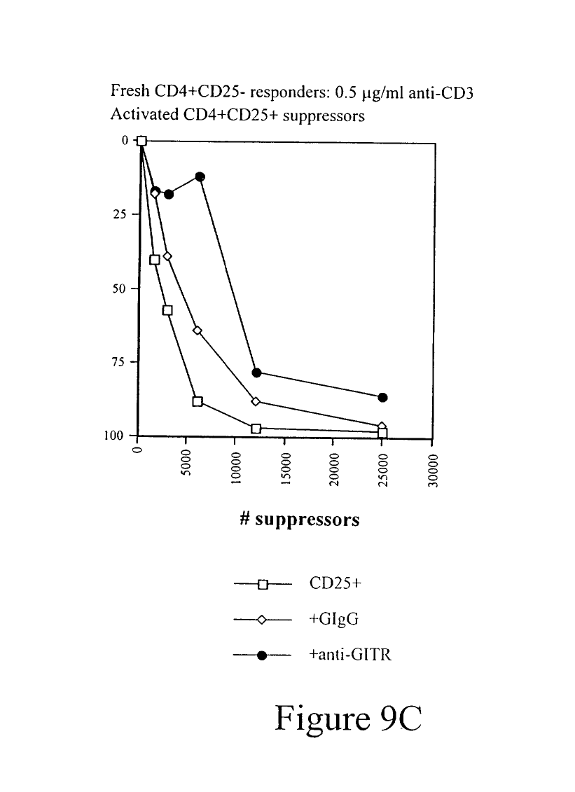

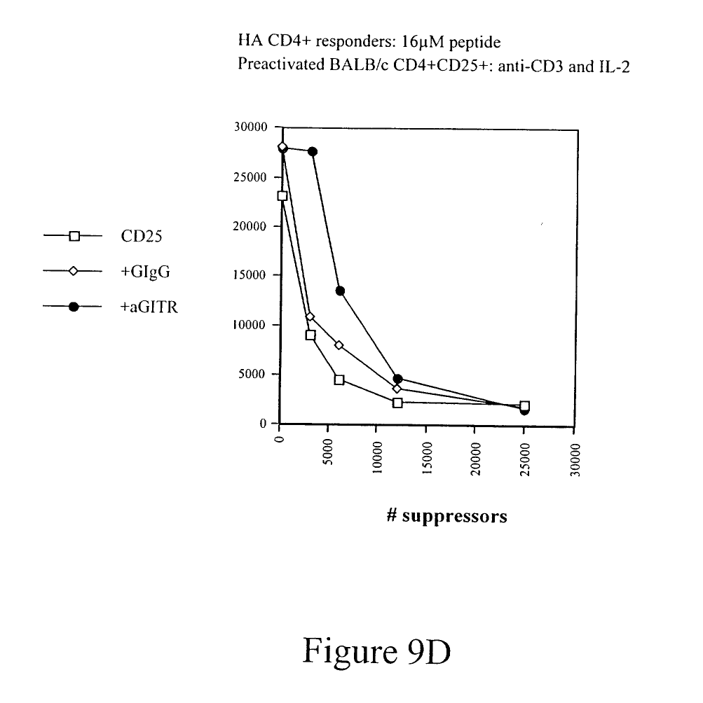

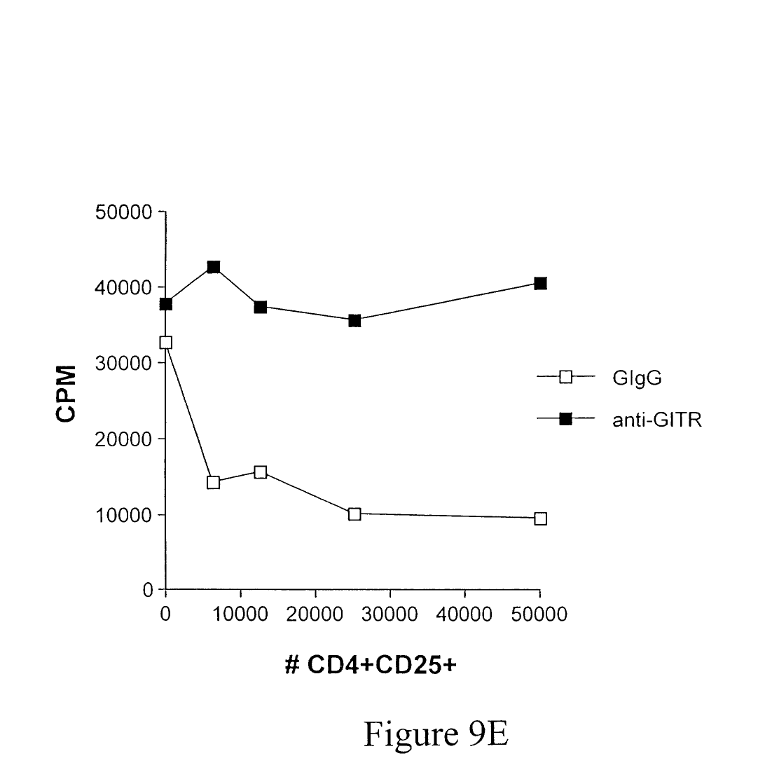

As MAbs or polyclonal antibodies are available to many of the products of the differentially expressed genes, the capacity of these antibodies to reverse suppression in co-cultures of CD4⁺CD25⁺ and CD4⁺CD25⁻ T cells was tested. Antibodies to CD103, CTLA-4, 4-1BB, OX40, CD2, IL-17 and IL-17R had no effect on the ability of the CD4⁺CD25⁺ cells to exert suppression. A polyclonal antiserum to the mouse GITR extracellular domain, in contrast, was able to reverse suppression induced by freshly isolated CD4⁺CD25⁺ cells from normal BALB/c animals in response to anti-CD3 (FIG. 9A). In addition, anti-GITR reversed the capacity of CD4⁺CD25⁺ T cells isolated from HA Tg mice to inhibit the responses of HA Tg CD4⁺CD25⁻ T cells to their specific peptide (FIG. 9B). FIG. 9A illustrates the anti-CD3 response, FIG. 9B illustrates the antigen. In addition, this antibody was effective with preactivated CD4⁺CD25⁺ cells.

The capacity of anti-GITR to reverse suppression by anti-CD3 and IL-2 activated CD25⁺ T cells was evaluated. (See FIG. 9C). Reversal of suppression of the response to anti-CD3 was consistently observed in these studies, but only at low ratios of suppressors to responders. Suppression was not abrogated at higher suppressor to responder cell ratios (e.g., 32:1, 16:1, 8:1). See FIGS. 9C and 9D. FIG. 9C was anti-CD3 and FIG. 9D was antigen.

In order to determine whether anti-GITR would reverse suppression under conditions which preclude restimulation of the activated CD25⁺ cells in the suppression assay, we tested whether anti-GITR would reverse suppression mediated by activated CD25⁺ T cells from normal BALB/c mice of the response of HA-specific T cells to HA (FIG. 9D). Again, at low suppressor to responder ratios, suppression was consistently reversed (See FIG. 9D).

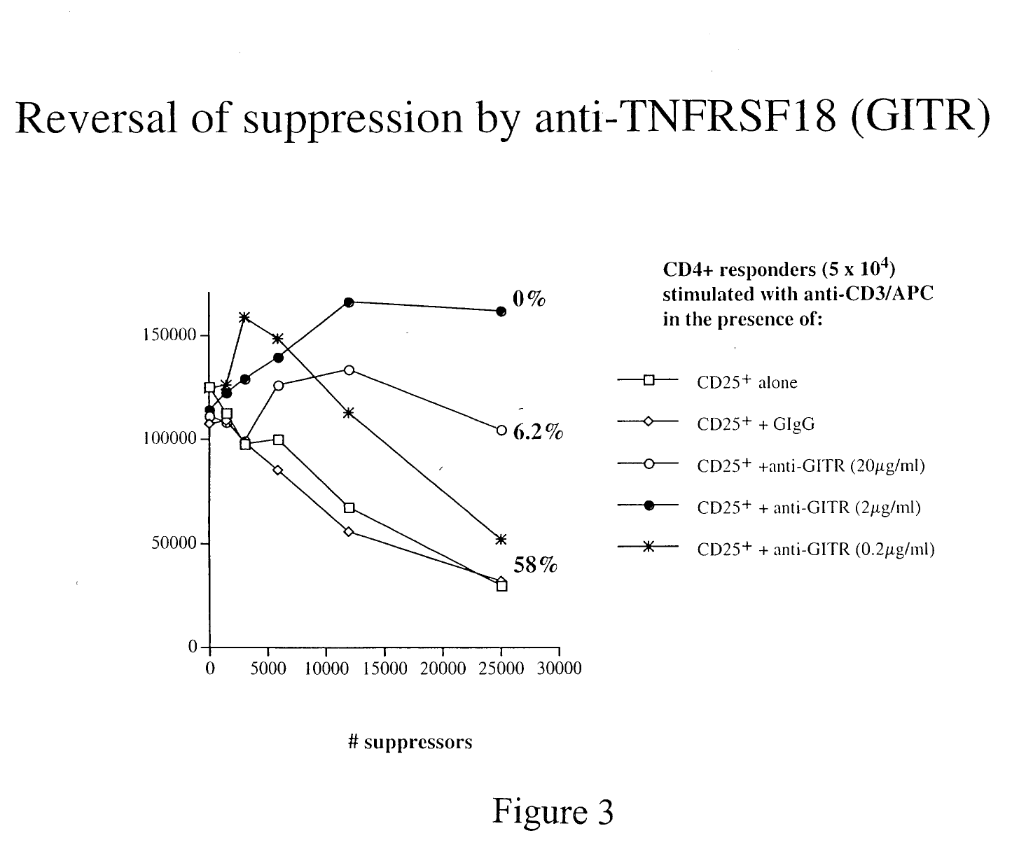

In addition, the degree of suppression abrogation was proportional to the amount of anti-GITR antibody added (see FIG. 3). These results indicated that interaction of GITR on the CD4⁺CD25⁺ with its ligand may be mechanism to suppress in vitro proliferation. Alternatively, this interaction may be required for the induction of the suppressive phenotype in combination with TCR stimulation.

### Example 1.10

CD8 Suppression

As CD25⁺ T cells can also suppress the responses of CD8⁺ T cells, we also tested whether anti-GITR could reverse CD25⁺ mediated suppression of the activation of CD8⁺ cells. Results identical to those seen with CD4⁺ responders were observed, and are set forth in FIG. 9E.

### Example 1.11

Soluble Anti-GITR Does Not Provide a CD28-like Costimulatory Signal to CD4⁺CD25⁻ Responders

Without limitation as to mechanism, the results presented above are consistent with an interaction of GITR on the CD4⁺CD25⁺ cells with its ligand on the responder cells being directly involved in mediating suppression. However, both the gene chip and the flow cytometry studies herein demonstrate that the GITR is also induced on CD25⁻ T cells by T cell activation.

Previous studies have shown that CD4⁺CD25⁺ mediated suppression can be reversed by the addition of IL-2 or anti-CD28 to the co-cultures. These studies demonstrated that the presence of exogenous IL-2 or the induction of endogenous IL-2 production by a strong costimulus circumvented or masked the block in IL-2 production. The addition of anti-CTLA-4 has also been reported to reverse suppression, but this effect has not seen with human CD25⁺ T cells, nor can it be readily reproduced in mouse studies.

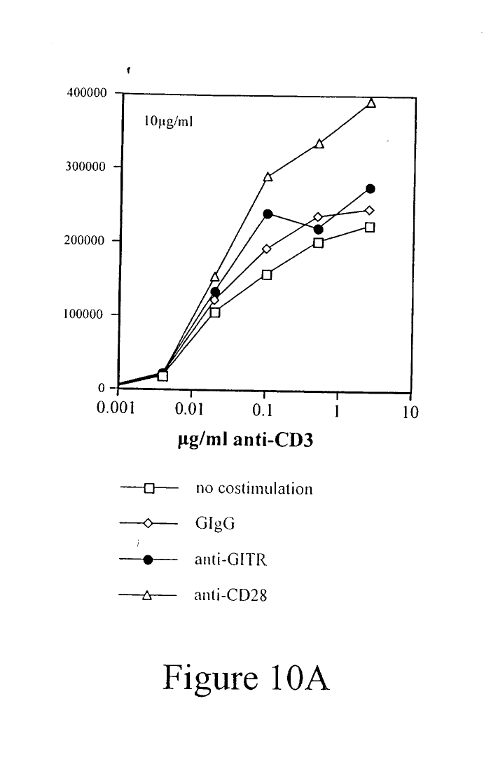

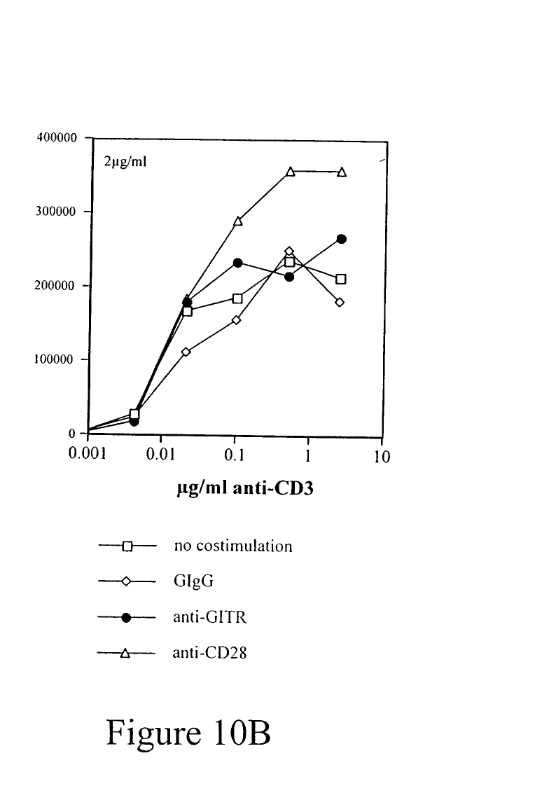

In order to determine if reversal of suppression mediated by anti-GITR was due to its ability to deliver a strong stimulatory signal to the responder T cells, and to determine if anti-GITR was providing a costimulatory signal similar to anti-CD28, CD4⁺CD25⁻ cells were stimulated with graded different concentrations of soluble anti-CD3 in the presence of either anti-CD28 or anti-GITR. While anti-CD28 increased the proliferation of the responders (FIGS. 10A-B) at the concentration of anti-CD3 used in the suppression assays (0.5μg/ml), anti-GITR had a minimal effect. Thus, anti-GITR may not be reversing suppression by stimulating the production of IL-2 by the responder T cells. In addition, treatment of CD4⁺CD25⁻ responders with anti-GITR did not appear to allow protection from apoptosis as a similar percentage of apoptotic cells are found when culturing with anti-CD3 alone or in the presence of control Ig or anti-GITR (20, 22, and 19% respectively).

### Example 1.12

Implications and Discussion

Microarray technology has been used herein to compare gene expression in the resting and activated state of two highly purified subpopulations of the CD4⁺ lineage of lymphocytes that have different functional properties in vivo and in vitro. While CD4⁺CD25⁻ lymphocytes represent cells that can produce IL-2, proliferate in vitro, and differentiate into effector Th1/Th2 cells, CD4⁺CD25⁺ T cells fail to respond to stimulation via the TCR by producing IL-2 and have the unique capacity to suppress the production of IL-2 and other effector cytokines by both CD4⁺ and CD8⁺CD25⁻ T cells. Without limitation as to mechanism, the examples herein support the view that these two populations of CD4⁺ T cells differ in only a small number of the 11,000+ genes and EST's tested, but that many of the observed differences are closely correlated with the distinct functional properties of these subpopulations.

A novel discovery of the invention was the identification of unique genes that encode cell surface antigens exclusively expressed on the CD25⁺ population. Several genes were identified that fit this description including OX-40, SCA-2, CD103 and GITR. The use of antibodies to all of these antigens facilitated a comparison of the mRNA data with protein expression at the cell surface. Although expression of SCA-2 mRNA was detected, expression of SCA-2 could not be detected on resting CD25⁺ T cells, but was easily detectable on both activated CD25⁺ and CD25⁻ cells. On the other hand, the other four antigens (OX-40, GITR, CD103, and GITR) were readily detectable on the cell surface of resting CD25⁺, but not CD25⁻, T cells.

Several groups have previously shown that CD25⁺ T cells were the only T cells in the normal lymphocyte pool that expressed CTLA-4. The identification of the gene encoding this antigen in the microarray validates this new technology.

The expression of CD103 was unique in that it was only expressed on a minor (˜30%) subpopulation of CD25⁺ T cells and the level of expression was not modulated by T cell activation. However, both CD25⁺CD103⁺ and CD25⁻CD103⁺ T cells were capable of inhibiting the activation of CD25⁻ cells in vitro. This result was similar to observations herein with other antigens (CD45, CD62L, CD69, CD38) that appeared to subdivide the CD25⁺ population. Cells which constitutively expressed CD25, consistently inhibited T cell activation in vitro.

The CD25⁺ population was unique in that it fails to respond to activation via the TCR by producing IL-2 and proliferating even in the presence of potent costimulatory signals such as agonistic anti-CD28. It was therefore of interest that three members (CIS, SOCS-1 (also known as JAB) and SOCS-2) of the suppressors of cytokine signaling (SOCS) family appeared to be exclusively expressed by the CD25⁺ cells and to be upregulated during T cell activation. This result by itself is surprising as SOCS protein expression is in most cases not observed in “resting” cells, but is only induced by cytokine signals that activate STAT proteins which in turn bind to SOCS gene promoters. The apparent constitutive expression of members of the SOCS family in the CD25⁺ population was most likely secondary to their activation state in vivo.

As IL-2 is required in vitro for the survival/maintenance of the CD25⁺ population, the SOCS proteins may be induced in response to this cytokine or other cytokines needed for the homeostatic control of these cells. Increased levels of SOCS expression may be required to carefully control the size of the CD25⁺ population in vivo to achieve a fine balance between the need to suppress autoreactivity and the danger of suppressing responses to foreign antigens. SOCS-1 is a negative regulator of STAT5 activation and thereby may play a role in regulating responses to IL-2, IL-3, and erythropoietin. SOCS-1 has been shown to bind to all four JAK kinases and SOCS-1 −/− mice have a phenotype characterized by enhanced responsivity to IFN-g. As large amounts of IFN-g may be produced during an immune response to an infectious agent, the capacity of the CD25⁺ T cells to preferentially upregulate this inhibitor may diminish their suppressive function during protective immune responses.

Preliminary semiquantitative RT-PCR studies have demonstrated that the SOCS-2 was expressed at high levels in the CD25⁺ cells and that these levels were increased after activation; in contrast, only low levels of SOCS-2 were detected in the CD25− cells even after activation. Studies with SOCS-2 −/− mice have suggested a role for this inhibitor in the regulation of the responses to insulin-like growth factor-1 (IGF-1) and growth hormone. The selective expression of this inhibitor in the CD4⁺CD25⁺ population of suppressor T cells is clearly worthy of more detained study.

Activation of the CD25⁺ T cells may result in induction of a cell surface molecule that mediates their suppressive effects by binding to a ligand on the responder CD25⁻ T cells. The ligand for the purported suppressor effector molecule may be constitutively expressed, but might also be induced during the T cell activation process.

Such an effector molecule may be identified by analyzing the genes induced following activation of the CD25⁺ cells. For example, such a gene may be represented in the population of ESTs that appeared to be selectively induced on the CD25⁺ T cells (Table I).

As part of our screening procedures to identify functionally important molecules on the CD25⁺ population, antibodies were obtained to both cytokines and cell surface antigens that were either selectively expressed on resting CD25⁺ T cells or induced during activation. A polyclonal antiserum to the GITR reversed CD25⁻ mediated T cell suppression indicating that the GITR functioned in the suppression process.

Without limitation as to mechanism, there are a number of roles GITR may potentiate in mediating suppression. The first is that GITR may represents a suppressor effector molecule. Indeed, certain other members of the TNFSF have been shown to “reverse signal” by binding to their ligands. Thus, engagement of Fas-L by FAS resulted in growth arrest and eventual apoptosis of Fas-L expressing lymphocytes, indicating that Fas-L was transducing signals. See e.g., Desbarats, J. et al, Nature Medicine 4:1377-82 (1998). The phenotype of suppression seen following engagement of Fas-L closely resembles that seen during CD25⁻ mediated suppression, i.e., inhibition of IL-2 production, cell cycle arrest, and eventually apoptosis of the responders. However, GITR is constitutively expressed on resting CD25⁺ T cells and resting CD25⁺ cells are incapable of mediating suppression. Moreover, the capacity of saturating concentrations of the anti-GITR to modestly inhibit suppression induced by activated CD25⁺ cells suggests that GITR may not be directly delivering the suppressive signal.

A comparison of the structure of GITR with other members of the TNFRSF raises the possibility that GITR may play a costimulatory role in the activation of CD25⁻ mediated suppressor function. The murine GITR is a 228-amino acid type I transmembrane protein with three cysteine pseudorepeats in the extracellular domain and resembles TNFRSF members CD27 and 4-1BB in the intracellular domain. Importantly, 4-1BB and CD27 molecules provide strong costimulatory signals for T cell proliferation when ligated with their respective ligands or agonistic antibodies. Four different splicing products of murine GITR have been identified and one of the variants, GITR-B, bears a unique cytoplasmic domain due to a reading frame shift. A region of the GITR-B cytoplasmic domain has significant homology with the cytoplasmic region of CD4 and CD8 that interacts with p56lck. Thus, interaction of the GITR with the GITR-L may be a costimulatory signal required for activation of the CD25⁺ T cells to exert their suppressive effects and this interaction would be blocked by the anti-GITR. The abrogation of the suppressive effects produced by activated CD25⁺ T cells may also be mediated by blocking GITR/GITR-L interactions that are maintained in the absence of restimulation of the CD25⁺ T cells via the TCR.

Although a ligand for the human GITR is known, the murine GITR-L has not been cloned. The human GITR-L was not expressed by unstimulated or stimulated T or B cells and could only be identified in umbilical vein endothelial cells. It has therefore been postulated that GITR/GITR-L interactions are important in the interaction of lymphocytes with the vascular endothelium. However, it is still possible that the murine GITR-L is expressed on other cell types including subpopulations of antigen presenting cells that are required for activation of CD25⁺ T cells. Alternatively, as multiple ligands have been identified for some of the other members of the TNFRSF, it remains possible that an unknown member of the TNFSF may function as a second GITR-L and play a role in activation of the CD25⁺ T cells. Such a ligand may function as a modulator of GITR.

CD4⁺CD25⁺ T cells have recently been identified in human thymus and peripheral blood and appear to closely resemble their murine counterparts in all their functional properties in vitro.

In order to therapeutically manipulate regulatory T cell function, one possibility is to isolate CD4⁺CD25⁺ T cells, expand them in vitro and re-infuse them into a patient with autoimmune disease or to an allograft recipient. However, this method is cumbersome as it requires large number of CD25⁺ T cells by standard procedures and individualized cellular therapies may also be impractical.

The compositions and methods presented here strongly support the view that furthering our understanding of the normal cellular physiology of regulatory T cells yields important insights into how to control both numbers and functional activity of CD25⁺ T cells in vivo. For example, inhibition of the function of the SOCS family members expressed by the CD25⁺ T cell population may be used to successfully expand the numbers of CD25⁺ cells.

One model proposed herein for the role of the GITR in costimulation of CD25 function suggests that enhancement of CD25 mediated suppression in autoimmunity might be achieved by stimulation with GITR-L, while inhibition of suppressor function by blocking GITR/GITR-L interactions with antibodies or fusion proteins might be a useful adjunct to the use of tumor vaccines or less potent vaccines to infectious agents.

What is claimed:

1. A method of potentiating immune response in a cell comprising modulating GITR with an agent that binds GITR.

2. The method of claim 1, wherein the agent that modulates GITR is an antibody.

3. The method of claim 1, wherein the agent that modulates GITR is a small molecule.

4. A method of enhancing immune response by binding GITR with an agonist to block regulatory T cell function.

5. A method of suppressing immune response by binding antagonist antibodies to GITR to potentiate regulatory T cell function.

6. A method of suppressing immune response by binding antibodies that block a GITR ligand from binding GITR to potentiate regulatory T cells.

7. A method of suppressing immune response by binding a fusion protein to a GITR ligand to modulate regulatory T cell function.

8. The method of claim 7, wherein the fusion protein that binds to a GITR ligand is GITR.Fc.

9. A method of treating a subject diagnosed with a cancer or proliferative disorder comprising administering a composition comprising an agonist of GITR and a pharmaceutically acceptable carrier.

10. A method of treating a subject diagnosed with a cancer or proliferative disorder comprising administering a composition comprising a modulator of GITR expression and a pharmaceutically acceptable carrier.

11. A method of treating a subject diagnosed with an autoimmue disorder comprising enhancing regulatory T cell function by providing a patient with an agent, inhibiting GITR function.

12. The method of claim 11, wherein the autoimmune disorder is selected from the group of consisting of Multiple Sclerosis, Insulin-Dependent Diabetes Mellitus (Type I Diabetes), Inflammatory Bowel Disease Including Ulcerative Colitis, Crohns Disease (Regional Enteritis), Systemic Lupus Erythematosis, Vasculitis, Giant cell Arteritis, Polyarteritis Nodosa, Kawasaki's Disease, Allergic Granulomatosis, Agiitis, Psoriasis, Pemphigus Vulgaris, Pemphigus Foliaceus, Bullous Pemphigoid, Cicatricial Penphigoid, Dermatitis Herpetiformis, Acute Inflammatory Demylinating Polyradiculoneuropathy (Guillain-Barre Syndrome), Chronic Inflammatory Demyleinating Polyradiculoneuropathy, Peripheral Nerve Vasculitis, Lambert-Eaton Myasthenic Syndrome, Transverse Myelitis, Optic Neuritis, Neuromyelitis Optica, Autoimmune Gastritis, Hypophysitis, Polyglandular Autoimmune Endocrine Disease, Autoimmune Thyroiditis (Graves Disease, Hashimotos Thyroiditis), Autoimmune Disease of the Adrenal, Hypoparathyroidism, Insulin Autoimmune Syndrome, Autoimmune Uveitis, Episcleritis, Scleritis, Sjorgrens Syndrome, Behcets Syndrome, Retinal Vasculitis, Myasthenia Gravis, Idiopathic Inflammatory Myopathy, Polymyositis, Dermatomyositis, Autoimmune Myocardits, Dilated Cardiomyopathy, Autoimmune Diseases of the Reproductive Glands including Oophoritis Orchitis, Premature Ovarian Failure, Aplastic Anemia, Myelodysplastic Syndromes, Paroxysmal Nocturnal Hemoglobinuria, Red Cell Aplasia, Chronic Neutropenia, Autoimmune Thrombocytopenia, Autoimmune Hemolytic Anemia, Antiphospholipid Antibody Syndromes, Pernicious Anemia, Spontaneous Acquired Inhibitors of Coagulant Factors, Autoimmune Hepatitis, Primary Biliary Cirrhosis, Hepatitis C Associated Autoimmunity, Wegeners Granulomatosis, Sarcoidosis, Scleroderma, Asthma, Allergic Rhinitis, Metal Allergy, Contact Hypersensitivity, Drug Induced Autoimmunity, Immunoglobulin a Nephropathy, Membranous Nephropathy, Idiopathic Nephritic Syndrome, Mesangiocapillary Glomerulonephritis, Poststreptococcal Glomerulonephritis, Tubulointerstitial Nephritis, Goodpastures Syndrome, and Interstitial Cystitis.

13. The method of claim 11, wherein the autoimmune disorder is selected from the group consisting of rheumatoid arthritis; systemic lupus erythematosis; psoriasis; multiple sclerosis; insulin-dependent diabetes mellitus (type I diabetes); inflammatory bowel disease including ulcerative colitis, Crohn's disease (regional enteritis); asthma; and allergic rhinitis.

14. The method of claim 11, wherein the subject requires post transplantation immune suppression.

15. A method of screening for a test compound capable of interfering with the binding of a protein encoded from a CD25⁺ differential marker (listed in Table I, or a homolog thereof), and a specific factor which binds to the protein, the method comprising:
a) combining the protein, a test compound and the specific factor which binds to the protein; and
b) determining the binding of the protein and the specific factor; and
c) correlating the amount of binding with the ability of the test compound to interfere with binding, wherein a decrease in binding of the protein and the specific factor in the presence of the test compound as compared to the absence of the test compound indicates that the test compound is capable of inhibiting binding.

16. The method of claim 15, wherein the specific factor is selected from the group consisting of a substrate for the protein, a ligand for the protein, a polynucleotide and a surface receptor.

17. The method of claim 15, wherein the test compound is selected from the group consisting of naturally-occurring compounds, biomolecules, proteins, peptides, oligopeptides, polysaccharides, nucleotides, polynucleotides, and small molecules.

18. The method of claim 15, wherein the CD25⁺ differential marker is a Cluster Type A, Cluster Type B, Cluster Type C, or Cluster Type D marker (listed in Table I, or a homolog thereof).

19. A method of screening test compounds for inhibitors of an autoimmune disorder or transplant rejection, the method comprising the steps of:
a) obtaining a sample comprising cells;
b) contacting aliquots of the sample with one of a plurality of test compounds;
c) comparing the expression levels of a CD25⁺ differential marker in each of the aliquots, wherein the CD25⁺ differential marker is selected from the group consisting of CD25⁺ differential markers (listed in Table I, or a homolog thereof); and
d) selecting one of the test compounds which substantially decreased the level of expression of a Cluster Type A or Cluster Type B CD25⁺ differential marker or which substantially increased level of expression of a Cluster Type C or Cluster Type D CD25⁺ differential marker, in the aliquot containing that test compound, relative to other test compounds.

20. The method of claim 19, wherein the test compounds are bioactive agents selected from the group consisting of proteins, oligopeptides, polysaccharides, polynucleotides, and small molecules selected from the group of libraries consisting of spatially addressable parallel solid phase or solution phase libraries or synthetic libraries made from deconvolution, ‘one-bead one-compound’ methods or by affinity chromatography selection.

21. A method of determining the severity of an autoimmune disorder or transplant rejection in a subject, the method comprising the step of comparing:
a) a level of expression of one or more CD25⁺ differential markers (listed in Table I, or a homolog thereof), in a sample from the subject; and
b) a normal level of expression of the CD25⁺ differential marker in a control sample,
wherein an abnormal level of expression of the one or more CD25⁺ differential markers in the sample from the subject relative to the normal levels is an indication that the subject is suffering from a severe autoimmune disorder or transplant rejection.

22. The method of claim 21, wherein the CD25⁺ differential marker corresponds to a transcribed polynucleotide.

23. The method of claim 21, wherein the sample is collected from blood.

24. The method of claim 21, wherein the control sample is collected from tissue substantially free of the autoimmune disorder and the abnormal increase is a factor of at least about 2.

25. The method of claim 21, wherein the level of expression of the CD25⁺ differential marker in the sample is assessed by detecting the presence in the sample of a transcribed polynucleotide or a portion thereof which hybridizes with a labeled probe under stringent conditions, wherein the transcribed polynucleotide comprises the CD25⁺ differential marker.

26. A method of modulating a level of expression of a CD25⁺ differential marker (listed in Table I, or a homolog thereof), the method comprising providing to cells of a subject an antibody which specifically binds to the CD25⁺ differential marker protein (listed in Table I, or a homolog thereof).

27. The method according to claim 26, wherein the method further comprises a therapeutic moiety conjugated to the antibody.

28. A method of localizing a therapeutic moiety to tissue having an autoimmune disorder or transplant rejection comprising exposing the tissue to an antibody which is specific to a protein encoded from a CD25⁺ differential marker which is a Surface Receptor (listed in Table I, or a homolog thereof).

29. A biochip comprising at least 5 or more CD25⁺ differential markers (listed in Table I, or a homolog thereof), wherein the biochip is utilized in high-throughput screening assays for inhibition of an autoimmune disorder or transplant rejection.

30. A composition capable of modulating an autoimmune disorder in a subject, the composition comprising one or more proteins encoded from a CD25⁺ differential marker (listed in Table I, or a homolog thereof) and a pharmaceutically acceptable carrier.

31. A composition capable of inhibiting a transplant rejection in a subject, the composition comprising one or more proteins encoded from a CD25⁺ differential marker (listed in Table I, or a homolog thereof) and a pharmaceutically acceptable carrier.

32. A therapeutic target for the inhibition of an autoimmune disorder or transplant rejection, wherein the therapeutic target comprises a CD25⁺ differential marker gene (listed in Table I, or a homolog thereof).

33. A therapeutic target for the inhibition of an autoimmune disorder or transplant rejection, wherein the therapeutic target comprises a protein encoded by a CD25⁺ differential marker (listed in Table I, or a homolog thereof).

34. A method of screening test compounds for inhibitors of a cancer or proliferative disorder in a subject, the method comprising the steps of:
a) obtaining a sample comprising cells;
b) contacting aliquots of the sample with one of a plurality of test compounds;
c) comparing the expression levels one or more CD25⁺ differential marker (listed in Table I, or a homolog thereof) in each of the aliquots; and
d) selecting one of the test compounds which substantially modulated level of expression of the CD25⁺ differential marker expression in the aliquot containing that test compound, relative to other test compounds.

35. A therapeutic target for the inhibition of a cancer or proliferative disorder, wherein the therapeutic target comprises a CD25⁺ differential marker gene (listed in Table I, or a homolog thereof).

36. A therapeutic target for the inhibition of a cancer or proliferative disorder, wherein the therapeutic target comprises a protein encoded by a CD25⁺ differential marker gene (listed in Table I, or a homolog thereof).
